(19) United States
(12) Patent Application Publication
(10) Pub. No.: US 2007/0148124 A1
(43) Pub. Date: Jun. 28, 2007

(54) Cellulose and acrylic based polymers and the use thereof for the treatment of infectious diseases

(76) Inventors: Mohamed E. Labib, Princeton, NJ (US); Robert F. Rando, Annandale, NJ (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/592,479

(22) Filed: Nov. 3, 2006

Related U.S. Application Data
(63) Continuation-in-part of application No. PCT/US05/01529, filed on Jan. 12, 2005.
Application No. PCT/US05/01529 is a continuation-in-part of application No. 10/837,153, filed on May 3, 2004.

(51) Int. Cl. A61K 31/74; C08G 63/91

(52) U.S. Cl. 424/78.18; 424/78.01; 525/54.2

(57) ABSTRACT

The present invention provides methods for the treatment or prevention of a viral, bacterial, or fungal infection using an anionic cellulose- or acrylic-based polymer, a prodrug thereof, or a pharmaceutically acceptable salt of said anionic cellulose based polymer or acrylic based polymer or prodrug of either. The present invention also provides pharmaceutical compositions comprising an anionic cellulose or acrylic based polymer, a prodrug thereof, or a pharmaceutically acceptable salt of said anionic cellulose-based polymer or prodrug. The present invention further provides combination therapies for the treatment or prevention of a viral, bacterial, or fungal infection using an anionic cellulose or acrylic-based polymer, a prodrug thereof, or a pharmaceutically acceptable salt of said anionic cellulose based or acrylic based polymer or prodrug of either and one or more anti-infective agents.

### RELATED APPLICATIONS

This is a continuation-in-part application of PCT Serial No PCT/US2005/015209 filed on May 3, 2005, which is a continuation in part of copending US Patent Application having Ser. No. 10/837,153, filed on May 3, 2004.

### FIELD OF THE INVENTION

The present invention relates to the use of anionic cellulose and acrylic based polymers for the treatment of various infectious diseases, such as sexually transmitted diseases, including viral, bacterial and fingal infections.

### BACKGROUND INFORMATION

a. Topical Treatment of Sexually Transmitted Diseases

Sexually Transmitted Diseases (STDs) are communicable diseases that can be transmitted by sexual intercourse, genital contact, or other sexual conduct. Some STDs can also be transmitted because of poor hygiene. STD pathogens are organisms that can infect tissues of the anogenital tract, the oral cavity, and the nasopharyngeal cavity. Common STD pathogens include, but are not limited to, viruses, such as human immunodeficiency virus type 1 (HIV-1), human immunodeficiency virus type 2 (HIV-2), human papillomavirus (HPV), and various types of herpes viruses, including herpes simplex virus type 2 (HSV2); bacteria, such as Trichomonas vaginalis, Neisseris gonorrhea Haemopholus ducreyl, and Chlamydia trachomatis; and fungi, such as Candida albicans.

STDs adversely affect the life of millions of people worldwide. Some STDs, such as HIV-1, can cause acquired immune deficiency syndrome (AIDS), which is fatal. In fact, the HIV/AIDS epidemic has caused approximately 3.1 million deaths worldwide since the late 1970s. Thus, there is an urgent need to treat and prevent STDs.

Despite the tremendous efforts made to develop effective treatment or preventive medicines for STDs, prophylactic vaccines against many STD pathogens are still lacking, and the most efficacious anti-infective agents are still too expensive to be widely used in developing countries. Therefore, in order to help prevent the spread of these diseases, other simple methods to control the sexual transmission of STDs must be investigated. This includes topical treatment of STDs.

Topical treatment of STDs involves local application of chemical barriers, such as microbicides, and/or mechanical barriers, such as condoms. A microbicide is an agent detrimental to, or destructive of, the life cycle of a microbe, and thus can prevent or reduce transmission of sexually transmitted infections when topically applied to the vagina or rectum. Formulations of spermicides shown in vitro to inactivate STD pathogens have been considered for use in this regard, but based upon clinical safety and efficacy trials undertaken to date, their utility remains in doubt.

For example, vaginal contraceptive products have been available for many years and usually contain nonoxynol-9 (“N-9”) or other detergent/surfactant as the active ingredient. However, N-9 has an inherent toxicity to the vaginal and cervical tissues. Frequent use of N-9 causes irritation and inflammation of the vagina (M. K. Stafford et al “Safety study of nonoxynol-9 as a vaginal microbicide: evidence of adverse effects”, J. AIDS Human Retrovirology, 17:327-331 (1998)). N-9 also can increase the potential of virus infection of the vagina by activating the local immune response and potentiating the transport of immune cells to the mucosal surface (Stevenson, J. “Widely used spermicide may increase, not decrease, risk of HIV transmission” JAMA 284:949, (2000)). Further, N-9 inactivates lactobacilli, which is the bacterium that maintains the acidic pH of the vagina (˜pH 3.5 to 5.0) by producing lactic acid and hydrogen peroxide. Disturbance of the vaginal microbial flora can lead to vaginal infections, which, in turn, can increase the chance of HIV/STD transmission. In addition, N-9 increases the permeability of vaginal tissue. Therefore, it is extremely important to identify and evaluate new antimicrobial agents which can be used intravaginally in effective doses or formulations without inactivating lactobacilli, causing overt vaginal irritation, or other side effects.

An ideal microbicide for use in the topical treatment should be safe, inexpensive, and efficacious against a broad-spectrum of microbes.

A set of criteria has been put forth to define an anti-viral microbicide that possesses desirable attributes to be a microbicide candidate with great market potential. Such an anti-viral microbicide should (i) be effective against infection caused by cell-free and cell-associated virus, (ii) adsorb tightly with its molecular target(s), i.e., its adsorption should not be reversed by dilution or washing, (iii) permanently “inactivate” the virus, (iv) inactivate free virus and infected cells faster than their rate of transport through the mucus layer, (v) have persistent activity for more than one episode of coitus, (vi) be safe to host cells and tissues, i.e., cause no irritation or lesions, (vii) be effective over a wide range of pHs found in the vaginal lumen before, during and post-coitus, (viii) be easy to formulate, (ix) remain stable in the formulated state, (x) not activate mucosal immunity, (xi) retard transport in mucus and the entire vaginal and rectal mucosa, and (xii) be inexpensive for worldwide application. It is unlikely that one candidate microbicide can fulfill all of these criteria, but these criteria nevertheless demonstrate the difficulties one may encounter in the discovery and development of an effective anti-STD agent.

Many of the compounds that are currently under evaluation or have been previously evaluated as HIV-1 microbicide candidates fall into two categories—either surfactants or polyanionic polymers (Pauwels, R., and De Clercq, E. “Development of vaginal microbicides for the prevention of heterosexual transmission of HIV”, J. AIDS Hum Retroviruses 11:211-221 (1996); “Recommendations for the development of vaginal microbicides”, International Working Group on Vaginal Microbicides AIDS 10:1-6 (1996)). Although they may satisfy some of the proposed criteria, these compounds still substantially lack desirable attributes for being an ideal microbicide according to the criteria as mentioned above. In addition, most of the microbicides under current investigation emerge from either pharmaceutical excipients or known compounds in conventional topical formulations. In fact, many of them are based on natural or synthetic water-soluble polymers that have no definite chemical formulae. Thus, these compounds are relatively non-specific compared to small molecule-based drugs. In order to satisfy the diverse criteria mentioned above, the target molecule should be custom-tailored to provide several functions at the same time. Unfortunately, the ability to manipulate, by synthetic means, the molecular structure of the current classes of agents (e.g. surfactants such as N-9 and C31G, sulfated polysaccharides, and other natural or synthetic water-soluble polymers) is limited, or in some cases even impossible. Thus, further development of these compounds as microbicides is very difficult.

For example, despite the effectiveness of inactivating HIV-1 in vitro, N-9 does not show sufficient efficacy against HIV-1 in vivo. The failure of N-9 to effectively prevent HIV-1 infection in vivo has been attributed to its high irritation profile and indiscriminate disruption of epithelial cells (Feldblum, P. J., and Rosenberg, M. J., “Spermicides and sexually transmitted diseases: new perspectives.” N.C. Med J. 47:569-572 (1986); Alexander, N. J., “Sexual transmission of human immunodeficiency virus: virus entry into the male and female genital tract”, WHO Global Programme on AIDS Fertil Steril. 54:1-18 (1990); Niruthisard, S., Roddy, R. E., and Chutivongse, S, “The effects of frequent nonoxynol-9 use on the vaginal and cervical mucosa.” Sex Transm Dis 18:176-179 (1991); Roddy, R. E., et al. “A dosing study of nonoxynol-9 and genital irritation.”, J STD AIDS 4:165-170 (1993); Kreiss et al. “Efficacy of nonoxynol 9 contraceptive sponge use in preventing heterosexual acquisition of HIV in Nairobi prostitutes.” JAMA 268:477-482 (1992); Catalone, B. J., et al. “Mouse model of cervicovaginal toxicity and inflammation for the preclinical evaluation of topical vaginal microbicides.” Antimicrobial Agents and Chemotherapy in press (2004)).

b. Sexually Transmitted Viral Infections

Despite almost 20 years of AIDS prevention efforts and research, the sexually transmitted HIV-1 and HIV-2 epidemic continues to be a major health problem throughout the world and is accelerating in many areas. At the end of 2002, the HIV epidemic had infected over 42 million people, predominantly through sexual intercourse. Of these, there have been 3.1 million cumulative deaths from the disease worldwide (statistics obtained from the Joint United Nations Program on HIV/AIDS and the World Health Organization's AIDS Epidemic Update Report, December 2002).

HIV-1 and HIV-2 are retroviruses and share about 50% homology at the nucleotide level. They contain the same complement of genes, and appear to have similar infectious cycles within human cells. The genetic material for retroviruses is Ribonucleic Acid (RNA), and encoded within their genomes are their polymerases (reverse transcriptase (“RT”), proteases and integrase enzymes essential for the viral life cycle. The RT enzyme catalyzes the synthesis of a complementary DNA strand from the viral RNA templates; the DNA helix thus formed then is inserted into the host genome with the aid of the HIV integrase enzyme. The integrated DNA may persist as a latent infection characterized by little or no production of virus or helper/inducer cell death for an indefinite period of time. When the viral DNA is transcribed and translated by the infected cells, new viral RNA and proteins are produced. The viral proteins are processed into mature entities by the viral protease enzyme, and these processed proteins are assembled into the structure of the mature virus particle.

Since the first positive identification of HIV as the causative agent in the development of AIDS, tremendous efforts have been made to develop an effective HIV vaccine. Despite the remarkable advances in the fields of molecular virology, pathogenesis and epidemiology of HIV, an effective HIV vaccine remains to be an elusive goal. The major reasons for the lack of success in the development of a vaccine include integration of the virus into the host cell genome, infections of long-lived immune cells, HIV genetic diversity (especially in its envelope), persistent high viral replication releasing up to 10 billion viral particles per day and /or production of immunosuppressive products or proteins.

Notwithstanding the technical hurdles, a variety of methods and strategies are currently being investigated in this area. For example, live attenuated simian immunodeficiency virus (SIV) has been shown to protect macaques (Daniel, M. et al. “Protective effects of a live attenuated SIV vaccine with a deletion in the nef.” Science 258:1938-1941 (1992)); however, the use of a live attenuate HIV vaccine is unlikely due to safety concerns (Baba, T., et al., “Live attenuated, multiply defected simian immunodeficiency viruses causes AIDS in infant and adult macaques.” Nature Med. 5:194-203 (1999)). Further, a number of recombinant viral vectors, such as modified vaccinia virus Ankara, canarypox virus , measles virus, and adenovirus have been evaluated in preclinical or clinical trials (Mascola, J. R., and G. J. Nabel, “Vaccines for he prevention of HIV-1 disease.” Curr. Opin. Immunol. 13:489-495 (2001); Lorin, C., et al. “A single injection of recombinant measles virus vaccines expressing human immunodeficiency virus (HIV) type 1 Clade B envelope glycoproteins induces neutralizing antibodies and cellular immune responses to HIV.” J. Virol. 78:146-157 (2004)). However, to date, these do not appear promising. Despite all of this research, at the present time and in the foreseeable future, there is no effective vaccine for HIV (either prophylactic or therapeutic).

Nevertheless, certain limited success has been achieved in the development of therapies and therapeutic regimens for the systemic treatment of HIV infections. Most compounds that are currently used or are the subject of advanced clinical trials for the treatment of HIV belong to one of the following classes:
1) Nucleoside analogue inhibitors of reverse transcriptase functions.
2) Non-nucleoside analogue inhibitors of reverse transcriptase functions
3) HIV-1 Protease inhibitors.
4) Virus fusion inhibitors (the 36 amino acid fusion inhibitor T20 has recently been approved for sale by the FDA).

Combination therapies comprising at least three anti-HIV drugs are presently the standard treatment for HIV infected patients. However, one disadvantage of the combination therapy, a.k.a. “cocktail treatment”, is the high cost associated with using multiple drugs in combination. The estimated cost for a standard combination therapy per year per person is approximately $15,000 to $20,000. This cost makes it virtually impossible for many people to afford combination therapy, especially in developing nations where the need is the greatest. Another disadvantage of the existing therapeutic regimens is the emergence of HIV mutants that are resistant to single or even multiple medications. Such drug-resistance HIV works against the population in two ways. First, the infected individual will eventually run out of treatment options; and second, if the infected individual passes along a virus already resistant to many existing therapeutic agents, the newly infected individual will have a more limited treatment option.

The HIV-1 replication cycle can be interrupted at many different points. As indicated by the approved medications, viral reverse transcriptase and protease enzymes are good molecular targets, as is the entire process by which the virus fuses to and injects itself into host cells. Thus the recently approved drug T20 (Fuzeon) is the first in a novel class of anti-HIV-1 agents. However, in addition to the drugs already approved for treatment of HIV-1 infection, work continues on the discovery and development of additional treatment modalities. This is necessary because of the propensity of the virus to mutate and thus render ineffective the existing therapies.

The search for chemotherapeutic interventions that work by novel mechanism(s) of action is particularly important in the search for new medications to combat the spread of the HIV. Several potential areas for intervention that are under consideration or have active programs include 1) blocking the viral envelope glycoprotein gp120, 2) additional mechanisms beyond gp120 to block virus entry, such as blocking the virus receptor CD4 or co-receptors CXCR4 or CCR5, 3) viral assembly and disassembly through targeting the zinc finder domain of the viral nucleocapsid protein 7 (NCp7) and 4) interfering with the functions of the viral integrase protein and interrupting virus specific transcription processes.

The mechanism by which HIV passes through the mucosal epithelium to infect underlying target cells, in the form of free virus or virus-infected cells, has not been fully defined. In addition, the type of cells infected by the virus could be derived from any one, or more, of a multitude of cell types (Miller, C. J. et al. “Genital Mucosal Transmission of Simian Immunodeficiency Virus: Animal Model for Heterosexual Transmission of Human Immunodeficiency Virus.” J. Virol. 63:4277-4284 (1989); Phillips, D. M. and Bourinbaiar, A. S. “Mechanism of HIV Spread from Lymphocytes to Epithelia.” Virology 186, 261-273 (1992); Philips, D. M., Tan X., Pearce-Pratt, R. and Zacharopoulos, V. R., “An Assay for HIV Infection of Cultured Human Cervix-derived Cells.” J. Virol. Methods, 52, 1-13 (1995); Ho, J. L. et al, “Neutrophils from Human Immunodeficiency virus (HIV)-seronegative Donors Induce HIV Replication from HIV-infected patients Mononuclear Cells and Cell lines. An In Vitro Model of HIV Transmission Facilitated by Chlamydia Trachomatis.” J. Exp. Med., 181, 1493-1505 (1995); Braathen, L. R., and Mork, C., in “HIV infection of Skin Langerhans Cells”, In: Skin Langerhans (dendritic) cells in virus infections and AIDS (ed Becker, Y.) 131-139, Kluwer Academic Publishers, Boston, (1991)). Such cells include T lymphocytes, monocytes/macrophages and dendritic cells, suggesting that CD4 cell receptors are engaged in the process of virus transmission as is well documented for HIV infection in blood or lymphatic tissues (Parr M. B., and Parr E. L., “Langerhans Cells and T lymphocytes Subsets in the Murine Vagina and Cervix.” Biology and Reproduction 44,491-498 (1991); Pope, M. et al. “Conjugates of Dendritic Cells and Memory T Lymphocytes from Skin Facilitate Productive Infection With HIV-1.” Cell 78, 389-398 (1994); and Wira, C. R. and Rossoll, R. M. “Antigen-presenting Cells in the Female Reproductive Tract: Influence of Sex Hormones on Antigen Presentation in the Vagina.” Immunology, 84, 505-508 (1995)).

Therefore, the need for efficacious, safe, and inexpensive anti-viral agents to treat or prevent the transmission of HIV (in lieu of a vaccine) is evident.

Besides HIV, herpes viruses also infect humans (“Herpesviridae; A Brief Introduction”, Virology, Second Edition, edited by B. N. Fields, Chapter 64, 1787 (1990)) and cause STDs. Some common herpes viruses are described below. However, the list is not meant to be exhaustive, but only illustrative of the problem.

Herpes Simplex Virus Type 1 (HSV1) is a recurrent viral infection characterized by the appearance on the cutaneous or mucosal surface membranes of single or multiple clusters of small vesicles filled with clear fluid on a slightly raised inflamed base (herpes labialis). In addition, gingivostomatitis may occur as a result of HSV1 infection in infants (Kleymann, G., “New antiviral drugs that target herpes virus helicase primase enzyme.” Herpes 10:46-52 (2003); “Herpesviridae; A Brief Introduction”, Virology, Second Edition, edited by B. N. Fields, Chapter 64, 1787 (1990)).

Herpes Simplex Virus Type 2 (HSV2) causes genital herpes, and vulvovaginitis may occur as a result of HSV2 infection in infants (Kleymann, G., “New antiviral drugs that target herpes virus helicase primase enzyme.” Herpes 10:46-52 (2003)).

Human Cytomegalovirus (HCMV) infections are a common cause of morbidity and mortality in solid organ and haematopoietic stem cell transplant recipients (Razonable, R. R., and Paya, C. V., “Herpes virus infections in transplant recipients: current challenges in the clinical management of cytomegalovirus and Epstein-Barr virus infections.” Herpes 10:60-65 (2003)).

Varicella-Zoster Virus (VZV) causes varicella (chickenpox) and Zoster (shingles) (Vazquez, M., “Varicella Zoster virus infections in children after introduction of live attenuated varicella vaccine.” Curr. Opin. Pediatr. 16:80-84 (2004)).

Epstein-Barr virus (EBV) is the causative agent of infectious mononucleosis and has been associated with Burkett's lymphoma and nasopharyngeal carcinoma. Human Herpes virus 6 (HHV6) is a very common childhood disease causing exanthem subitum (roseola) (Boutolleau, D., et al., “Human herpes virus (HHV)-6 and HHV-7; two closely related viruses with different infection profiles in stem cell transplant recipients”, J. Inf. Dis. (2003)).

Herpes Simplex Virus Type 7 (HSV7) is a T-lymphotropic herpes virus and can cause exanthem subitum. The pathogenesis and sequelae of HH7, however, are poorly understood (Dewhurst, S., Skrincosky, D., and van Loon, N. “Human Herpes virus 7”, Expert Rev Mol. Med. 18:1-10 (1997)).

Herpes Simplex Virus Type 8 (HSV8) is another herpes virus. The molecular genetics of the human herpes virus 8 (HHV8) has now been characterized, and the virus appears to be important in the pathogenesis of Kaposi's sarcoma (KS) (Hong, a, Davies, S. and Lee, S. C., “Immunohistochemical detection of the human herpes virus 8 (HHV8) latent nuclear antigen-1 in Kaposi's sarcoma.” Pathology 35:448-450 (2003); Cathomas, G., “Kaposi's sarcoma-associated herpes virus (KSHV)/human herpes virus 8 (HHV8) as a tumor virus.” Herpes 10:72-77 (2003)).

In addition to infections in humans, herpes viruses have also been isolated from a variety of animals and fish (“Herpesviridae; A Brief Introduction.” Virology, Second Edition, edited by B. N. Fields, Chapter 64, 1787 (1990)).

Herpes viruses are large double stranded DNA viruses, with genome sizes usually greater than 120,000 base pairs (for review see “Herpesviridae; A Brief Introduction”, Virology, Second Edition, edited by B. N. Fields, Chapter 64, 1787 (1990)). HSV1 is one of the most common infections in the U.S. with infection rates estimated to be greater than 50% of the population. All herpes virus types encode their own polymerase, and many times, their own thymidine kinase. For this reason, most of the antiviral agents target the DNA polymerase enzyme of the virus and/or use the viral thymidine kinase for conversion from prodrug to active agent, thereby gaining specificity for the infected cell.

Unfortunately, the herpes viruses are another class of viruses that, like HIV-1, develop resistance to existing therapy, and can cause problems from a STD as well as a chronic infection point of view. For example, human cytomegalovirus (HCMV) is a serious, life threatening opportunistic pathogen in immuno-compromised individuals such as AIDS patients (Macher, A. M., et al., “Death in the AIDS patients: role of cytomegalovirus.” NEJM 309:1454 (1983); Tyms, A. S., Taylor, D. L., and Parkin, J. M., “Cytomegalovirus and the acquired immune deficiency syndrome.” J Anitmicrob Chemother 23 Supplement A: 89-105 (1989)) and organ transplant recipients (Meyers, J. D., “Prevention and treatment of cytomegalovirus infections.” Annual Rev. Med. 42:179-187 (1991)). Over the past decade, there has been a tremendous effort dedicated to improving the available treatments for herpes viruses. At the present time, acyclovir is still the most prescribed drug for HSV1 and HSV2, while ganciclovir, foscarnet, cidofovir, and fomivirsen are the only drugs currently available for HCMV (Bédard et al., “Antiviral properties of a series of 1,6-naphthyridine and dihydroisoquinoline derivatives exhibiting potent activity against human cytomegalovirus.” Antimicrob. Agents and Chemother. 44:929-937 (2000)). However, none of these systemic treatments are effective in preventing the sexual transmission of viruses; therefore, there is still an urgent need for new drugs that have unique mechanisms of action and modes of therapeutic intervention.

While HSV1 infections are more common than HSV2, it is still estimated that up to 20% of the U.S. population are infected with HSV2. HSV2 is associated with the anogenital tract, is sexually transmitted, causes recurrent genital ulcers, and can be extremely dangerous to newborns (causing viremia or even a fatal encephalitis) if transmitted during the birthing process (Fleming, D. T., McQuillan, G. M. Johnson, R. E. et al. “Herpes simplex virus type 2 in the United States, 1976 to 1994.” N. Eng. J. Med 337:1105-1111 (1997); Arvin, A. M., and Prober, C. G., “Herpes Simplex Virus Type 2—A Persistent Problem.” N. Engl. J. Med. 337:1158-1159 (1997)). Although, as stated above, there are treatments available for HSV1 and HSV2, efficacious long-term suppression of genital herpes is expensive (Engel, J. P. “Long-term Suppression of Genital Herpes.” JAMA, 280:928-929 (1998)). The probability of further spread of the virus by untreated people and asymptomatic carriers not receiving antiviral therapy is extremely high, considering the high prevalence of the infections. It is thought that other herpes viruses, including HCMV (Krieger, J. M., Coombs, R. W., Collier, A. C. et al. “Seminal Shedding of Human Immunodeficiency virus Type 1 and Human Cytomegalovirus: Evidence for Different Immunologic Controls.” J. Infect. Dis. 171:1018-1022 (1995); van der Meer, J. T. M., Drew, W. L., Bowden, R. A. et al. “Summary of the International Consensus Symposium on Advances in the Diagnosis, Treatment and Prophylaxis of Cytomegalovirus Infection.” Antiviral Res. 32:119-140 (1996)), herpes virus type 6 (Leach, C. T., Newton, E. R., McParlin, S. et al. “Human Herpes virus 6 Infection of the female genital tract.” J. Infect. Dis. 169:1281-1283 (1994)), and herpes virus type 8 (Howard, M. R., Whitby, D., Bahadur, G. et al. “Detection of Human Herpes virus 8 DNA in Semen from HIV-infected Individuals but Not Healthy Semen Donors.” AIDS 11:F15-F19 (1997)) are also transmitted sexually.

Vaccines for herpes viruses are extremely limited. A vaccine based on the OKA strain of varicella zoster virus is commercially available, but, to date, no therapeutic or prophylactic herpes vaccinations that can treat or stop the spread of other herpes diseases are available (Kleymann, G., “New antiviral drugs that target herpes virus helicase primase enzymes.” Herpes 10:46-52 (2003)). At the present time, there are several ongoing efforts to develop effective vaccines against HSV1 and HSV2, most of which focus on key glycoproteins on the viral envelope (Jones, C. A., and Cunningham, A. L., “Development of prophylactic vaccines for genital and neonatal herpes.” Expert Rev. Vaccines 2:541-549 (2003); Cui, F. D., et al., “Intravascular naked DNA vaccine encoding glycoprotein B induces protective humoral and cellular immunity against herpes simplex virus type 1 infection in mice.” Gene Therapy 10:2059-2066 (2003)).

Therefore, at the present time, there is an urgent need for efficacious, safe, and inexpensive antiviral agents that can treat or prevent the transmissions of various herpes viruses.

c. Sexually Transmitted Bacterial Infections.

Sexually transmitted infections of bacterial origin are among the most common infectious diseases in the United States and throughout the world. In the U.S. alone, there were conservative estimates of over 4 million new cases in 1996 of three major bacterial infections, namely syphilis, gonorrhea (Neisseria gonorrhea), and Chlamydia (U.S. Government, National Institutes of Health, National Institutes of Allergy and Infectious Disease web site (factsheets/stdinfo)). Even this large number of infections is under-estimating the true prevalence of these diseases. The dramatic under-reporting of STDs is due to the reluctance of infected individuals to discuss their sexual health issues. In fact, it has been estimated that in addition to the approximate 600,000 cases of Chlamydia reported in 1999, an additional 3 million unreported cases occurred (U.S. Government, Center for Disease Control and Prevention, National Center for HIV, STD, and TB Prevention, Division of Sexually Transmitted Diseases web site (nchstp/dstd)). In addition, worldwide, there is over a 300 million annual incidence of bacterial STDs (Gerbase, A. C., Rowley, J. T., Heymann, D. H. L., et al. “Global prevalence and incidence estimates of selected curable STDs.” Sex. Transm. Inf. 74 (suppl. 1): S12-S16 (1998)).

Although many types of bacterial infections can be treated with antibiotics that are relatively inexpensive compared to the antiviral agents, the effectiveness of these antibiotics in treating bacterial infections continues to deteriorate because of the ever-growing antibiotic-resistance problem. In fact, even the once easily curable gonorrhea has become resistant to many of the traditional antibiotics. For this reason alone, new and efficacious anti-bacterial agents that can treat or prevent the sexually transmitted bacterial infections are urgently needed.

d. Influenza

Another major viral infection afflicting a large proportion of the population is the influenza virus. Influenza continues to be a serious health concern causing substantial morbidity and mortality, particularly among the very young, the elderly, and people with chronic cardiovascular and respiratory diseases. Vaccine development is only partially effective in the control of influenza epidemics due, at least in part, to the rapid change in the antigenic sites of the surface proteins of the influenza virus. In addition, there is concern that it will not be possible to generate and manufacture new vaccines rapidly enough to protect against future pandemic influenza virus strains, which arise due to major changes in the antigenic determiinants. Thus, effective antiviral agents would provide an attractive therapeutic option, particularly in the event of the occurrence of a pandemic strain.

Two classes of anti-influenza virus antiviral agents which target either the M2 ion channel or the neuraminidase enzyme are currently available for influenza management and are under consideration for stockpiling in the event of an influenza pandemic. However, use of the M2 blockers, amantadine and rimantadine is limited by a lack of inhibitory effect against influenza B viruses, side effects, and a rapid emergence of antiviral resistance. M2 inhibitor-resistant variants are transmissible from person to person, are pathogenic, and can be recovered occasionally from untreated individuals. Importantly, recent human isolates of highly virulent A/H5N1 influenza viruses are naturally resistant to these drugs.

Along with the M2 inhibitors, the two neuraminidase inhibitors (NAIs), oseltamivir and zanamivir, are the only antiviral agents approved for the prophylaxis and/or treatment of influenza virus infections. The influenza virus, neuraminidase, is an attractive antiviral target because the enzyme active site is highly conserved among all influenza A and B virus strains investigated, and the enzymatic mechanism of action has been studied down to the atomic level, facilitating the possibility of rationally based drug design. The recent commercialization of oseltamivir and zanamivir has demonstrated that the influenza virus neuraminidase enzyme is a valid target for antiviral intervention. It is interesting to note that while the efficacy of zanamivir is well documented, including in humans, due to poor oral bioavailability and rapid renal elimination, zanamivir is applied to the respiratory tract via an intranasal spray or by inhalation.

All of these approved compounds have limitations, such as significant adverse side effects and the rapid emergence of resistant strains in the clinical setting. In fact, as mentioned above, treatment with M2 ion channel blockers can cause emergence of fully pathogenic and transmissible resistant variants in at least 30% of individuals. As a result, there has been a great deal of interest in identifying novel antiviral agents directed against influenza viruses.

e. Cellulose or Acrylic based Polymers as Antimicrobial Agents

Recent work conducted at the New York Blood Center has focused on the use of two promising anionic polymers, cellulose acetate phthalate (CAP) and hydroxypropyl methylcellulose phthalate (HPMCP). Both of these polymers have demonstrated excellent activity against a wide range of sexually transmitted organisms, including HIV-1 (U.S. Pat. Nos. 6,165,493; 6,462,030; Neurath, A. R., et al. “Anti-HIV-1 activity of cellulose acetate phthalate: Synergy with soluble CD4 and induction of “dead-end” gp41 six-helix bundles.” BMC Infectious Diseases 2:6 (2002); Neurath, A. R., Strick, N., Li, Y. Y., and Jiang, S., “Design of a “microbicide” for prevention of sexually transmitted diseases using “inactive” pharmaceutical excipients.” Biologicals 27:11-21 (1999); Gyotoku, T., Aurelian, L., and Neurath, A. R. “Cellulose acetate phthalate (CAP): an ‘inactive’ pharmaceutical excipient with antiviral activity in the mouse model of genital herpes virus infection.” Antiviral Chem. Chemother 10:327-332 (1999); Neurath, A. R., Li, Y. Y., Mandeville, R., and Richard, L., “In vitro activity of a cellulose acetate phthalate topical cream against organisms associated with bacterial vaginosis.” J. Antimicrobial Chemother. 45:713-714 (2000); Neurath, A. R. “Microbicide for prevention of sexually transmitted diseases using pharmaceutical excipients.” AIDS Patient Care STDS 14:215-219 (2000); Manson, K. H. Wyand, M. S., Miller, C., and Neurath, A. R. “The effect of a cellulose acetate phthalate topical cream on vaginal transmission of simian immunodeficiency virus in rhesus monkeys.” Antimicrob. Agents Chemother 44:3199-3202 (2000); Neurath, A. R., Strick, N., Li, Y. Y., and Debnath, A. K. “Cellulose acetate phthalate, a common pharmaceutical excipient, inactivates HIV-1 and blocks the coreceptor binding site on the virus envelope glycoprotein gp120.” BMC Infectious Diseases 1:17 (2001)).

CAP and HPMCP were first developed for use as pharmaceutical excipients in enteric coating to protect pharmaceutical preparations from degradation by the low pH of gastric juices and to simultaneously protect the gastric mucosa from irritation by the drug. One desirable attribute of these coatings was the low solubility in gastric juices. That is, CAP and HPMCP slightly dissolve until they reach the intestines where the pH is mostly neutral or alkaline. There is a large difference in pH between the stomach and the intestines. In the stomach gastric juice, pH values range from 1.5 to 3.5 while in the intestines, the pH values are much higher, ranging from 3.6 to 7.9. The pH in the duodenum closest to the stomach has a lower pH due to the transfer of material from the stomach to the intestines; however, at the point of nutrient uptake by the intestines, the pH has moved into the neutral or slightly alkaline range (“Remington's Pharmaceutical Sciences,” 14^{th} ed., Mack Publishing Co., Easton, Penn., 1970, p. 1689-1691; Wagner, J. G., Ryan, G. W., Kubiak, E., and Long, S., “Enteric Coatings V. pH Dependence and Stability”, J. Am. Pharm. Assoc. Sci., 49:133-139, (1960); Kokubo, H., et al., “Development of Cellulose derivatives as novel enteric coating agents soluble at pH 3.5 -4.5 and higher”, Chem. Pharm. Bull 45:1350-1353 (1997)). Commercially available enteric coating agents of both cellulosic and acrylic polymers are soluble in the pH ranging from 5.0 to 7.0 (Kokubo, H., et al., “Development of Cellulose derivatives as novel enteric coating agents soluble at pH 3.5 -4.5 and higher.” Chem. Pharm. Bull 45:1350-1353 (1997); Maekawa, H., Takagishi, Y., Iwamoto, K., Doi, Y., and Ogura, T. “Cephalexin preparation with prolonged activity.” Jpn J. Antibiot. 30:631-638 (1977); Lappas, L. C., and McKeeham, W., “Polymeric pharmaceutical coating materials. II. In vivo evaluation as enteric coatings.” J. Pharm. Sci., 56:1257-261 (1967); Hoshi, N., Kokubo, H., Nagai, T., Obara, S. “Application of HPMC and HPMCAS to film coating of pharmaceutical dosage forms in aqueous polymeric coatings for pharmaceutical dosage forms.” 2^{nd} ed. By McGinty, J. W., Marcel Decker, Inc., New York and Basel, 1997, pp. 177-225). However, in drugs with poor and limited absorbability in the gastro-intestinal tract, it is desirable to ensure that the coating is dissolved as early as possible by reducing the dissolution pH thereof, in order to maximize the drug absorption. This problem in solubility at low pH (3.5 to 5.5) has been found to be the case for both CAP and HPMCP. CAP and HPMCP are insoluble in aqueous solutions unless the pH is ˜6.0 or above (Neurath A. R. et al. “Methods and compositions for decreasing the frequency of HIV, Herpes virus and sexually transmitted bacterial infections.” U.S. Pat. No. 6,165,493 (2000)).

This differential in pH solubility is of a great concern for agents that have potential use as inhibitors of sexually transmitted diseases. Vaginal secretions from healthy, reproductive-age women are usually acidic with pH values in the range of 3.4 to 6.0 (S. Voeller, D. J. Anderson, “Heterosexual Transmission of HIV” JAMA 267, 1917-1918 (2000)). The pH of the vaginal lumen may then fluctuate transiently upon the addition of semen. Consequently the topical application of a formulation in which either CAP or HPMCP would be soluble (i.e. pH ˜6.0) would be expected to precipitate out of solution once they come in contact with the “acidic” vaginal environment. Furthermore the dissolution rate of this class of compounds is so slow that the active agent may not have time to regain solubility post-coitus when the pH has been transiently raised (Kokubo, H., et al., “Development of Cellulose derivatives as novel enteric coating agents soluble at pH 3.5-4.5 and higher”, Chem. Pharm. Bull 45:1350-1353 (1997). Moreover, if the polyanionic electrostatic nature of the molecules is diminished due to lack of dissociation of the molecule's carboxyl group in the vagina, the protective property of the molecule is expected to decrease or even disappear completely. It is therefore of interest from both a pharmaceutical coating point of view and from a putative topical microbicide perspective that polymers soluble at more acidic pH than conventional enteric coatings are designed and tested for biological or pharmacological benefit.

As stated above, the original utility of CAP and HPMCP was with respect to enteric coating. Another class of molecules widely used in pharmaceutical applications for their excellent film-forming properties and high quality bio-adhesive performance is acrylic co-polymers that also contain a periodic carboxylic acid group. Gantrez (Gantrez® International Specialty Products or ISP) is one such co-polymer made from the polymerization of methylvinyl ether and maleic anhydride (poly methyl vinyl ether/maleic anhydride (MVE/MA)). MVE/MA and similar agents are used as thickeners, complexing agents, denture adhesive base, buccal/transmucosal tablets, transdermal patches (Degim, I. T., Acarturk, F, Erdogan, D., and Demirez-Lortlar, N. “Transdermal administration of bromocriptine.” Biol. Pharm. Bull. 26:501-505, (2003)), topical carriers or microspheres for mucosal delivery of drugs (Kockisch, S., Rees, G. D., Young, S. A., Tsibouklis, J., and Smart, J. D. “Polymeric microspheres for drug delivery to the oral cavity: an in vitro evaluation of mucoadhesive potential.” J. Pharm. Sci. 92:1614-1623, (2003); Foss, A. C., Goto, T., Morishita, M., and Peppas, N. A., “Development of acrylic based copolymers for oral insulin delivery.” Eur. J., Pharm. Biopharm. 57:163-169, (2004)), enteric film coating agents, wound dressing applications (Tanodekaew, S., Prasitsilp, M., Swasdison, S., Thavomyutikam, B., Pothsree, T., and Pateepasen, R. “Preparation of acrylic grafted chitin for wound dressing application.” Biomaterials: 1453-1460 (2004)), and hydrophilic colloids. One form of Gantrez is mixed with triclosan in toothpaste with claims of extended control of breath odor for over 12 hours (Sharma, N. C., Galustians, H. J., Qaquish, J., Galustians, A., Rustogi, K. N., Petrone, M. E., Chanknis, P. Garcia, L., Volpe, A. R., and Proskin H. M., “The clinical effectiveness of dentifrice containing triclosan and a copolymer for controlling breath odor measured organoleptically twelve hours after tooth brushing.” J. Clin. Dent. 10:1310134, (1999); Zambon, J. J., Reynolds, H. S., Dunford, R. G., and Bonta, C. Y., “Effect of triclosan/copolymer/fluoride dentifrice on the oral microflora.” Am. J. Dent. 3S27-34, (1990)). Certain acrylic based copolymers are also being studied for use in diagnosis of cancer (Manivasager, V., Heng, P. W., Hao, J., Zheng, W., Soo, K. C., and Olivo, M. “A study of 5-aminolevulinic acid and its methyl ester used in in vitro and in in vivo system so human bladder cancer.” Int. J. Oncol. 22:313-318, (2003)). Maleic acid copolymers with methyl vinyl ether are also being used in model systems to covalently immobilize peptides and other macromolecules via the formation of amide bonds (Ladaviere, C., Lorenzo, C., Elaissari, A., Mandrand, B., and Delair, T. “Electrostatically driven immobilization of peptides onto (Maleic anhydride-alt-methyl vinyl ether) copolymers in aqueous media.” Bioconj. Chem. 11:146-152, (2000)). Divinyl ether and maleic anhydride copolymers have been used to retard the development of artificially induced metastases and to activate macrophages to non-specifically attack tumor cells (Pavlidis, N. A., Schultz, R. M., Chirigos, M. A. and Luetzeler, J. “Effect of maleic anhydride-divinyl ether copolymers on experimental M109 metastases and macrophage tumoricidal finction.” Cancer Treat Rep. 62:1817-1822, (1978)). In these studies, the investigators found that the lower molecular weight polymers were most effective. This is similar to the results obtained using divinyl ether and maleic anhydride copolymers linked to derivatives of the antiviral agent, adamantine (Kozeletskaia, K. N., Stotskaia, L. L., Serbin, A. V., Munshi, K., Sominina, A. A., and Kiselev, O.I. “Structure and antiviral activity of adamantine-containing polymer preparation.” Vopr VIrousol. 48:19-26, (2003)). In experiments, the adamantine containing copolymers were shown to inhibit a variety of viruses in vitro including influenza, herpes simplex type 1, and parainfluenza. The efficiency of the antiviral effect, however, depended upon the molecular weight of the polymer (lower molecular weight was better) and the structure of the linkage between the adamantine and the copolymer. But, no one has utilized these compounds for the treatment of bacterial, viral, or fingi infections.

The present invention overcomes many of the problems described hereinabove. As shown hereinbelow, the applicants provide certain anionic cellulose and acrylic based polymers that are soluble in aqueous solution at pH from about 3 to about 14 and the use of such anionic cellulose and acrylic based polymers to treat various infectious diseases including STDs.

These anionic cellulose and acrylic based polymers of the present invention are efficacious, safe, and inexpensive.

### SUMMARY OF THE INVENTION

The present invention is directed to a method for the treatment or prevention of a viral, bacterial, or fingal infection in a host, which comprises administering to the host a therapeutically effective amount of an anionic cellulose or acrylic based polymer, a prodrug of said anionic cellulose or acrylic based polymer or a pharmaceutically acceptable salt of said anionic cellulose or acrylic based polymer or prodrugs of either.

The present invention is also directed to anionic cellulose or acrylic based polymers which are molecularly dispersed and mostly ionically dissociated in an aqueous solution at pH ranging from about 3 to about 5.

The present invention is also directed to the use of a polymer for the treatment of a viral, a bacterial, or a fingal infection comprising administering to a host a therapeutically effective amount of said polymer comprised of the following repeating unit 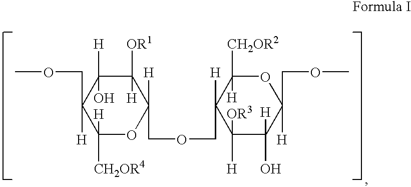
or pharmaceutically acceptable salts thereof;
wherein each R¹, R², R³, and R⁴ are the same or different, and are hydrogen, C₁-C₆ hydroxyalkyl, an aliphatic group, preferably C₁-C₆ alkyl, an alicyclic group, an aryl group, a arylaliphatic, or an heteroring group or 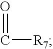
   wherein each of said aliphatic group, alicyclic group, aryl group, and heteroring group is independently unsubstituted or substituted by one or more substituents selected from the group consisting of carboxylic acid, sulfuric acid, sulfonic acid, carboxylate, sulfate, sulfonate, and acidic anhydride; R⁷ is hydrogen, C₁-C₆ hydroxyalkyl, an aliphatic group, preferably C₁-C₆ alkyl, alicyclic group, an aryl group arylaliphatic, or an heteroring group, wherein the aliphatic groups, alicyclic groups, aryl group and heteroring are independently unsubstituted or substituted by one or more substituents selected from carboxylic acid, sulfuric acid, sulfonic acid, carboxylate, sulfate, sulfonate and acidic anhydride, however, at least one of R¹, R², R³ and R⁴ contains at least one COOH group or salt thereof wherein the pKa of one of the COOH groups present or if its salt is present the pKa of the corresponding acid is less than about 5.0.

The present invention also provides polymers described hereinabove wherein said aliphatic group, alicyclic group, aryl group, or heteroring group is further substituted with one or more hydroxyl groups.

The present invention also provides polymers described hereinabove wherein said acidic anhydride is derived from acids chosen from the group consisting of acetic acid, sulfobenzoic acid, phthalic, trimellitic acid, and other carboxylic acids; and wherein said acidic anhydride can be derived from two of the same or different carboxylic acids.

The present invention also provides polymers described hereinabove wherein at least one of R¹, R², R³, and R⁴ is chosen from the group consisting of trimellitic acid, trimesic acid, hemimellitic acid, maleic acid, succinic acid, diethylmalonic acid, trans-aconitic acid, 1,8-naphthalic anhydride, 1,4,5,8-naphthalene tetracarboxylic acid dianhydride, 2-sulfobenzoic acid cyclic anhydride, 4-sulfo-1,8-naphthalic anhydride, tartaric acid, D-mallic acid, L-mallic acid, and vinyl acetic acid, and the remainder is as defined hereinabove.

In a preferred embodiment of the present invention, polymers described hereinabove include hydroxylpropyl methyl cellulose (HPMC) based polymers, cellulose acetate (CA) based polymers, hydroxylpropyl methylcellulose trimellitate (HPMCT) based polymers, hydroxylpropyl methylcellulose acetate maleate (HPMC-AM) based polymers, hydroxylpropyl methylcellulose acetate sulfobenzoate based polymers, cellulose acetate trimellitate based polymers, and cellulose acetate sulfobenzoate based polymers.

The present invention is also directed to the use of an acrylic based polymer for the treatment of a viral, a bacterial, or a fungal infection comprising administering to a host a therapeutically effective amount of said acrylic based polymer comprised of the following repeating unit 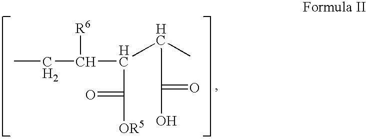
or pharmaceutically acceptable salts thereof;
wherein each R⁵ is hydrogen, an aliphatic group, an alicyclic group, an aryl group, aryl aliphatic or an heteroring group; wherein each of said aliphatic group, alicyclic group, aryl group, or heteroring group is independently unsubstituted or substituted by an aliphatic group, alicyclic group, an aryl or aryl aliphatic or aliphatic aryl group or R⁵ is 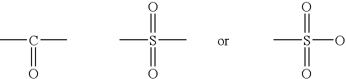
   wherein 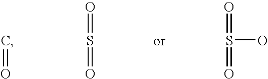
   group is bonded to an aliphatic group, aryl group, alicyclic group or heteroring, which may be unsubstituted or substituted by one or more carboboxylic acid moiety, sulfonic acid moiety, sulfuric acid moiety and optionally with hydroxy or halide and each R⁶ is hydrogen, C₁-C₆ alkyl or C₁-C₆ hydroxyalkyl, aryl or SR⁸ or OR⁸, wherein each R⁸ is hydrogen, aliphatic group, alicyclic group, aryl group, or aryl aliphatic or heteroring which R⁶ may be unsubstituted or substituted with an aliphatic group, alicyclic group or aryl group, or aryl aliphatic group.

The present invention also provides acrylic based polymers described hereinabove wherein said aliphatic group, alicyclic group, aryl group, or heteroaryl group is further substituted with one or more hydroxyl groups.

In an embodiment, the present invention provides acrylic based polymers described hereinabove wherein R⁵ is chosen from the group consisting of trimellitic acid, trimesic acid, hemimellitic acid, maleic acid, succinic acid, diethylmalonic acid, trans-aconitic acid, 1,8-naphthalic anhydride, 1,4,5,8-naphthalene tetracarboxylic acid dianhydride, 2-sulfobenzoic acid cyclic anhydride, 4-sulfo-1,8-naphthalic anhydride, tartaric acid, D-mallic acid, L-mallic acid, and vinyl acetic acid.

The present invention also provides acrylic based polymers described hereinabove wherein R⁶ is lower alkyl especially methyl.

In a preferred embodiment of the present invention, acrylic based polymers described hereinabove include methyl vinyl ether and maleic anhydride (MVE/MA)-based polymers or alternating copolymers and polystyrene maleic anhydride-based polymers or alternating copolymers.

The present invention also provides a method for the treatment or prevention of a viral, bacterial, or fungal infection in a host, which comprises administering to the host a therapeutically effective amount of an anionic cellulose-based polymer or acrylic based polymer, a prodrug of either the cellulose based polymer or acrylic based polymer, or a pharmaceutically acceptable salt of said anionic cellulose based polymer, acrylic based polymer or prodrug of either or combination thereof.

More particularly, the present invention provides such methods utilizing the cellulose-base polymer as described herein including that of Formula I or a pharmaceutically acceptable salt thereof or prodrug or the acrylic based polymer described hereinabove, inducting that of Formula II or pharmaceutically acceptable salt thereof or prodrug, as described herein, wherein the viral infection is caused by viruses including HIV-1, HIV-2, HPV, HSV1, HSV2, RSV, (respiratory syncytial virus), VZV, and influenza virus, including both type A, e.g., H5N1, and type B, rhinovirus, SARS (severe acute respiratory syndrome) causing virus, Small Pox virus, Cow pox, Vaccinia virus, heamorraghic fever causing viruses, such as the Filoviruses Marburg and Ebola, the Arena viruses such as Lassa Fever Virus and New World Arenaviridae, the Bunyaviruses such as Crimean-Congo hemorrhagic virus, Hanta viruses, Punta Toro and Rift Valley Fever Viruses, and the Flaviruses such as Hepatitis C virus, Dengue and Yellow Fever Viruses, and the like.

In an embodiment, the present invention is directed to the treatment or prophylaxis of a viral infection in a subject by administering thereto a therapeutically or prophylactically effective amount of a compound of Formula I or II or combination thereof.

In another method, the present invention is directed to the treatment or prevention of bacterial infections utilizing the cellulose-base polymer or pharmaceutically acceptable salt thereof or prodrug or the acrylic based polymer or pharmaceutically acceptable salt thereof or prodrug, as described herein, or combination thereof in effective therapeutic or prophylactic amounts, respectively. In another embodiment, the present invention is directed to the treatment of or prophylaxis of bacterial infections utilizing compounds described hereinabove, wherein the bacterial infection is caused by bacteria including Trichomonas vaginalis, Neisseris gonorrhea Haemopholus ducreyl, Chlamydia trachomatis, Gardnerella vaginalis, Mycoplasma hominis, Mycoplasma capricolum, Mobiluncus curtisii, Prevotella corporis, Calymmatobacterium granulomatis, and Treponema pallidum.

In another embodiment, the present invention provides a method for treating or preventing fungal infections in a subject by administering the cellulose base polymer or pharmaceutically acceptable salt thereof or prodrug or the acrylic based polymer or pharmaceutically acceptable salt thereof or prodrug, or combination thereof, as described herein, in therapeutically or prophylatically effective amounts. In another embodiment, the present invention is directed to treating or preventing a fungal infection in a subject by administering thereto a therapeutically or prophylatically effective amount of the cellulose based polymer or pharmaceutically acceptable salt thereof or prodrug, as described herein or the acrylic based polymer or pharmaceutically acceptable salt thereof or combination of any of the foregoing, wherein the fungal infection is caused by fungi including Candida albicans.

In a further embodiment, the present invention is directed to the treatment or prophylaxis in a subject of a disease caused by or associated with an infection by a bacteria, virus or fungus, especially the ones listed hereinabove comprising administering to said subject the acrylic based polymer or pharmaceutically acceptable salt thereof or prodrug, as described herein, or the cellulose based polymer or pharmaceutically acceptable salt thereof or prodrug or combination of any of the foregoing in therapeutically or prophylatically effective amounts, respectively.

The present invention is also directed to a pharmaceutical composition comprising a therapeutically effective amount of an anionic cellulose-based polymer or a pharmaceutically acceptable salt thereof or prodrug thereof or an anionic acrylic-based polymer or pharmaceutically acceptable salt thereof or a prodrug thereof or a combination thereof in association with a pharmaceutically acceptable carrier, vehicle, or diluent.

The present invention is also directed to polymers having repeating units of Formula I or II, as described herein or pharmaceutically acceptable salts of polymers of Formula I or II or prodrugs of polymers of Formula I or II for the utility described herein.

The present invention also provides pharmaceutical compositions comprising a therapeutically effective amount of the anionic cellulose-based polymer or the anionic acrylic-based polymer described herein, a prodrug of either said anionic cellulose-based polymer or anionic acrylic-based polymer, or a combination thereof or a pharmaceutically acceptable salt of said anionic cellulose based polymer or acrylic-based polymer or prodrug; and a pharmaceutically acceptable carrier, vehicle or diluent. The pharmaceutical compositions can be delivered in a liquid or solid dosage form. Alternatively, the pharmaceutical compositions can be incorporated into barrier devices such as condoms, diaphragms, or cervical caps. The pharmaceutical compositions described herein are useful for the treatment of a virus, bacterial, or fungal infection in a host.

The present invention also provides methods for the treatment or prevention of a virus, bacterial, or fungal infection in a host, which comprises administering to the host a therapeutically effective amount of an anionic cellulose-based polymer, a prodrug thereof, or a pharmaceutically acceptable salt of said anionic cellulose-based polymer or prodrug in combination with one or more anti-infective agents. More particularly, the one or more anti-infective agents can be an anti-viral agent, an anti-bacterial agent, an anti-fungal agent, or a combination thereof. Further, the anionic cellulose-based polymer and the one or more anti-infective agents can be administered simultaneously or sequentially.

In another embodiment, said one or more anti-infective agents in such methods include antiviral protease enzyme inhibitors (PI), virus DNA or RNA or reverse transcriptase (RT) polymerase inhibitors, virus/cell fusion inhibitors, virus integrase enzyme inhibitors, virus/cell binding inhibitors, and/or virus or cell helicase enzyme inhibitors, bacterial cell wall biosynthesis inhibitors, virus or bacterial attachment inhibitors, HIV-1 RT inhibitors (such as Tenofovir, epivir, zidovudine, or stavudine, and the like), HIV-1 protease inhibitors (such as saquinavir, ritonavir, nelfmavir, indinavir, amprenavir, lopinavir, atazanavir, tipranavir, fosamprenavir, and the like), HIV-1 fusion inhibitors (such as Fuzeon (T20), or PRO-542, SCH-C, and the like), polybiguanides (PBGs), herpes virus DNA polymerase inhibitors (such as acyclovir, ganciclovir, cidofovir, and the like), herpes virus protease inhibitors, herpes virus fusion inhibitors, herpes virus binding inhibitors, and ribonucleotide reductase inhibitors.

The present invention also provides methods for the treatment or prevention of a virus, bacterial, or fungal infection in a host, which comprises administering to the host a therapeutically effective amount of an anionic acrylic based polymer, a prodrug thereof, or a pharmaceutically acceptable salt of said anionic acrylic based polymer or prodrug in combination with one or more anti-infective agents. More particularly, the one or more anti-infective agents can be an anti-viral agent, an anti-bacterial agent, an anti-fingal agent, or combination thereof. More particularly, the anionic acrylic based polymer and the one or more anti-infective agents can be administered simultaneously or sequentially.

In another embodiment, said one or more anti-infective agents of such methods include antiviral protease enzyme inhibitors (PI), virus DNA or RNA or reverse transcriptase (RT) polymerase inhibitors, virus/cell fusion inhibitors, virus integrase enzyme inhibitors, virus/cell binding inhibitors, and/or virus or cell helicase enzyme inhibitors, bacterial cell wall biosynthesis inhibitors, virus or bacterial attachment inhibitors, HIV-1 RT inhibitors (such as Tenofovir, epivir, zidovudine, or stavudine, and the like), HIV-1 protease inhibitors (such as saquinavir, ritonavir, nelfmavir, indinavir, amprenavir, lopinavir, atazanavir, tipranavir, fosamprenavir, and the like), HIV-1 fusion inhibitors (such as Fuzeon (T20), or PRO-542, SCH-C, and the like), polybiguanides (PBGs), herpes virus DNA polymerase inhibitors (such as acyclovir, ganciclovir, cidofovir, and the like), herpes virus protease inhibitors, herpes virus fusion inhibitors, herpes virus binding inhibitors, and ribonucleotide reductase inhibitors.

The present invention also provides pharmaceutical combination compositions comprising a therapeutically effective amount of a composition which comprises a therapeutically effective amount of an anionic cellulose-based polymer, a prodrug of said anionic cellulose based polymer, or a pharmaceutically acceptable salt of said anionic cellulose-based polymer or prodrug; one or more anti-infective agents; and a pharmaceutically acceptable carrier, vehicle or diluent.

In another embodiment, said one or more anti-infective agents in such pharmaceutical combination compositions include antiviral protease enzyme inhibitors (PI), virus DNA or RNA or reverse transcriptase (RT) polymerase inhibitors, virus/cell fusion inhibitors, virus integrase enzyme inhibitors, virus/cell binding inhibitors, and/or virus or cell helicase enzyme inhibitors, bacterial cell wall biosynthesis inhibitors, virus or bacterial attachment inhibitors, HIV-1 RT inhibitors (such as Tenofovir, epivir, zidovudine, or stavudine, and the like), HIV-1 protease inhibitors (such as saquinavir, ritonavir, nelfinavir, indinavir, amprenavir, lopinavir, atazanavir, tipranavir, fosamprenavir, and the like), HIV-1 fusion inhibitors (such as Fuzeon (T20), or PRO-542, SCH-C, and the like), polybiguanides (PBGs), herpes virus DNA polymerase inhibitors (such as acyclovir, ganciclovir, cidofovir, and the like), herpes virus protease inhibitors, herpes virus fusion inhibitors, herpes virus binding inhibitors, and ribonucleotide reductase inhibitors.

The present invention also provides pharmaceutical combination compositions comprising a therapeutically effective amount of a composition which comprises a therapeutically effective amount of an anionic acrylic-based polymer, a prodrug of said anionic acrylic-based polymer, or a pharmaceutically acceptable salt of said anionic cellulose based polymer or prodrug; one or more anti-infective agents; and a pharmaceutically acceptable carrier, vehicle or diluent.

In another embodiment, said one or more anti-infective agents in such pharmaceutical combination compositions include antiviral protease enzyme inhibitors (PI), virus DNA or RNA or reverse transcriptase (RT) polymerase inhibitors, virus/cell fusion inhibitors, virus integrase enzyme inhibitors, virus/cell binding inhibitors, and/or virus or cell helicase enzyme inhibitors, bacterial cell wall biosynthesis inhibitors, virus or bacterial attachment inhibitors, HIV-1 RT inhibitors (such as Tenofovir, epivir, zidovudine, or stavudine, and the like), HIV-1 protease inhibitors (such as saquinavir, ritonavir, nelfinavir, indinavir, amprenavir, lopinavir, atazanavir, tipranavir, fosamprenavir, and the like), HIV-1 fusion inhibitors (such as Fuzeon (T20), or PRO-542, SCH-C, and the like), polybiguanides (PBGs), herpes virus DNA polymerase inhibitors (such as acyclovir, ganciclovir, cidofovir, and the like), herpes virus protease inhibitors, herpes virus fusion inhibitors, herpes virus binding inhibitors, and ribonucleotide reductase inhibitors.

The present invention also provides kits comprising:

(a) an anionic cellulose-based polymer, a prodrug of said anionic cellulose-based polymer, or a pharmaceutically acceptable salt of said anionic cellulose based polymer or prodrug;

(b) optionally one or more anti-infective agents;

(c) a pharmaceutically acceptable carrier, vehicle or diluent; and

(d) a container for containing said polymer and anti-infective agent of (a) and (b), respectively; wherein said polymer and anti-infective agent are present in amounts efficacious to provide a therapeutic effect. Preferably, both the polymer and the anti-infective agent are present in unit dosage form.

More particularly, the one or more anti-infective agents in such kits can be an anti-viral agent, an anti-bacterial agent, an anti-fungal agent, or the combination thereof.

The present invention also provides a kit comprising:

(a) an acrylic-based polymer, a prodrug of said acrylic-based polymer, or a pharmaceutically acceptable salt of said anionic cellulose based polymer or prodrug;

(b) optionally one or more anti-infective agents;

(c) a pharmaceutically acceptable carrier, vehicle or diluent; and

(d) a container for containing said polymer and anti-infective agent of (a) and (b), respectively; wherein said polymer and anti-inactive agent are present in amounts efficacious to provide a therapeutic effect. It is preferred that the polymer and anti-infective agent are present in unit dosage form.

More particularly, the one or more anti-infective agents in such kits can be an anti-viral agent, an anti-bacterial agent, an anti-fungal agent, or the combination thereof. It is to be understood that in an embodiment of the present invention, the various kits within the scope of the present invention can comprise a polymer of Formula I and a polymer of Formula II, or two or more polymers of Formula I or two or more polymers of Formula II.

The present invention also provides a vehicle or an adjuvant of a therapeutic or cosmetic composition comprising a polymer having a repeating unit of the following 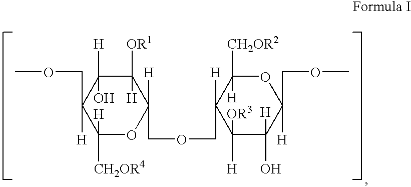
or pharmaceutically acceptable salts thereof;
wherein R¹, R², R³, and R⁴ are the same or different, and are defmed as hereinabove, i.e., wherein each R¹, R², R³, and R⁴ are the same or different, and are hydrogen, C₁-C₆ hydroxyalkyl, an aliphatic group, preferably C₁-C₆ alkyl, an alicyclic group, an aryl group, or arylaliphatic or an heteroring group or 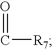
   wherein each of said aliphatic group, alicyclic group, aryl group, and heteroring group is independently unsubstituted or substituted by one or more substituents selected from the group consisting of carboxylic acid, sulfuric acid, sulfonic acid, carboxylate, sulfate, sulfonate, and acidic anhydride; R⁷ is hydrogen, C₁-C₆ hydroxyalkyl, an aliphatic group, preferably C₁-C₆ alkyl, alicyclic group, an aryl group, an arylaliphatic group, or an heteroring group, which aliphatic groups, alicyclic groups, aryl group and heteroring are independently unsubstituted or substituted by one or more substituents selected from carboxylic acid, sulfonic acid, sulfonic acid carboxylate, sulfate, sulfonate and acidic anhydride, however, at least one of R¹, R², R³ and R⁴ contains at least one COOH group, wherein the pKa of one of the COOH groups present or if its salt is present, the corresponding acid, is less than about 5.0.

The present invention also provides a thickener for topical administration of a therapeutic or cosmetic composition comprising a polymer having a repeating unit of the following formula: 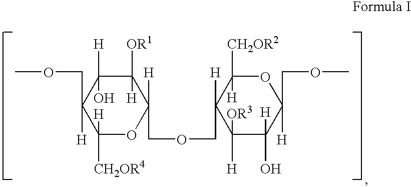
or pharmaceutically acceptable salts thereof,
R¹, R², R³, and R⁴ are the same or different, and are defmed as hereinabove, i.e., wherein each R¹, R², R³, and R⁴ are the same or different, and are hydrogen, C₁-C₆ hydroxyalkyl, an aliphatic group, preferably C₁-C₆ alkyl, an alicyclic group, an aryl group, an arylaliphatic or an heteroring group or 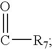
   wherein each of said aliphatic group, alicyclic group, aryl group, and heteroring group is independently unsubstituted or substituted by one or more substituents selected from the group consisting of carboxylic acid, sulfuric acid, sulfonic acid, carboxylate, sulfate, sulfonate, and acidic anhydride; R⁷ is hydrogen, C₁-C₆ hydroxyalkyl, an aliphatic group, preferably C₁-C₆ alkyl, alicyclic group, an aryl group, an arylaliphatic or an heteroring group, which aliphatic groups, alicyclic groups, aryl group and heteroring are independently unsubstituted or substituted by one or more substituents selected from carboxylic acid, sulfonic acid, sulfuric acid, carboxylate, sulfate, sulfonate and acidic anhydride, however, at least one of R¹, R², R³ and R⁴ contains at least one COOH group, wherein the pKa of one of the COOH groups is present or if its salt is present, the corresponding acid is less than about 5.0.

The present invention also provides a vehicle or an adjuvant of a therapeutic or cosmetic composition comprising a polymer having a repeating unit of the following formula: 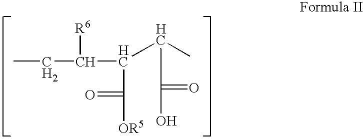
or pharmaceutically acceptable salts thereof;
wherein each R⁵ is hydrogen, an aliphatic group , an alicyclic group, an aryl group, arylaliphatic or an heteroring group; wherein each of said aliphatic group , alicyclic group, aryl group, or heteroring group is independently unsubstituted or substituted by an aliphatic group, alicyclic group, an aryl or aryl aliphatic or R⁵ is 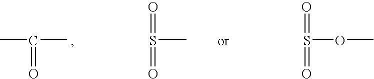
   which is bonded to an aliphatic group, aryl group, alicyclic group or arylaliphatic or heteroring, all of which may be unsubstituted or substituted by one or more substituents chosen from the group consisting of carboxylic acid, sulfuric acid, sulfonic acid, carboxylate, sulfate, sulfonate, and acidic anhydride; and each R⁶ is hydrogen, C₁-C₆ alkyl or C₁-C₆ hydroxyalkyl aryl or SR⁸ or OR⁸, wherein each R⁸ is hydrogen, aliphatic group, alicyclic group, aryl group, or heteroring which may be unsubstituted or substituted with an aliphatic group, alicyclic group or aryl group, or aryl aliphatic group or aliphatic aryl group.

The present invention also provides a thickener for topical administration of a therapeutic or cosmetic composition comprising a polymer having a repeating unit of the following formula: 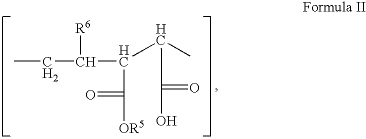
or pharmaceutically acceptable salts thereof;
wherein each R⁵ is hydrogen, an aliphatic group , an alicyclic group, an aryl group, arylaliphatic or an heteroring group; wherein each of said aliphatic group , alicyclic group, aryl group, or heteroring group is independently unsubstituted or substituted by an aliphatic group, alicyclic group, an aryl or aryl aliphatic or aliphatic aryl group or R⁵ is 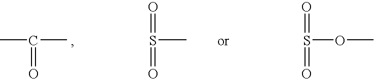
   which is bonded to an aliphatic group, aryl group, alicyclic group or arylaliphatic or heteroring, all of which may be unsubstituted or substituted with an aliphatic group, aryl group, alicyclic group, or an arylaliphatic or heteroring which groups may be unsubstituted or substituted by one or more substituents chosen from the group consisting of carboxylic acid, sulfuric acid, sulfonic acid, carboxylate, sulfate, sulfonate, and acidic anhydride; and each R⁶ is hydrogen, C₁-C₆ alkyl or C₁-C₆ hydroxyalkyl aryl or SR⁸OR⁸, wherein each R⁸ is hydrogen, aliphatic group, alicyclic group, aryl group, or heteroring which may be unsubstituted or substituted with an aliphatic group, alicyclic group or aryl group, or aryl aliphatic group or aliphatic aryl group.

### BRIEF DESCRIPTION OF THE DRAWINGS

FIG. 1 depicts graphically the cytotoxicity evaluation of various anionic cellulose based polymers in HeLa derived P4-CCR5 cells. Effect of varying doses of HPMCT (hydroxylpropyl methyl cellulose trimellitate), HPMCP (hydroxypropyl methyl cellulose phthalate), CAP (cellulose acetate phthalate, and CAT (cellulose acetate trimellitate) on uninfected P4-CCR5 cells are shown in FIG. 1. In this experiment, test cells were exposed to HPMCT, HPMCP, CAP, or CAT, or the control compound Dextran Sulfate (DS) for two hours at 37° C. in 5% CO₂ atmosphere in tissue culture media. This is the standard amount of exposure that cells will receive in viral binding inhibition (VBI) efficacy assays, like those shown in FIGS. 2 and 3 hereinbelow. After drug exposure, cells were washed and incubated in fresh, drug-free medium for 48 hrs at 37° C. in 5% CO₂ atmosphere at which time the cells were assessed for viability using the MTT tetrazolium dye as described by Rando et al. (“Suppression of Human Immunodeficiency virus type 1 activity in vitro by oligonucleotides which form intramolecular tetrads”, J. Biol. Chem. 270:1754-1760 (1995)), the contents of which are incorporated by reference.

FIG. 2 depicts graphically the inhibitory effect of HPMCT, HPMCP, CAP, CAT, and the control compound DS on HIV-1IIIB, a CXCR4 tropic strain of HIV-1. Viral binding inhibition (VBI) assays were performed using P4-CCR5 cells treated with differing concentrations of cellulose-based anionic polymer, or the control compound DS, for two hours in the presence of CXCR4 tropic HIV-1IIIB. The cells were then washed and incubated at 37° C. in drug- and virus-free media for 48 hrs. At the end of the 48 hr culture, the intracellular production of β-galactosidase (β-gal) was measured as described by Ojwang et al. (“T30177, an oligonucleotide stabilized by an intramolecular guanosine octet, is a potent inhibitor of laboratory strains and clinical isolates of human immunodeficiency virus type 1.” Antimicrobial Agents and Chemotherapy 39:2426-2435 (1995)), the contents of which are incorporated by reference. The decrease in β-gal production was measured relative to control infected but untreated cells.

FIG. 3 depicts graphically the effect of HPMCT on the CCR5 tropic HIV-1 strain BaL. In this VBI assay, the P4-CCR5 target cells treated with differing concentrations of HPMCT or the control compound DS for two hours in the presence of CCR5 tropic HIV-1BaL. The cells were then washed and incubated at 37° C. in drug and virus-free media for 48 hrs. At the end of the 48 hr culture, the intracellular production of β-gal was measured as described by Ojwang et al. (“T30177, an oligonucleotide stabilized by an intramolecular guanosine octet, is a potent inhibitor of laboratory strains and clinical isolates of human immunodeficiency virus type 1.”Antimicrobial Agents and Chemotherapy 39:2426-2435 (1995)), the contents of which are incorporated by reference. The decrease in β-gal production was measured relative to control infected but untreated cells.

FIG. 4 depicts graphically the results obtained using HPMCT in a cell free virus inhibition (CFI) assay. In this CFI assay 8×10⁴ P4-CCR5 cells were plated in 12-well plates 24 hr prior to the assay. On the day of the assay, 5 μl of serially diluted compound, either control (DS) or HPMCT, was mixed with an equal volume of HIV-1IIIB (approximately 10⁴-10⁵ tissue culture infectious dose₅₀ (TCID₅₀) per ml) and incubated for 10 minutes at 37° C. After the incubation period, the mixture was diluted (100-fold in RPMI 1640 media including 10% FBS), and aliquots were added to duplicate wells at 450 μl per well. After a 2-hr incubation period at 37° C., an additional 2 ml of new media was added to the cells. At 46 hr post-infection at 37° C., the cells were washed twice with phosphate buffered saline (PBS) and lysed using 125 μl of a lysis buffer comprised of 100 mM potassium phosphate (pH 7.8), and 0.2% Triton X-100. HIV-1 infectivity (monitored by assaying for β-gal production) was measured by mixing 2-20 μl of centrifuged lysate with a reaction buffer comprised of Tropix 1,2-dioxetane substrate in sodium phosphate (pH 7.5), 1 mM MgCl₂ and 5% Sapphire II™ enhancer, incubating the mixture for 1 hr at RT (room temperature), and quantitating the subsequent luminescence using a luminometer.

FIG. 5 depicts graphically the combination studies using HPMCT and PEHMB (polyethylene hexamethylene biguanide). HPMCT was added over a range of concentrations combined with 0.01% PEHMB, (Catalone, B .J., et al. “Mouse model of cervicovaginal toxicity and inflammation for the preclinical evaluation of topical vaginal microbicides”, Antimicrob. Agents and Chemother. 1837-1847 (2004)), the contents of which are incorporated by reference, to P4-CCR5 cells in a VBI assay (FIG. 5A). Reverse experiments were also performed in which 0.0002% HPMCT was used in combination with various concentrations of PEHMB (FIG. 5B). In these assays a 1.0% wt/vol stock solutions of HPMCT dissolved in 20 mM sodium citrate buffer pH 5.0, and a 5% PEHMB wt/vol stock solution made up in saline were used.

FIG. 6 depicts graphically the effect of HPMCT in the cell-associated virus inhibition (CAI) assay. In this assay, SupT1 cells (3×10⁶) were infected with HIV-1IIIB in RPMI media (30 μgl) and incubated for 48 hr. Infected SupT1 cells were pelleted and then resuspended (8×10⁵ cells/ml) in tissue culture media. Differing concentrations of HPMCT (5 μl) were added to infected SupT1 cells (95 μl) and incubated for 10 min at 37° C. After incubation, the mixture was diluted in RPMI media (1:10), and 300 μl of the diluted mixture was added to appropriate wells in triplicate. In the wells, target P4-CCR5 cells were present. Production of infectious virus resulted in β-gal induction in the P4-CCR5 target cells. Plates were incubated (2 hr at 37° C.), washed (2×) with PBS, and then drug and virus-free media (2 ml) was added before further incubation (22-46 hr). Cells were then aspirated and washed (2×) and then incubated (10 min at room temperature) with lysis buffer (125 μl). Cell lysates were assayed for β-gal production utilizing the Galacto-Star™ kit (Tropix, Bedford, Mass.).

FIG. 7 depicts graphically the HSV-2 plaque reduction assay. HSV-2 (strain 333) virus stocks were prepared at a low multiplicity of infection with African Green monkey kidney (CV-1) cells, and subsequently cell-free supernatants were prepared from frozen and thawed preparations of lytic infected cultures. CV-1 cells were seeded onto 96-well culture plates (4×10⁴ cell/well) in 0.1 ml of minimal essential medium (MEM) supplemented with Earls salts and 10% heat inactivated fetal bovine serum and pennstrep (100 U/ml penicillin G, 100 mg/ml streptomycin sulfate) and incubated at 37° C. in 5% CO₂ atmosphere overnight. The medium was then removed and 50 ml of medium containing 30-50 plaque forming units (PFU) of virus diluted in test medium and various concentrations of HPMCT were added to the wells. Test medium consisted of MEM supplemented with 2% FBS and pennstrep. The virus was allowed to adsorb onto the cells in the presence of HPMCT for 1 hr. The test medium was then removed, and the cells were rinsed three times with fresh medium. A fmal 100 ml aliquot of test medium was added to the cells which were then further cultured at 37° C. Cytopathic effect was scored 24 to 48 hrs post infection when control wells showed maximum effect of virus infection. Each datum in FIG. 7 represents an average for at least two plates.

FIG. 8 depicts graphically the ability of acrylic copolymers and HPMCT to inhibit the growth of Neisseris gonorrhoeae (NG). Compounds were assessed in vitro for bacteriocidal activity against the F62 (serum-sensitive) strain of NG. NG colonies from an overnight plate were collected and resuspended in GC media at ˜0.5 OD600. Following 1:10,000 dilution, warm GC media were combined with compounds (10 microliters) in 96-well plates to achieve fmal compound concentrations. After incubation in a shaker incubator for 30 to 90 minutes at 37° C., aliquots were removed from each well, diluted 1:10 in media, and spotted on plates in duplicate. Colonies were counted after overnight incubation. In these assays, a 0.1% solution of the control compound polyhexamethylene bis biguanide (PHMB or Vantocil) and the alternating copolymer of polystyrene with maleic anhydride were able to completely inhibit the growth of NG F62 even with exposure times as short as 30 min. The acrylic copolymer consisting of methylvinyl ether and maleic anhydride (MVE/MA) was moderately effective at inhibiting NG growth under these conditions with the best inhibition (˜75%) occurring after a 90 minute exposure of drug to bacteria. HPMCT was less effective; though after a 90 min exposure of drug to NG F62, the inhibition of bacterial growth was significant (˜55%).

FIG. 9 depicts graphically the effect of pH on the solubility of the cellulose-based polymers CAP and HPMCT. In this experiment, the degree of HPMCT (0.038% in 1 mM sodium citrate buffer, pH 7) or CAP (0.052% in 1 mM sodium citrate buffer, pH 7) in solution was monitored using ultraviolet absorbance. CAP was monitored at 282 nm, and HPMCT was monitored using 288 nm u.v. light. The samples were slowly made more acidic by the gradual addition of 0.5N HCl. After each addition, the pH was determined, and the samples were vortexed for five seconds and then centrifuged using a tabletop centrifuge at 3000 rpm for five minutes. The supernatant was then collected and monitored for the presence of polymer using the absorbance conditions described hereinabove. The results from this experiment are as predicted by the pKa values of the remaining dissociable carboxylic acid groups of the trimellityl and phthalate moieties on the cellulose backbone, in that HPMCT stays in solution at lower pH than CAP.

FIG. 10 illustrates graphically the effect of pH on solubility and dissociation of phthalic and trimellitic acid-containing cellulose polymers. HCl was slowly added to buffered polymer solutions of HPMCT or CAP. At each titration point the samples were centrifuged briefly and the polymer remaining in the supernatant was monitored as was the amount of carboxylic acid remaining dissociated. Then these data sets were combined to visualize the effect of pH on these two parameters.

FIG. 11 illustrates graphically the effect of pH on the antiviral efficacy of phthalic and trimellitic acid-containing cellulose polymers. Polymer samples were serially diluted and then placed in low pH conditions for a brief time before being rapidly neutralized by addition to well-buffered target cells. The assays were then performed by adding H9/HIV-1_{SKI} cells to the media. The effect of CAP (A) and HPMCT (B) on HIV-1 in this system was determined by monitoring intracellular p24 production 24 hr post-infection.

FIG. 12 shows graphically the effect of pH on the antiviral efficacy of phthalic and trimellitic acid-containing cellulose polymers in a CD4-independent infection assay. Polymer samples were serially diluted and then placed in low pH conditions for a brief time before being rapidly neutralized by addition to well-buffered ME180 cells. The assays were then performed by adding H9/HIV-1_{SKI} cells to the media. The effect of CAP (A), HPMCT (B) and DS (C) on HIV-1 transmission in this system was determined by monitoring extracellular p24 production 6 days post-infection.

FIG. 13 illustrates graphically the effect of HPMCT on virus infection in PBMCs. A CXCR4 tropic (CMU06), a CCR5 tropic (JRCSF) or a dual tropic (BR/92/014) strain of HIV was used to infect activated PBMCs. Seven days post-infection, cell-free supernatant samples were collected for analysis of reverse transcriptase activity. Cell viability was measured by addition of MTS to the cells at this time. The results of this experiment show that both CAP (A) and HPMCT-35 (B) are effective inhibitors of all three virus strains tested. The cytotoxicity observed after a seven day exposure of test compound to PBMCs was also plotted (C).

### DETAILED DESCRIPTION OF THE INVENTION

The term “acrylic”, as used herein, denotes derivatives of acrylic and methacrylic acid, including acrylic esters and compounds containing nitrile and amide groups as defined herein. Polymers based on acrylic are well known in the art and the term “acrylic based polymer” is well understood by one skilled in the art.

The term “cellulose”, as used herein, denotes a long-chain polysaccharide carbohydrate and derivatives thereof as described herein. Polymers based on cellulose are well known in the art and the term “cellulose based polymer” is well understood by one skilled in the art.

The term “monomer” refers to a repeating unit of the cellulose or acrylic polymer. In an embodiment, the monomer is a moiety of Formula I and II herein which forms part of the polymer and repeats itself, as described hereinbelow.

The expression “prodrug” refers to compounds that are drug precursors which, following administration, release the drug in vivo via some chemical or physiological process (e.g., a prodrug on being brought to the physiological pH or through enzyme action is converted to the desired drug form).

By “pharmaceutically acceptable” or synonym thereof, it is meant that the drug, carrier, vehicle, diluent, excipient and/or salt must be compatible with the other ingredients of the formulation, and not deleterious to the recipient thereof.

As used herein the term “aliphatic” is meant to refer to a hydrocarbon having 1 up to 10 carbon atoms linked in open chains. By “hydrocarbon”, it is meant an organic compound in which the main chain contains only carbon and hydrogen atoms; however, as defined herein, it may be optionally substituted by groups which contain other atoms. The term “aliphatic”, as used herein, includes C₁-C₁₀ alkyl, C₂-C₁₀ alkenyl, C₂-C₁₀ alkynyl, and C₄-C₁₀ alkenyl-alkynyl. It is preferred that the aliphatic group is C₁-C₆ alkyl, C₂-C₆ alkenyl, C₂-C₆ alkynyl, or C₄-C₈ alkenyl-alkynyl. It is more preferred that the aliphatic group is C₂-C₆ alkyl or C₂-C₆ alkenyl. It is to be noted that, as defined herein, the aliphatic group is attached directly to the oxygen atom in Formula I and Formula II. However, as described hereinbelow, the alkyl, alkenyl, alkynyl, or alkenyl-alkynyl group is further substituted, as defined herein.

As used herein the term “alicyclic” is meant to refer to a cyclic hydrocarbon that contains one or more rings of carbon ring atoms but is not aromatic. The term alicyclic as used herein includes completely saturated as well as partially saturated rings. The alicyclic group contains only carbon ring atoms and contains from 3 to 14 carbon ring atoms. The alicyclic group may be one ring, or it may contain more than one ring. For example, it may be bicyclic or tricyclic. It is preferred that the alicyclic group is monocyclic or bicyclic, and most preferably monocyclic. The alicyclic ring may contain one or two carbon-carbon double or triple bonds. If it contains any unsaturated carbon atoms in the ring, it is preferred that the alicyclic group contains one or two double bonds. However, as defined, the alicyclic group is not aromatic. It is preferred that the alicyclic group contains 3 to 10 carbon ring atoms and more preferably 5, 6, 7, or 8 ring carbon atoms. More preferably, it is a monocyclic ring containing 5, 6, 7, or 8 ring carbon atoms. Examples include cyclopropyl, cyclobutyl, cyclopentyl, cyclohexyl, cycloheptyl, cyclooctyl, cyclononyl, cyclodecanyl, adamantyl, norbomyl, cycloheptenyl, cycopentenyl, cyclohexenyl, 1,3-cyclopentadienyl, 1,3-cyclohexadienyl, 1,4-cyclohexadienyl, 1,3,5-cycloheptatrienyl, 1,4-cycloheptadienyl, 1,3-cycloheptadienyl and the like. It is more preferred that the alicyclic group is cyclopentyl, cyclohexyl, cycloheptyl, cyclooctyl, 1,3-cyclohexadienyl, or 3-cyclopentadienyl.

The term “aryl” as used herein refers to an optionally substituted six to fourteen membered aromatic ring, including polyaromatic rings. The aromatic rings contain only carbon ring atoms. It is preferred that the aromatic rings are monocyclic or fused bicyclic rings. Examples of aryl include phenyl, α-naphthyl, β-naphthyl, and the like.

The term “arylaliphatic” refers to aliphatic group, as defined herein, as a bridging group between an aryl group and the main chain. Examples include aryl lower alkyl, e.g. benzyl, phenethyl, naphthylmethyl and the like.

The term “heteroring” as used herein refers to an optionally substituted 5-, 6- or 7-membered heterocyclic ring containing from 1 to 3 ring atoms selected from the group consisting of an oxygen atom as part of a ring anhydride or lactam, and sulfur as part of S(O)m, wherein m is 1 or 2. The heteroring may be further fused to one or more benzene rings or heteroaryl rings, more preferably fused to one or more aromatic rings. By “heterocyclic ring” it is meant a closed ring of atoms of which at least one ring atom is not a carbon atom.

The term “C₁-C₁₀ alkyl” as used herein refers to an alkyl group containing one to ten carbon atoms. The alkyl group may be straight chain or branched. Examples include methyl, ethyl, propyl, isopropyl, butyl, sec-butyl, tert-butyl, pentyl, neopentyl, isopentyl, hexyl, heptyl, 2-methylpentyl, octyl, nonyl, decanyl, and the like.

The term lower alkyl refers to “C₁-C₆ alkyl”. As used herein, these terms refer to an alkyl group containing one to six carbon atoms. Examples of alkyl of one to six carbon atoms, inclusive, are methyl, ethyl, propyl, butyl, pentyl and hexyl and all isomeric forms and straight-chain and branched chain thereof.

The term “C₁-C₆ hydroxyalkyl” as used herein refers to alkyl of one to six carbon atoms which is further substituted by one or more hydroxyl groups.

The term “C₂-C₁₀ alkenyl” refers to an alkenyl group containing two to ten carbon atoms and containing one or more carbon-carbon double bonds. The alkenyl groups may be straight-chain or branched. Although it must contain one carbon-carbon double bond, it may contain two, three or more carbon-carbon double bonds. It is preferred that it contains 2, 3, or 4 carbon-carbon double bonds. Moreover, the carbon-carbon double bond may be unconjugated or conjugated if the alkenyl groups contain more than one carbon-carbon double bond. Preferably, the alkenyl group contains one or two carbon-carbon double bonds, and most preferably only one carbon-carbon double bond. Examples include ethenyl, propenyl, 1-butenyl, 2-butenyl, allyl, 1,3-butadienyl, 2-methyl-1-propenyl, 1,3-pentadienyl, 1,4-pentadienyl, 1-pentenyl, 2-pentenyl, 3-pentenyl, 1-hexenyl, 2-hexenyl, 3-hexenyl, 1,3-hexadienyl, 1,3,5-hexatrienyl, 1-heptenyl, 2-heptenyl, 3-heptenyl, 1-octenyl, 2-octenyl, 3-octenyl, 4-octenyl, 1-nonenyl, 1-decenyl, and the like. It is preferred that the C₂-C₁₀ alkenyl is a C₂-C₆ alkenyl group. In addition, it is most preferred that the alkenyl group is C₂-C₄ alkenyl group, and more preferably vinyl. It is also preferred that alkenyl group contains a carbon-carbon double bond that is at the one end of the carbon chain (1-position).

The term “C₂-C₁₀ alkynyl” refers to an alkynyl group containing two to ten carbon atoms and one or more carbon-carbon triple bonds. The alkynyl group may be straight-chained or branched. Although it must contain one carbon-carbon triple bond, it may contain 2, 3, or more carbon-carbon triple bonds. It is preferred that it contains 2, 3, or 4 carbon-carbon triple bond, and more preferably one or two carbon-carbon triple bond. Examples include ethynyl, propynyl, 1-butynyl, 2-butynyl, 1-pentynyl, 2-pentynyl, 3-methyl-1-butynyl, 1-hexynyl, 2-hexynyl, 3-hexynyl, 1,3-hexadiynyl, 1,3,5-hexatriynyl, 1,3-dibutdiynyl, 1,3-dipentadiynyl, and the like. It is preferred that the C₂-C₁₀ alkenyl contains two to six carbon atoms and more preferably two to four carbon atoms. It is most preferred that the alkenyl group is ethynyl. It is also preferred that alkenyl group contains a carbon-carbon double bond at the end of the carbon chain 1′ position.

The term “C₄-C₁₀ alkenyl-alkynyl” refers to a moiety comprised of two to ten carbon atoms containing at least one carbon-carbon double bond and at least one carbon-carbon triple bond. The preferred alkenyl-akynyl moieties contain at most two carbon-carbon double bonds and at most two carbon-carbon triple bonds. It is more preferred that it contains one or two carbon-carbon double bonds and one carbon-carbon triple bond, and most preferably one carbon-carbon double bond and one carbon-carbon triple bond.

The term “heteroaryl” refers to a heteroaromatic group containing five to fourteen ring atoms and at least one ring hetero atom selected from the group consisting of N, O, and S. When the heteroaryl group contains two or more ring hetero atoms, the ring hetero atoms may be the same or different. It is preferred that the heteroaryl group contains at most two ring hetero atoms. The heteroaryl group may be monocyclic or may consist of one or more fused rings. It is preferred that the heteroaryl group is monocyclic, bicyclic, or tricyclic, and more preferably monocyclic or bicyclic. It is most preferred that the heteroaryl group consists of a five or six membered heteroaromatic ring containing a ring heteroatom selected from the group consisting of oxygen, nitrogen, and sulfur which may be fused to one or more benzene rings, that is, benzyl fused heteroaryls. Examples include thienyl, furyl, pyridyl, pyrimidyl, benzofuran, pyrazole, indazole, imidazole, pyrrole, quinoline, and the like.

It is to be understood that the alkyl, alkenyl, alkynyl, alkenyl-alkynyl, alicyclic, heteroaryl, or heteroring groups may be optionally substituted further with one or more electron donating groups or electron withdrawing groups, both of which are terms that describe the ability of the moiety to donate or withdraw electrons compared to hydrogen. If the moiety donates electrons more than a hydrogen atom does, then it is an electron donating group. If the moiety withdraws electrons more than a hydrogen atom does, then it is an electron withdrawing group. Examples of electron donating and withdrawing groups include C₁-C₁₀ alkyl, aryl, carboxy, C₂-C₁₀ alkenyl, heterocyclic, C₂-C₁₀ aLkynyl, C₄-C₁₀ alkeynyl-alkynyl, C₁-C₁₀ aLkoxy, C₁-C₁₀ carbalkoxy, aryloxy, C₃-C₁₀ cycloalkoxy, formyl, C₂-C₁₀ alkylcarbonyl, mercapto, C₁-C₁₀ alkylthio, aryl(C₁-C₁₀)alkyl, aryl(C₁-C₁₀)alkoxy, halo, nitro, cyano, amino, C₁-C₁₀ alkylamino, C₂-C₂₀ dialkyl amino, and the like.

As used herein, the term “C₂-C₁₀ alkylcarbonyl” refers to an alkyl group containing two to ten carbon atoms in which the hydrogen of the CH₂ group is replaced with one or more carbonyl groups. Examples include formyl, acetyl, propionyl, and the like.

The term “heterocyclic” refers to a cyclic moiety containing three to ten ring atoms wherein at least one of the ring atoms is a heteroatom selected from the group consisting of S, O, and N. The heterocyclic moiety may contain one ring or more than one ring. If it contains more than one ring, the rings are fused, e.g. bicyclic, tricyclic, and the like. In addition, the heterocyclic may contain more than one ring heteroatoms, e.g. two, three, or four heteroatoms. If it contains more than one ring heteroatoms, those ring hetero- atoms can be the same or different. The heterocyclic as used herein include the benzyl fused heterocyclics, that is, aromatic ring fused to the heterocyclic ring, as well as heteroaryls. Examples include furyl, quinolyl, pyrrolyl, tetrahydrofuranyl, morpholinyl, thienyl, pyridyl, and the like.

The term “carboxylic acid” refers to one or more COOH groups or salt thereof or combination thereof. Thus, in one embodiment an aliphatic group, aromatic group, alicylic group or heteroring group may each be substituted by one or more —COOH groups or salts thereof or combination thereof. It is preferred that the cellulose polymer, such as that of Formula I contains at least one COOH group or salt thereof. In addition, the pKa of a COOH group therein, as defined herein, is less than about 5.

In an embodiment, the monomer of the cellulose polymer contains one, two or three —COOH groups.

In another embodiment, the acrylic based polymer such as a polymer having the repeating monomer unit of Formula II is substituted by one or more COOH-groups or salts thereof or combinations thereof. The various aliphatic groups, aromatic groups, alicylic groups or heteroring groups as defined for the cellulose based polymers and the acrylic based polymers may be further substituted as described hereinabove. It is preferred that the various R groups e.g. R₁-R₆, are further substituted by one or more hydroxyl groups. In the preferred embodiment, the alkyl- alkenyl- alkynyl-, and aryl, e.g., phenyl groups, are each substituted by one, two, or three —COOH groups.

The term “sulfuric acid” refers to one or more —OSO₃H or salts thereof, or combination thereof In an embodiment, an aliphatic group, aromatic group, alicylic group or heteroring group described hereinabove is substituted by one or more —OSO₃H groups or salts thereof It is preferred that if present, the various R groups are substituted by one, two, or three —OSO₃H groups. The various aliphatic group, aromatic group, alicylic group or heteroring groups may be further substituted as described hereinabove. In an embodiment, when the sulfuric acid group is present on a substituent, on an R group, the substitutent is also substituted by one or more hydroxy groups. It is preferred that alkyl, alkenyl, alkynyl, and aryl, e.g., phenyl, are each substituted by one, two, or three —OSO₃H groups.

The term “sulfonic acid” refers to one or more SO₃H or salt thereof or combination thereof. In one embodiment, an aliphatic group, aromatic group, alicylic group or heteroring group is substituted by one or more —SO₃H group or salt thereof or combination thereof. It is preferred that if present, the various R groups contain one, two, or three —SO₃H groups. The various aliphatic groups, aromatic groups, alicylic groups or heteroring groups may be further substituted as described hereinabove. It is preferred that when the R groups are substituted by a sulfonic acid group, they are further substituted by one or more hydroxyl groups. In an embodiment, the alkyl, alkenyl, alkynyl, and aryl, e.g., phenyl, are each substituted by one, two, or three —SO₃H groups.

The terms “carboxylate” refers to —COO⁻ group, while the “sulfonate” refers to —SO₃⁻ group, and the “sulfate” refers to —SO₃⁻ group.

The term “acid anhydride” as used herein refers to an anhydride formed by dehydration of two or more carboxylic acids, as defined herein, containing one to ten carbon atoms or one that forms an acid upon hydration; if bimolecular, said anhydride can be composed of two molecules of the same acid, or it can be a mixed anhydride. The carboxylic acids used to form an acid anhydride may be the same or different. The acid as used and the anhydride thus formed may be aliphatic, alicyclic, aryl, heteroaryl, heterocyclic or heteroring. As used herein, the anhydride may be unsubstituted or optionally substituted, as defined hereinabove.

The term “anti-infective agent” as used herein, refers to an agent capable of killing infectious pathogens or preventing them from spreading and causing infection. The infectious pathogens include viruses, bacteria, and fungi.

As used herein, the term “host” denotes any mammal. By “mammal” it is meant to refer to all mammals, including, for example, primates such as humans and monkeys. Examples of other mammals included herein are rabbits, dogs, cats, cattle, goats, sheep and horses. Preferably, the mammal is a female or male human.

The term, “therapeutically effective amount” or synonym thereto as defined herein, is that amount of the compounds described herein sufficient to effect beneficial or desired results, including clinical results. For example, when referring to an agent that inhibits viral, bacterial or fungal infection, a therapeutically effective amount of the compounds described herein is that amount sufficient to achieve reduction in the viral, bacterial or fungal infection as compared to the response obtained in the absence of (or without administering) the compound.

As used herein, and as well understood in the art, “treatment” is an approach for obtaining beneficial or desired results including clinical results. Beneficial or desired clinical results can include, but are not limited to, alleviation or amelioration of one or more symptoms or conditions, diminishment of extent of disease or infection by virus, fungus or bacteria, stabilized (i.e., not worsening) state of disease or infection by virus, fungus or bacteria, preventing spread of disease or infection by virus, fungus or bacteria, delay or slowing of disease progression or infection by virus, fungus or bacteria, amelioration or palliation of the disease state or infection by virus, fungus or bacteria, and remission (whether partial or total) whether detectable or undetectable or inhibiting or suppressing the infection by a virus, bacteria or fungus. “Treatment” can also mean prolonging survival as compared to expected survival if not receiving treatment.

Palliating” a disease or disorder or infection means that the extent and/or undesirable clinical manifestations of a disorder or a disease state or infection by bacteria, fungus or virus are lessened and/or time course of the progression is slowed or lengthened, as compared to not treating the disorder.

The term “modulate”, as used herein, includes the inhibition or suppression of a function or activity as well as the enhancement of a function or activity.

To “inhibit” or “suppress” or “reduce” a function or activity, such as viral, fungal or bacteria, is to reduce the function or activity when compared to otherwise same conditions, or alternatively, as compared to another condition.

The term “prophylaxis” as used herein refers to reduction in the risk or likelihood of development of infection from a virus, fungus or bacteria or reduction in the risk or likelihood of development of a disease caused by or associated with an infection of viral bacteria or fungus when a compound described herein is administered to a subject relative to the absent of (or without administrating) said compound to the subject.

The phrase “compound(s) of the present invention” or “polymer(s) of the present invention” or synonym(s) thereto shall at all times be understood to include both anionic cellulose based polymers and acrylic based polymers including compounds of Formula I and Formula II, including, for example, the free form thereof, e.g., the free acid or base form, and also, all prodrugs, polymorphs, hydrates, solvates, tautomers, and the like, and all pharmaceutically acceptable salts, unless specifically stated otherwise. It will also be appreciated that suitable active metabolites of such compounds are within the scope of the present invention.

The phrase “molecularly dispersed” as used herein means soluble in a particular solvent, such as water or other aqueous solvent. By soluble, it is meant that at least one gram of the compound dissolves in 100 mL of water or aqueous solvent.

The phrase “dissociated” as used herein means that the compound dissociates into its cationic or anionic form when placed in water or aqueous solvent at 25° C. or in heated water or aqueous solvent. The term “mostly dissociated” refers to at least 50% by weight of the compound or polymer that is present is dissociated into water or aqueous at 25° C. or in heated water or aqueous solvent into its anionic and cationic form.

The present invention relates to the use of anionic cellulose-based polymers, copolymers, and oligomers, and anionic acrylic-based polymers, copolymers, and oligomers. One preferred use thereof is for the treatment and prevention of infectious organisms, in particular, the infectious organisms causing STDs. In an embodiment, said anionic cellulose based polymers, copolymers, and oligomers are compounds of Formula I.

As defined herein, the backbone of the sugar moiety in Formula I and the acrylic moiety in Formula II are repeated. However, as defined, with respect to Formula I, each R¹ in each repeating unit and each R² in each repeating unit, each R³ in each repeating unit and each R⁴ in each repeating unit may be the same or different. Thus, each time the monomer depicted in Formula I is repeated, R¹ can be the same or different each time, R² be the same or different each time, R³ can be the same or different each time and R⁴ can be the same each time. Thus, the polymer of Formula I may contain more than one monomer unit, wherein the definition of each R¹, R², R³ and R⁴ may be the same or different from one monomer unit to the next. However, in an embodiment the polymer is comprised of one monomer wherein the definition of each R¹, R², R³ and R⁴ in each monomeric unit does not vary from monomer to monomer, i.e., in all the monomeric units, each R¹ is the same, each R² is the same, each R³ is the same can each R⁴ is the same, but R¹, R², R³ and R⁴ reflective to each others may be the same or different.

In an embodiment, at least one of R¹, R², R³ and R⁴ is an aryl group substituted by one, two or three carboxylic acids and optionally a sulfonic or sulfonic acid moiety or they are bonded to a 5 or 6 membered ring containing an 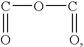
wherein the ring contains an oxygen ring atom and two acyl carbon ring atoms or 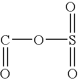
wherein the ring contain an oxygen ring atom, a S ring atom and an acyl carbon ring atom. Alternatively, at least one of R¹, R², R³ or R⁴ is an alkyl group or an alkenyl group containing one or two double bonds, wherein the alkyl group and alkenyl groups are bonded to one, two or three COOH groups. Moreover, each of these preferred embodiments can be further substituted by one or two or three hydroxyl groups. Many of the most preferred groups are depicted in Table 1, except the last entry therein. However, as stated hereinabove, the compounds of Formula I must contain at least one COOH group or salt thereof.

In another embodiment of the present invention, the groups containing the carboxylic acid moiety, sulfonic acid moiety or the sulfuric acid moiety, such as the groups depicted in the previous paragraph are present on R¹, R², R³ or R⁴. In another embodiment, these groups are present on R¹ and R².

The preferred group for R¹ and R² is independently Rg (COOR¹⁰)m wherein R⁹ is an aryl group such as phenyl and R¹⁰ is H or lower alkyl, which may be straight branched or branched, and m is 1, 2 or 3.

The preferred groups for R³ and R⁴ are independently the groups depicted in Table 1 or lower alkyl groups especially C₁-C₃ alkyl or hydrogen.

As defined hereinabove, the compounds of Formula I having carboxy groups thereon are prepared by reacting cellulose derivatives such as, e.g., cellulose or hydoxypropylmethyl cellulose with a carboxylic acid or acylating derivative thereof, such as the anhydride, or acid halide and the like under ester forming conditions. It is noted that the percentage of the polymer that is esterified is dependent upon the molar ratio of cellulose derivative to carboxylic acid acylating derivative and/or the reaction time. For example, HPMCT-29, HPMCT-35, HPMCT-41, HPMCT-49 are described hereinbelow, indicating that it is a hydroxypropyl methyl cellulose modified with either 29%, 35%, 41%, or 49% trimellitic acid by mole per mole of binary 1-4 linked glucose dimer (one repeat unit), i.e. per mole of repeating monomer unit of Formula I. In an embodiment of Formula I, it is preferred that the carboxylic acid moiety is present from about 40% to about 50% by mole per mole of binary 1-4 linked glucose dimer, i.e., per mole of repeating monomeric unit of Formula I.

The sulfuric acid and sulfonic acid derivatives are prepared by reacting cellulose or derivative thereof, as defined herein with a sulfonic acid or sulfuric acid derivative under conditions sufficient to form the sulfonate or sulfate.

Thus, in one embodiment of the present invention, the compound of Formula I contains the carboxylic acid derivative, the sulfuric acid derivative, or the sulfonic acid derivative in amounts ranging from about 40% to about 50% by mole per mole of each six membered ring moiety, e.g. glucose.

It is understood by one of ordinary skill in the art that the products of the reactions described hereinabove, are esters, sulfonates or sulfates. That is, the hydroxyl group of the cellulose react with the carboxylic acid, sulfuric acid or sulfonic acid moiety under conditions as to form the ester 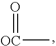
the sulfate 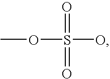
or the sulfonate

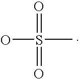

In these reactions, some of the monomeric units may not contain a carboxy group, or a sulfate or sufonate group, but this is minimized. However, the compounds utilized in the present invention contemplate the use of polymers where some of the monomer units do not contain one of those groups. Nevertheless, it is preferred that this occurs less than about 10% per mole of product, less than about 5% by mole and most preferably, less than about 1% by mole of the product.

Another aspect of the present invention is that at least one COOH group present thereon has a pKa of less than about 5.0 and more preferably ranges from about 1 to about 5, and most preferably 3 to 5. The present invention also contemplates the use of the corresponding salt. In other words, the compound of Formula I remains molecularly dispersed and mostly dissociated in aqueous solutions at a pH of less than 3, i.e., from pH 3 to pH 14, and most preferably at pH's ranging from about 3 to about 5.

As defined hereinabove, the compounds of Formula I are polymers comprised of two sugars having a 1, 6 linkage between the sugar moieties. The linkage is either an α or β linkage. However, it is preferred that the linkage is as shown in Formula I. Each of the sugar moieties is substituted by hydrogen, hydroxy, OR¹, OR³, CH₂OR², or CH₂OR⁴ as defined hereinabove. Furthermore, in a preferred embodiment, the polymers of Formula I are soluble in aqueous solutions at a pH ranging between about 3 to about 14. In another embodiment at least one of the R¹, R², R³ and R⁴ is not hydrogen, C₁-C₆ alky, or C₁-C₆ hydroxy alkyl.

In an embodiment, said anionic acrylic based polymers, copolymers, and oligomers are compounds of Formula II. In an embodiment of Formula II, R⁶ is OR⁸ or alkyl, hydroxy alkyl or aryl, e.g. phenyl, which R⁶ group is unsubstituted or substituted with an aliphatic, alicyclic group, aryl or aryl aliphatic group, as these groups are defined herein, which optionally may be further substituted by hydroxy and or halide. In another embodiment, R⁵ is hydrogen, aliphatic group, especially C₁-C₆ alkyl, alicylic group, aryl group or aryl aliphatic, although it is preferred that R⁵ is hydrogen or aliphatic group, especially C₁-C₆ alkyl. The R⁵ group, however, may also be a carboxyl group, or a 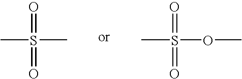
group, wherein 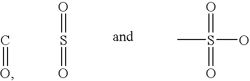
groups are bonded to by a bond to an aliphatic group, aryl group, alicyclic group, arylalicyclic or heteroring which may be unsubstituted or substituted by one or more carboxylic acid moiety, sulfiric acid moiety, sulfonic acid moiety and optionally with hydroxy or halide. In an embodiment, R⁵ has the same definition as R¹ defined above. Again, the preferred groups that can react with the acrylate polymer are depicted hereinbelow in Table 1 in accordance with the procedure described hereinbelow. There is no requirement that the pKa of a group on the acrylic based polymers including that of Formula II is less than about 5.0. Nevertheless, the pKa of a group thereon may be less than about 5.0 and can range from about 1 to 5 and more preferably from about 3 to about 5.

The polymer of Formula II is prepared by reacting the acrylic polymer with a carboxylic acid or acylating derivative thereof, sulfonic acid derivative or sulfuric acid derivative under effective conditions to form a compound of Formula II.

The R⁵ and each R⁶ moiety in each monomeric unit can be the same or different. Thus, each time a monomer depicted in Formula II is repeated, R⁵ can be the same or different and R⁶ can be the same or different. Thus, the polymer comprised of the monomer of Formula II can contain more than one monomeric unit wherein the definitions of each R⁵ and R⁶ are as indicated hereinabove. However, in an embodiment, the polymer is comprised of one monomer, wherein the definition of R⁵ and R⁶ in each monomeric unit does not vary although R⁵ and R⁶ relative to one another may be the same or different.

The monomeric unit (repeating unit) of Formula I preferably repeats (n+(x/2)) times, wherein n is an integer of 1 or greater and x is zero or 1. If the repeating unit of Formula I repeats one half time, it is meant that the polymer repeating unit ends at the oxygen atom separating one of the sugar moieties from the other. However, it is more preferred that the repeating unit of Formula I repeats n times and more preferably from 1 to about 600 times and more preferably from 1 to about 150 times and most preferably from about 100 to about 150 times. It is preferred that the cellulose polymer including the cellulose polymer of Formula I has a molecular weight ranging from about 350 to about 250,000 daltons and more preferably from about 350 to about 60,000 daltons and most preferably from about 35,000 to about 60,000 daltons.

The repeating unit in the acrylic polymer, including the monomer of Formula II repeats itself Z times, wherein Z is an integer of 1 or greater. It is preferred that Z is an integer ranging from 1 to about 10,000, and more preferably from 1 to about 6,000, and even more preferably from about 5 to about 1,000 and most preferably from about 5 to about 550. The molecular weight of the acrylic polymer, including the polymer of Formula II ranges from about 220 to about 2,000,000 and more preferably from about 1,000 to about 230,000 and most preferably from about 1,000 to about 130,000 daltons.

The compounds of the present invention include polymers having repeating units of Formula I and Formula II, and preferably have molecular weights greater than about 500 daltons. It is even more preferred that the molecular weight ranges from about 500 daltons to 2 million (MM) Daltons or higher. Further, the compounds of the invention described herein can also be chemically cross-linked by varying degrees to improve their linear viscoelastic properties.

The molecular weight of the polymers of Formula I and II, such as HPMCT and derivatives thereof, as defined herein, is important to its function in the biological system, especially with respect to the use in preventing or treating STDs. Without wishing to be bound, it is believed that lower molecular weight polymers, such as those of 10 kD to 15 kD, have higher diffusivity and faster transport to the infection site compared to the corresponding higher molecular weight polymers, such as about 50 kD. Since the higher molecular weight polymers are easier to formulate as gels or creams or the like, a mixture of lower and higher molecular weight polymers are useful to satisfy both the biological and delivery functions. Thus, the molecular weight distribution of the polymers should be considered in any application based on HPMCT or other polymer of Formula I or acrylic based polymers, or derivatives thereof, especially when they are used in topical formulations.

The polymers of Formula I and II have end groups at both ends attached to the oxygen atoms in the polymer of Formula I or the carbon atoms of Formula II. They are hydrogen at both ends.

The compounds of the present invention include polymers having repeating anionic units of Formula I and Formula II, and wherein at least one of R¹, R², R³ and R⁴ in the cellulose based polymers and R⁵ in the anonic acrylic based polymer are substituted with chemical moieties containing one or more carboxylic acids, sulfuric acids, sulfonic acids, acid anhydride, carboxylates, sulfates, sulfonates, or combinations thereof. As defined hereinbelow, the pKa of at least one of the groups used to directly link to the polymer backbone, is less than about 5.0, and more preferably ranges from 1.0 to about 5.0. If the moiety contains more than one functionality linked to the polymer backbone as defined hereinabove, which is carboxylic acid, sulfuric acid, sulfonic acid, or anhydride, carboxylate, sulfate or sulfonate, the first pKa is preferably less than 5.0, and more preferably less than 4.5. Without wishing to be bound, it is believed that as long as one of the functionality on each of the repeating units, such as carboxylic acid, sulfuric acid, sulfonic acid, anhydrides carboxylate, sulfate or sulfonate has a pKa of less than about 4.5, the polymer of the present invention is soluble, and mostly dissociated in the aqueous solvent, such as the vaginal lumen, and thus can be used to treat STDs. The degree of substitution (homogeneous or heterogeneous) per repeat unit of the polymers, copolymers, or oligomers is such that the resulting molecule is molecularly dispersed and mostly dissociated at the pH ranging from about 3 to about 14 and more preferably from about 3 to about 5. It is particularly preferred that the polymers, copolymers, and oligomers of the present invention are molecularly dispersed and mostly dissociated at a pH equivalent to that of the vaginal lumen. With respect to HPMCT, the acidic substitutions, such as trimellityl, hydroxypropoxyl, and methoxyl, are such that the compound is soluble in water or aqueous solvent at a pH of 4.0.

It is preferred that the pKa of the compounds of the present invention is sufficiently low so that one or more free acid groups in these molecules are dissociated at pH values of about 3 or less (i.e., at a pH of about 3 to about 14). The dissociated acidic groups of the invention are important for both the solubility and biologic activity of the molecule. For example the pH in the vaginal lumen is in the range of 3.4 to 6.0 (S. Voeller, D. J. Anderson, “Heterosexual Transmission of HIV.” JAMA 267, 1917-1918 (2000)), and may undergo a transient increase in pH upon the addition of semen which has a pH of about 8.0. Therefore, the polymers of the present invention remain in its molecularly dispersed state in solution and maintains its biological activity in the entire pH range that would be encountered under these physiologic conditions (i.e., pH ranging from about 3 to about 14 and more preferably pH ranging from 3 to 10). In addition, the molecule remains in a dissociated state in order to be capable of interacting via electrostatic forces, especially within the vaginal pH range. For example, the pKa's of the acid functionality on CAP having one trimellityl per glucose unit is about 4.60, 2.52, and 3.84. The remaining free carboxylic acid group in CAP has a pKa of about 5.3 and thus it will not be dissociated in the pH of the vaginal environment.

Polymers, copolymers or oligomers having carboxyl groups that are not dissociated have very low solubility in water at low pH; as the pH is raised, equilibrium shifts to the formation of the ionized form with increasing water solubility. Thus, the pH at which cellulosic polymers become soluble can be controlled by adjusting both the kind of carboxylic acid moiety linked to the polymer or oligomer backbone, and the degree of substitution. The present invention involves the use of carboxylic acid substituted oligomers or polymers which retain their solubility at pH of about 3 or less (that is they remain molecularly dispersed and mostly dissociated in solution) to retard or prevent the transmission of infectious diseases and to prevent, retard, or treat sexually transmitted diseases. In addition these oligomers or polymers can be used in combination therapies to treat STDs and other infectious organisms, as additives or as an adjuvant to other therapeutic formulations, as a plasticizer, as part of a cosmetic formulation, as a disinfectant for general household or industrial use, as an active agent to reduce bacterial, viral or fungal contamination in ophthalmic applications such as eye drops or contact lens solutions, and in toothpaste or mouthwash formulations.

In one embodiment of the present invention, anionic cellulose based polymers of compounds described in this application, such as HPMCT, HPMCP, CAT, and CAP, are further derivitized by the addition of a sulfate or sulfonate or other strong acid group to a free hydroxyl on the polymer for the purpose of increasing the solubility (molecularly dispersed in solution) and dissociation of the functional group over a wide range of pH from about 3 to about 14. These modifications will increase the overall biological effectiveness of the agent under physiologic conditions encountered in the vaginal lumen.

In a preferred embodiment, the hydrophobicity of the compounds of the present invention is tailored simultaneously with the solubility and dissociation properties thereof, by both selecting the intermediate chemical structure and the level of its substitution in the polymer backbone. In the case of the compounds having a cellulosic-based backbone, the anhydride, acid chloride, or other reactive intermediate used to derivatize the polymers will include one or more aromatic (or heterocyclic) rings such that the resulting product possesses the right balance of solubility, hydrophobicity, and level of dissociable functional groups covering the pH range from about 3 to about 14, a condition necessary for desired biological activity in the acidic environment of the vaginal lumen with regard to retarding infectivity as elaborated in this invention. It has been demonstrated by the present invention that a balance between solubility, dissociation and hydrophobicity in the case of HPMCT is in the range of about 0.25 to about 0.7 moles of trimellityl substituent per mole of glucose unit. That is to say an HPMC chain of 100 moles of glucose units in length will have optimally 25 to 70 moles of trimellityl substituents. Equivalent molecules can be tailored to exhibit the balance of properties in HPMCT.

Striking the balance between the ability to remain in the dissociated state over a wide range of pH is important since it is likely that electrostatic and hydrophobic interactions in the resulting polymer (copolymer or oligomer) are both important to molecular binding of said molecule with glycoproteins on viral and cellular surfaces. Without wishing to be bound, it is preferred that interaction with viral or cellular surface proteins may require both electrostatic and hydrophobic forces to affect tight binding. Therefore, the presence of phenyl groups as in the case of trimellitic modifications is desirable for tailoring the hydrophobicity function of the molecule in order to enhance the desired biological activity. According to the present invention, hydrophobicity can be imparted by selecting one of the acidic functionalities described hereinabove, such as carboxylic acid, sulfuric acid, sulfonic acid, or anhydride, with a strong hydrophobic groups such as those bearing one or more aromatic rings including phenyl, naphthyl, and the like with know hydrophobic character, as shown herein. Thus the polymers of the present invention are tailored with a smaller number of strong hydrophobic groups like naphthyl or a larger number of less hydrophobic groups like phenyl. One skilled in the art possesses the ability to strike the above balance between hydrophobicity, solubility and dissociation properties by manipulating the parameters of the modification and degree of substitution to arrive at the desired performance.

The modifications, according to the present invention, are not limited to reactions with anhydrides but include any substitution of R at any of the hydroxyl groups in the cellulosic backbone. It is thus highly desirable to have modified polymers bearing one or more hydrophobic groups such as phenyl and the like. It has been demonstrated by the present invention that such balance could be made in the case of HPMCT at a range of trimellityl substitution of about 0.25 to about 0.7 per glucose unit. This balance and subsequent biological activity can be duplicated with other modifiers by changing conditions and level of substitution. Therefore, it is understood to one skilled in the art that the scope of the invention is not limited to the discrete formulae or examples in the specification.

For acrylic-based polymers, a similar balance between hydrophobicity, solubility and dissociation is effected to affect the biological function needed to suppress infectivity or STD transmission. For example, in MVE/MA-like polymers, desired functional groups may be incorporated into the polymer either by selectively substituting the R⁵ group of the vinyl co-monomer used, or by mixing under the proper conditions the resulting anhydride with the appropriate R—OH-bearing intermediates as shown in Scheme 1. It is thus feasible using a variety of strategies to incorporate moieties such as those shown in Table 1 into the acrylic-based polymer. For the purpose of the present invention, it is preferable to have a molecularly dispersed polymer that remains dissociated in the pH range from about 3 to about 14, and possesses a level of hydrophobicity that would be optimal for blocking infectivity with STD causing agents. Further, introduction of sulfate or sulfonate groups, or other groups with low pKa values brings favorable solubility and dissociation parameters to very low pH levels (e.g. ≦1.0). One skilled in the art can readily ascertain the suitable reaction conditions to achieve the latter result.

It is yet another embodiment of the present invention to include both strong and weak acid groups in the polymer or copolymer, either cellulosic- or acrylic-based such as those described in the instant specification. Weak acid groups include carboxylic groups having low pKa values as given in Table 1. Strong acid groups include sulfate, sulfonate, or others with low pKa values in the range of 1.0 or below. Resulting molecules possessing the properties given in polymers such as HPMCT or acrylic equivalents and including strong acid groups such as sulfate and sulfonates will operate by more than one mechanism to prevent infectivity and transmission of STDs. For example, the presence of sulfate groups in a polymeric molecule is known to strongly bind to the V3 loop of HIV-1 gp 120 (Esté, J. A., Schols, D., De Vreese, K., Cherepanov, P., Witvrouw, M., Pannecouque, C., Debyser, Z., Desmyter, J., Rando, R. F., and De Clercq, E., “Human immunodeficiency virus glycoprotein gp120 as the primary target for the antiviral action of AR177 (Zintevir).” Mol. Pharm. 53:340-345 (1998)), and thus the addition of sulfate or sulfonate groups to the cellulose molecules of Formula I or acrylic molecules of Formula II, such as in a molecule like HPMCT, will expand the spectrum of activity by conferring to the new molecule the ability to act via multiple distinct mechanisms. An example of a sulfate or sulfonated moiety in the cellulose backbone is illustrated by the substitution of, but not limited to, the anhydride of 2-sulfobenzoic acid, as shown in Table 1. The incorporation a sulfate or sulfonated moiety into a cellulose backbone along with carboxylic acid groups is readily apparent to one skilled in the art, e.g., the polymer backbone is substituted by, but not limited to the anhydride of 4-sulfo-1,8-naphthalic acid, as shown in Table 1. Furthermore, the position of the sulfate or sulfonate groups on the ring structures can be varied to adjust performance of the resulting polymer.

In one aspect, of the present invention, R¹, R², R³, and R⁴ in Formula I or R⁵ in Formula II is an aliphatic or aromatic moiety containing more than one carboxylic acid groups such that once covalently attached to the polymer, copolymer, or oligomer backbone the resultant compound remains molecularly dispersed and mostly dissociated in solution at a range of pH from about 3 to about 14, and more preferably from about pH 3 to about pH 5;

In another aspect, the oligomer or polymer in Formula I is hydroxylpropyl methyl cellulose (HPMC)-based.

In another aspect, the oligomer or polymer in Formula I is cellulose acetate based.

In another aspect, one of R¹, R², R³, and R⁴ in Formula I is derived from the reaction with trimellitic anhydride, and the resultant molecule is hydroxypropyl methylcellulose trimellitate, abbreviated HPMCT, which can remain molecularly dispersed and mostly dissociated in solution at pH ranging from about 3 to about 14.

In another aspect, R¹, R², R³, and R⁴ in Formula I is derived from the reaction with a mixture of maleic anhydride and acetic acid, and the resultant molecule is hydroxypropyl methylcellulose acetate maleate, abbreviated HPMC-AM, which can remain molecularly dispersed and mostly dissociated in solution at pH ranging from about 3 to about 14.

In another aspect R¹, R², R³, and R⁴ in Formula I is derived from the reaction with a mixture of 2-sulfobenzoic acid cyclic anhydride and acetic acid, and the resultant molecule is hydroxypropyl methylcellulose acetate sulfobenzoate, and can remain molecularly dispersed and mostly dissociated in solution at pH ranging from about 3 to about 14.

In another aspect R¹, R², R³, and R⁴ in Formula I is derived from the reaction with a mixture of trimellitic anhydride and acetic acid, and the resultant molecule is cellulose acetate trimellitate, abbreviated CAT, which is molecularly dispersed and mostly dissociated in solution at pH ranging from about 3 to about 14.

In another aspect R¹, R2, R³, and R⁴ in Formula I is derived from reaction with a mixture of 2-sulfobenzoic acid cyclic anhydride and acetic acid, and the resultant molecule is cellulose acetate sulfobenzoate, which is molecularly dispersed and mostly dissociated in solution at pH ranging from about 3 to about 14.

In another aspect, one of R¹, R², R³, and R⁴ in Formula I is derived from the reaction with a mixture of 2-sulfobenzoic acid cyclic anhydride and acetic acid and, a second anhydride such as an anhydride derived from phthalic or trimellitic acid and the resultant compound remains molecularly dispersed and mostly dissociated in solution at pH ranging from about 3 to about 14.

In another aspect, one of R¹, R², R³, and R⁴ in Formula I is —H, —OH, —CH₃, or —CH₂CH(OH)CH₃.

In another aspect, the oligomer or polymer in Formula II is acrylic-based.

In another aspect, the oligomer or polymer in Formula II is a copolymer of methylvinyl ether and maleic anhydride or other acrylic analogue.

In another aspect R¹, R², R³, and R⁴ in Formula I or R⁵ in Formula II is a single carboxylic acid containing moiety as defined hereinabove.

In a preferred aspect R¹, R², R³, and R⁴ in Formula I or R⁵ in Formula II is selected from the multi-carboxylic acid containing moieties some of which are exemplified in Table 1.

It is preferred that R¹, R², R³, and R⁴ in Formula I is a mixture of —H, or —CH₃, or —CH₂CH(OH)CH₃, and a moiety derived from acetic acid, or any monocarboxylic acid, and (in defined proportions) moieties derived from trimellitic acid, or hydroypropyl trimellitic acid, or any di- or tri-, or multi-carboxylic, sulfonic, or sulfate derived acid as shown in (but not limited to) Table 1 such that upon covalent addition to the cellulose or acrylic polymer backbone, the resultant molecule remains molecularly dispersed and mostly dissociated in aqueous solutions in which the pH ranges from about 3 to about 14 and more preferably from about 3 to about 5.

In an embodiment at least two of R¹, R², R³, and R⁴ are the same. In another embodiment at least three of R¹, R², R³, and R⁴ are the same. In another embodiment R¹, R², R³, and R⁴ are all the same.

It is preferred that in Formula II, R⁶ is H, CH₃ or CH₃CH(OH)CH₃ and R⁵ is a moiety derived from acetic acid, or any monocarboxylic acid, and (in defined proportions) moieties derived from trimellitic acid, or hydroypropyl trimellitic acid, or any di- or tri-, or multi-carboxylic, sulfonic, or sulfate derived acid as shown in (but not limited to) Table 1 such that upon covalent addition to the cellulose or acrylic polymer backbone, the resultant molecule remains molecularly dispersed and mostly dissociated in aqueous solutions in which the pH ranges from about 3 to about 14 and more preferably from about 3 to about 5.

The present invention provides methods for the treatment or prevention, or prevention of transmission of a viral, bacterial, or fungal infection in (or to) a host, which comprises administering to the host a therapeutically effective amount of an anionic cellulose or acrylic based polymer, a prodrug of either or a pharmaceutically acceptable salt of said anionic cellulose based polymer or acrylic based polymer or prodrug of either.

The present invention provides such methods wherein the viral infection is caused by viruses such as herpes virus, retrovirus, papillomavirus, and the like. The anionic cellulose based polymers and the acrylic based polymers of the present invention are preferably used to treat or prevent viral infections caused by such viruses as HIV-1, HIV-2, HPV, HSV1, HSV2, RSV (respiratory syncytial virus), VZV, influenza virus, including both type A, e.g., H5N1 and type B, rhinovirus, SARS (severe acute respiratory syndrome) causing virus, Small Pox virus, Cow pox, Vaccinia virus, heamorraghic fever causing virus, such as the Filoviruses Marburg and Ebola, the Arena viruses such as Lassa Fever Virus and New World Arenaviridae, the Bunyaviruses such as Crimean-Congo hemorrhagic virus, Hanta viruses, Punto Toro and Rift Valley Fever viruses, and the Flaviruses such as Hepatitis C virus, Dengue and Yellow Fever Viruses, and the like.

The present invention also provides such methods wherein the bacterial infection is caused by bacteria including Trichomonas vaginalis, Neisseris gonorrhea Haemopholus ducreyl, Chlamydia trachomatis, Gardnerella vaginalis, Mycoplasma hominis, Mycoplasma capricolum, Mobiluncus curtisii, Prevotella corporis, Calymmatobacterium granulomatis, and Treponema pallidum, and the like.

In addition, the present invention provides such methods wherein the fungal infection is caused by fungi including Candida albicans and the like.

It is preferred that the anionic cellulose- or acrylic-based polymer, a prodrug thereof, or a pharmaceutically acceptable salt of said anionic cellulose based polymer or prodrug is molecularly dispersed and mostly dissociated in an aqueous solution at pH ranging from about 3 to about 14.

In one embodiment of the present invention, said viral infection is caused by a retrovirus.

In one preferred embodiment the present invention, said anionic cellulose-based polymers are compounds of Formula I.

In one preferred embodiment the present invention, said anionic acrylic-based polymers are compounds of Formula II.

In another preferred embodiment of the present invention, said anionic cellulose based polymers are hydroxylpropyl methyl cellulose (HPMC)-based polymers, cellulose acetate (CA)-based polymers, hydroxylpropyl methylcellulose trimellitate (HPMCT)-based polymers, hydroxylpropyl methylcellulose acetate maleate (HPMC-AM)-based polymers, hydroxylpropyl methylcellulose acetate sulfobenzoate-based polymers, cellulose acetate trimellitate-based polymers, and cellulose acetate sulfobenzoate-based polymers.

In another preferred embodiment of the present invention, said anionic acrylic based polymers are methyl vinyl ether and maleic anhydride (MVE/MA) based polymers.

In another embodiment, the viral, bacterial, or fungal infection is caused by microorganisms that can cause infections in ophthalmic, cutaneous, or nasopharyngeal or oral anatomic sites of a host.

In one preferred embodiment, the host is human.

The compounds of the present invention can be prepared by methods well known in the art. The synthesis of anionic cellulose based compounds can be prepared by the methods described by Kokubo et al. (Kokubo H., Obara, S., Imamura, K., and Tanaka, T., “Development of Cellulose Derivatives as Novel Enteric Coating Agents Soluble at pH 3.5 to 4.5 and Higher.” Chem. Pharm. Bull 45:1350-1353 (1997)) and as described in U.S. Pat. Nos. 6,165,493; 6,462,030; 6,258,799; and Japanese Patent JP-A 8-301790, the contents of all of which are incorporated by reference. Anionic acrylic copolymers such as MVE/MA and other acrylic based materials can be prepared from starting materials such as methyl vinyl ether and maleic anhydride. Multiple different routes for preparing compounds of Formulae I and II are available. Typically those compounds can be prepared via the formation of an ester or ether linkage using anhydride and alcohol containing intermediates. One skilled in the art of organic or polymer chemistry would ascertain the conditions to make those compounds without any undue experimentation.

Scheme 1 below illustrates one route of the synthesis of acrylic copolymers consisting of poly methyl vinyl ether and maleic anhydride (MVE/MA). The synthesis of MVE/MA involves the slow addition of molten maleic anhydride and methyl vinyl ether at 58° C. over a two hour period. The reaction is performed under pressure (e.g. 65 phi). The anhydride ring can be opened up to yield the corresponding half esters using an appropriate alcohol intermediate. Alternatively the dicarboxylic acid can be achieved by the addition of H₂O. In addition the mono or mixed salt variants can be easily prepared. R⁶ in Formula II for MVE/MA is methyl in the scheme below, but this is for illustrative purposes the reaction scheme can be performed with the other definitions of R⁶.

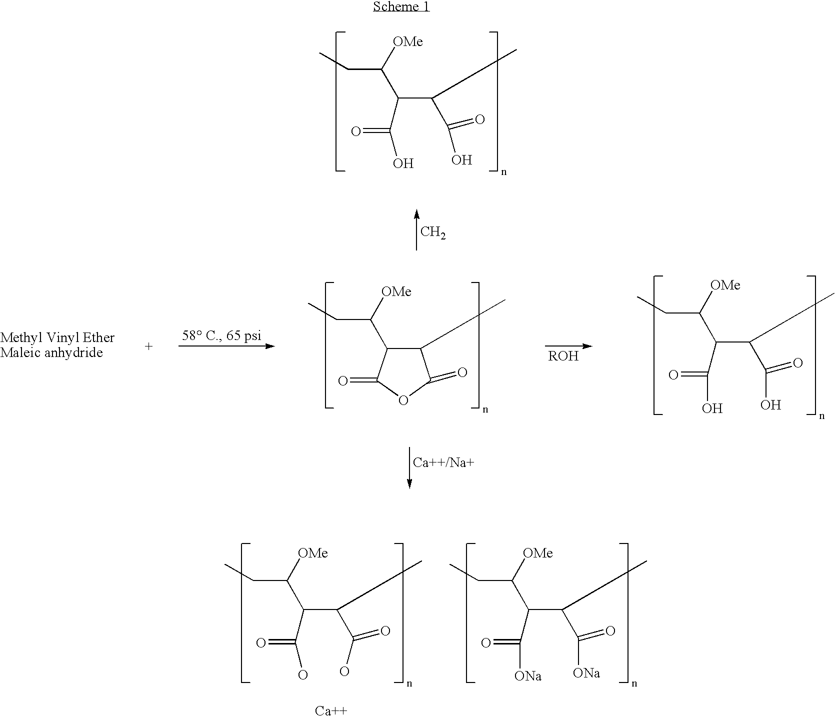

The therapeutic and/or prophylactic effective amount of a compound of Formula I or II of the present invention varies with the particular compound selected, but also with the route of administration, the nature of the condition for which treatment is required, and the age and condition of the patient. It would be appreciated by one skilled in the art that the therapeutic and prophylactic effective amounts of a compound of Formula I or II of the present invention are both easily determined by one of ordinary skill in the art. Of course, it is ultimately at the discretion of the attendant physician or veterinarian. Preferably, however, a suitable dose, regardless of being used for the treatment or prophylaxis of bacterial, fungal, or viral infections, ranges from about 0.01 to about 750 mg/kg of body weight per day, more preferably in the range of about 0.5 to about 60 mg/kg/day, and most preferably in the range of about 1 to about 20 mg/kg/day for systemic administration, or for topical applications, a preferable dose ranges from about 0.001 to about 25% wt/vol, more preferably in the range of about 0.001 to about 5% wt/vol of formulated material. Alternatively the polymer of the present invention, can be micro-dispersed (micronized) instead of molecularly dispersed in solution. If thus applied, under these circumstances, the preferred effective amount of the dose ranges from about 0.01 to about 25 weight percent of micronized cellulosic- or acrylic-based polymer or oligomer derivative.

The desired dose according to one embodiment is conveniently presented in a single dose or as a divided dose administered at appropriate intervals, for example as two, three, four or more doses per day.

While it is possible that for use in therapy or prophylaxis, a compound of Formula I or II of the present invention is administered as a single agent molecularly dispersed in an aqueous solution, it is preferable according to one embodiment of the invention, to present the active ingredient as a pharmaceutical formulation. The embodiment of the invention thus further provides a pharmaceutical formulation comprising a compound of Formula I or II or a pharmaceutically acceptable salt thereof together with one or more pharmaceutically acceptable carriers, diluents or vehicles thereof and, optionally, other therapeutic and/or prophylactic ingredients. The carrier(s) must be “acceptable” in the sense of being compatible with the other ingredients of the formulation and not deleterious to the recipient thereof.

According to one embodiment of the present invention, pharmaceutical formulations include but are not limited to those suitable for oral, rectal, nasal, topical, (including buccal and sub-lingual), transdermal, vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods according to this embodiment include the steps of bringing into association the active compound with liquid carriers or finely divided solid carriers or both and then, if necessary, shaping the product into the desired formulation.

According to another embodiment, pharmaceutical formulations suitable for oral administration are conveniently presented as discrete units such as capsules, cachets or tablets, each containing a predetermined amount of the active ingredient, as a powder or granules. In another embodiment, the formulation is presented as a solution, a suspension or as an emulsion. In still another embodiment, the active ingredient is presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds in Formula I or II according to an embodiment of the present invention are formulated for parenteral administration (e.g. by bolus injection or continuous infusion) and may be presented in unit dose form in ampoules, pre-filled syringes, small volume infusion or in multi-dose containers with an added preservative. The compositions may take such forms as suspensions, solutions, emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

For topical administration to the epidermis (mucosal or cutaneous surfaces), the compounds of Formula I or II, according to one embodiment of the present invention, are formulated as ointments, creams or lotions, or as a transdermal patch. Such transdermal patches may contain penetration enhancers such as linalool, carvacrol, thymol, citral, menthol, and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Pharmaceutical formulations suitable for topical administration in the mouth include lozenges comprising active ingredient in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

In another embodiment of the present invention, a pharmaceutical formulation suitable for rectal administration consists of the active ingredient and a carrier wherein the carrier is a solid. In another embodiment, they are presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

According to one embodiment, the formulations suitable for vaginal administration are presented as pessaries, tampons, creams, gels, pastes, foams, or sprays containing in addition to the active ingredient such carriers as are known in the art to be appropriate.

According to another embodiment, the formulations suitable for vaginal administration can be delivered in a liquid or solid dosage form and can be incorporated into barrier devices such as condoms, diaphragms, or cervical caps, to help prevent the transmission of STDs.

For intra-nasal administration the compounds, in one embodiment of the invention, are used as a liquid spray or dispersible powder or in the form of drops. Drops may be formulated with an aqueous or non-aqueous base also comprising one or more dispersing agents, solubilizing agents, or suspending agents. Liquid sprays are conveniently delivered from pressurized packs.

For administration by inhalation, the compounds of Formula I or II, according to one embodiment of the invention, are conveniently delivered from an insufflator, nebulizer or pressurized pack or other convenient means of delivering an aerosol spray.

In another embodiment, pressurized packs comprise a suitable propellant such as dichlorodifluoromethane, trichlorofluoromethane, dichlorotetrafluoroethane, carbon dioxide or other suitable gas.

In another embodiment, the dosage unit in the pressurized aerosol is determined by providing a valve to deliver a metered amount.

Alternatively, in another embodiment, for administration by inhalation or insufflation, the compounds of Formula I or II, according to the present invention, are in the form of a dry powder composition, for example, a powder mix of the compound and a suitable powder base such as lactose or starch. In another embodiment, the powder composition is presented in unit dosage form in, for example, capsules or cartridges or e.g., gelatin or blister packs from which the powder may be administered with the aid of an inhalator or insufflator.

In one embodiment, the above-described formulations are adapted to give sustained release of the active ingredient.

The present invention also provides methods of using the compounds of Formula I or II or combination thereof alone or in combination with other therapeutic agents, a.k.a. combination therapy. Combination therapy as used herein denotes the use of two or more agents simultaneously, sequentially, or in other defined pattern for the purpose of obtaining a desired therapeutic outcome. A desired therapeutic outcome includes a reduced risk of spread of a viral, bacterial or fungi disease, such as sexually transmitted disease and the like and/or reduced viral, bacterial or fungi infection upon use of the combination therapy. For use in the treatment or prevention of STDs, the present combination therapy includes the administration of one or more therapeutic agent as described herein simultaneously, sequentially, or in other defined patterns. Preferably, the mode of treatment with respect to the combination therapeutic agents is via topical administration. In addition, it is preferred that the combination therapy includes the administration of one or more topical therapeutic agents along with one or more agents that have a differing route of administration (such as via an injection or an oral route of administration). For example, the polymers of Formula I or II or combination thereof are used in combination therapies with each other in therapeutically effective amounts as defined herein. Alternatively, the polymers of Formula I or II or combination thereof are present in therapeutically effective amounts, as defined herein with other classes of antiviral, antibacterial, or antifungal agents. These latter antiviral, antibacterial or antifungal agents may have similar or differing mechanisms of action which include, but are not limited to, anionic or cationic polymers or oligomers, surfactants, protease inhibitors, DNA or RNA polymerase inhibitors (including reverse transcriptase inhibitors), fusion inhibitors, cell wall biosynthesis inhibitors, integrase inhibitors, or virus or bacterial attachment inhibitors.

The compounds of Formula I or II or combination thereof may also be used in combination with other antiviral agents that have already been approved by the appropriate governmental regulatory agencies for sale or are currently in experimental clinical trial protocols.

In one embodiment, the compounds of Formula I or II or combination thereof are employed together with at least one other antiviral agent chosen from a list that includes but is not limited to antiviral protease enzyme inhibitors (PI), virus DNA or RNA or reverse transcriptase (RT) polymerase inhibitors, virus/cell fusion inhibitors, virus integrase enzyme inhibitors, virus/cell binding inhibitors, virus or cell helicase enzyme inhibitors, bacterial cell wall biosynthesis inhibitors or virus or bacterial attachment inhibitors.

In one embodiment, the compounds Formula I or II or combination thereof are employed together with at least one other antiviral agent chosen from amongst agents approved for use in humans by government regulatory agencies.

In one embodiment, the compounds of Formula I or II or combination thereof are employed together with at least one other antiviral agent chosen from amongst approved HIV-1 RT inhibitors (such as but not limited to, Tenofovir, epivir, zidovudine, or stavudine, and the like), HIV-1 protease inhibitors (such as but not limited to saquinavir, ritonavir, nelfinavir, indinavir, amprenavir, lopinavir, atazanavir, tipranavir, or fosamprenavir), HIV-1 fusion inhibitors (such as but not limited to Fuzeon (T20), or PRO-542, or SCH-C), and a new or emerging classes of agents such as the positively charged class of polymers and oligomers know as polybiguanides (PBGs). In addition the polymers of Formula I or II or combination thereof are used in combination with other polyanionic compounds especially those bearing a sulfate or sulfonate group.

In one embodiment, the polymers described herein, alone or in combination are employed together with at least one other antiviral agent chosen from amongst herpes virus DNA polymerase inhibitors (such as acyclovir, ganciclovir, cidofovir, etc.), herpes virus protease inhibitors, herpes virus fusion inhibitors, herpes virus binding inhibitors, and/or ribonucleotide reductase inhibitors.

In one embodiment, the polymers described hereinabove or in combination are employed with at least one other antiviral agent chosen from Interferon-αand Ribavirin, or in combination with Ribavirin and Interferon-α.

In a further embodiment, the polymers of Formula I or II or combination thereof are employed together with at least one other anti-infective agent known to be effective against various pathogenic organisms such as, but not limited to, Trichomonas vaginalis, Neisseris gonorrhoeae Haemopholus ducreyi, or Chlamydia trachomatis, Gardnerella vaginalis, Mycoplasma hominis, Mycoplasma capricolum, Mobiluncus curtisii and Prevotella corporis, Calymmatobacterium granulomatis, Treponema pallidum, and Candida albicans.

The combinations referred to above are conveniently presented for use in the form of a pharmaceutical formulation. Thus, the pharmaceutical formulations comprising a combination as defined above together with a pharmaceutically acceptable carrier, vehicle or diluent therefor comprise a further aspect of the invention.

The individual compounds of such combinations may be administered either sequentially or simultaneously in separate or combined pharmaceutical formulations.

When the compound of Formula I or II, or a pharmaceutically acceptable salt or formulation thereof is used in combination with a second therapeutic agent active against the same or different virus, the same or different strain of bacteria, or the same or different type of fungal infection, the dose of each compound may either be the same as or differ from that when the compound is used alone. Appropriate doses will be readily determined by those skilled in the art, or by the attending physician.

Further, compounds of Formula I and Formula II and the pharmaceutically acceptable formulations thereof can be vehicles or adjuvants for use in therapeutic and cosmetic applications, a thickener for topical administration or as an anti-infective agent.

The following examples are provided to illustrate various embodiments of the present invention and shall not be considered as limiting the scope of the present invention in any way. Furthermore, they illustrate different synthetic means for preparing compounds of the present invention. These synthetic procedures are representative and illustrative of the procedures for preparing the compounds of the present invention.

### EXAMPLES

Cellulose acetate phthalate (CAP), cellulose acetate trimellitate (CAT), and hydroxypropyl methyl cellulose phthalate (HPMCP) and commercially available. They were purchased from Sigma/Aldrich, and these three polymers had carboxylic acid moiety substitution patterns between 32 and 35 weight percent, and average molecular weight distributions in the range of 50 kD. The Dextran sulfate (DS) used had an average molecular weight of about 500 KD. All polyanions used in these studies were suspended in 50 nM sodium citrate buffer pH 7.0 at concentrations ranging from 2% to 5% and were stored at 4° C. until use.

### Example 1

Synthesis of Acrylic Based Polymers, Copolymers or Oligomers.

Acrylic based polymers and copolymers are obtained using a variety of techniques that are apparent to one skilled in the art. For example, a synthetic scheme to synthesize MVE/MA involves the addition of 404.4 parts cyclohexane, and 269.6 parts ethyl acetate into a 1 liter pressure reactor. Next 0.3 parts of t-butylperoxypivilate are added at 58° C. in three installments of 0.1 part each at times 0, 60 and 120 minutes from the first addition. Seventy-five parts of molten maleic anhydride and 49.0 parts of methyl vinyl ether are mixed together and gradually added to the reaction vessel at 58° C. and 65 psi over a 2 hour period of time. The reaction mixture is then held at 58° C. for two hours after the last addition of initiator. (The presence of maleic anhydride is determined by testing with triphenyl phosphene to ascertain the extent of the completion of the reaction; the resulting complex precipitates out of solution). After the reaction is complete, the product is cooled to room temperature, filtered and dried in a vacuum oven. If cross-linked copolymer is desired, then 6 parts of 1,7 octadiene is added to the reaction vessel before the addition of the t-butylperoxypivilate.

### Example 2

Derivatization of Acrylic-Based Polymers, Copolymers or Oligomers to Achieve Enhanced Solubility at Low pH.

One skilled in the art could imagine several different mechanisms for creating diversity within the acrylic polymer or copolymer motif that will allow for variation in charge density or hydrophobicity. One mechanism is to interchange maleic anhydride in Example 1 above with any anhydride derivative of moieties containing one or more carboxylic acid group as shown in, but not limited to, Table 1. Alternatively a mixture of two or more anhydride containing moieties, derived from examples shown in Table 1, can be used to generate a polymer with alternating charged moieties. These moieties could be aliphatic or aromatic.

A second mechanism to modify the hydrophobicity or electrostatic charge of an acrylic based polymer is to replace methyl vinyl ether described in Example 1 above with styrene, methyl methacrylate phthalic acid, trimellitic acid, vinyl acetate, or N-butyl acrylate. In addition, polymers or copolymers that incorporate coumarone, indene and carbazole can also be prepared. These aromatic structures, linked as copolymers to moieties bearing carboxylic acid, sulfonates or sulfates add variation to the hydrophobicity and electrostatic profile of the polymer or copolymer and are readily synthesized using standard technology (See. e.g. Brydson, J. A. Plastics Materials, second edition, Van Nostrand Reinhold Company, New York (1970)).

A third mechanism one could employ to alter the hydrophobic or electrostatic nature of a copolymer as depicted in Formula II, and Scheme 1 is to modify the anhydride intermediate of the copolymer to form a half ester. To do this, the anhydride ring is opened up in the presence of the alcohol intermediate of the desired moiety to be added as shown in Scheme 1. Some examples of compounds with desirable functional groups for addition to the polymer backbone are shown in Table 1.

### Example 3

Synthesis of Cellulose-Based Polymers and Copolymers or Oligomers.

For the synthesis of hydroxypropyl methylcellulose trimellitate (HPMCT), 700 grams of HMPC is dissolved in 2100 grams of acetic acid (reagent grade) in a 5 liter kneader at 70° C. Trimellitic anhydride (Wako Pure Chemical Industries) and 275 grams of sodium acetate (reagent grade) as a catalyst are added and the reaction is allowed to proceed at 85 to 90° C. for 5 hours. After the reactions, 1200 grams of purified water is poured into the reaction mixture, and the resultant mixture is poured into an excess amount of purified water to precipitate the polymer. The crude polymer is washed well with water and then dried to yield HPMCT. Hydroxypropyl methylcellulose acetate maleate (HPMC-AM) is synthesized similarly using a mixture of acetic and maleic anhydride in place of trimellitic anhydride. Other methods can be employed to generate the carboxylic acids substituted polymers of the present invention.

The degree of carboxylic acid substitution is dependent upon the conditions used and the purity of the reactants. For example, Kokubo et al. (“Development of cellulose derivatives as novel enteric coating agents soluble at pH 3.5-4.5 and higher.” Chem. Pharm. Bull. 45:1350-1353 (1997)) demonstrate how the degree of substitution per unit of glucose of methoxyl, hydroxypropoxyl, and trimellityl can have large differences in the pH solubility of the resulting HPMCT polymer. Therefore, given the prior art, it was not obvious that simply changing the substitution from a dicarboxylic acid moiety like phthalate to a tricarboxylic acid moiety like trimellitate would yield a compound with superior solubility and carboxylilc acid group dissociation at low pH and at the same time be an effective agent against multiple infectious organisms. Just as each compound and each variant with respect to substitution per mole of glucose, needed to be tested empirically for their solubility and carboxylic acid dissociation profiles, there also was no a priori predictive indicator of how each would affect the different infectious agents described in this application.

The degree of substitution of the HPMCT polymer used in the following assay contained approximately 35 mole percent trimellitate, that is 0.35 moles of trimellityl per mole of b1-4 linked glucose dimer (one repeat unit). The effectiveness of HPMCT at 35% trimellitate substitution presented in this application is representative of the effectiveness of the compounds of the present invention an as anti-viral agent. Other HPMCTs having variations in the mole percent substitution can also be synthesized. It is to be noted that in the following examples unless indicated to the contrary, the HPMCT utilized has 35% trimellitate substitution per mole of repeat unit.

In addition to the electrostatic enhancement provided by the trimellitate group to the cellulose backbone, the ability of the polymer to interact with viral glycoproteins is also enhanced by the presence of the substituents described herein, e.g., phenyl ring. Specific hydrophobic forces can help stabilize the interaction of the polymers, copolymers and oligomers of this invention with HIV-1 gp120 and gp41. Therefore, without wishing to be bound, it is believed that the polymers of Formula I and II are effective in that they strike a balance between electrostatic and hydrophobic interaction capability so to enhance molecular binding of said compounds with target glycoproteins on viral and/or cellular surfaces. It is believed, without wishing to be bound, that interaction with HIV-1 viral surface proteins including gp120 and gp41 specifically requires both electrostatic and hydrophobic interaction to effect tight binding that would prevent viral interaction with cell surface receptors such as CD4 or co-receptors like CCR5 and CXCR4. In order to achieve tight binding that blocks infectivity of cells, the polymer is preferably present in the molecularly dispersed state. Therefore, the presence of the substituents described hereinabove, such as phenyl groups as in the case of trimellitic modification is desirable for tailoring the hydrophobicity function of the molecule in order to affect the desired biological activity. According to the present invention, hydrophobicity can be imparted by e.g., selecting an intermediate anhydride, or other equivalent modifying reagent, with a strong hydrophobic group such as those bearing one or more aromatic rings including phenyl, naphthyl, and the like with known hydrophobic character. It is thus feasible to tailor the molecule with a smaller number of strong hydrophobic groups, like naphthyl, or a larger number of less hydrophobic groups like phenyl. One skilled in the art possesses the ability to strike the above balance between hydrophobicity, solubility and dissociation properties by manipulating the parameters of the modification and degree of substitution to arrive at the desired performance. The modifications according to the present invention are not limited to reactions with anhydrides but include any substitution at R¹, R², R³ and R⁴ in Formula I and R⁵ in Formula II or any hydroxyl group in the cellulosic backbone skeleton. Therefore the scope of the invention should not be limited by the discrete formulae or examples covered in the specification.

To illustrate the versatility of this application Table 1 lists a representative set of moieties that are covalently linked to a cellulose or acrylic polymer backbone, using the above described procedures, or a procedure similar to it, that someone skilled in the art could realize.

TABLE 1
Substitutions for cellulose or acrylic based oligomers, copolymers, or
polymers.

| | **pKa |
|---|---|
| *R | Values |
| 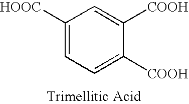 | 2.52, 3.84, 5.2 |
| 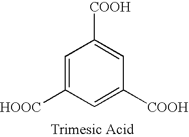 | 3.12, 3.89, 4.7 |
| 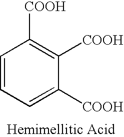 | 2.8, 4.2, 5.87 |
| 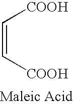 | 1.93, 6.58 |
| 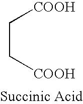 | 4.19, 5.48 |
| 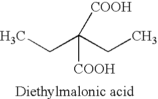 | — |
| 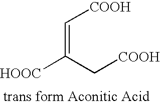 | — |
| MVE/MA copolymer of | 3.51, 6.41 |
| methyl vinyl ether and | |
| maleic acid | |
| 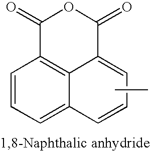 | — |
| 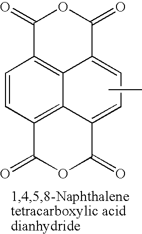 | — |
| 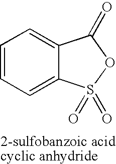 | — |
| 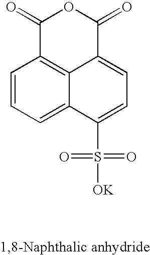 | — |
| 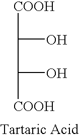 | |
| (+)-2.99, 4.4 (−)-3.03, 4.4 Meso- 3.22, 4.85 | |
| 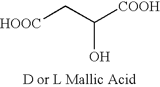 | 3.4, 5.2 |
| Vinyl acetic acid | 4.42 |

*R = the moiety, that when covalently attached to the polymer, copolymer, or oligomer backbone, results in a molecule that is able to remain molecularly dispersed, and mostly dissociated, in solution over a wide range of pH. R as defined, refers to any one of R¹, R², R³, R⁴, or R⁵, as defined herein.
**pKa values given at room temperature and taken from a variety of sources including (Hall, H.K., J. Am Chem. Soc. 79:5439-5441, 1957; Handbook of Chemistry and Physics (Hodgman, C.D., editor on Chief, Chemical Rubber Publishing Company, Cleveland, OH p. 1636-1637, 1951).

In the examples of Table 1, except for maleic and succinic acid, the linkage to the oxygen atom by R¹, R², R³, R⁴ and R⁵ is via an ester through an acyl group of the carboxylic acid or anhydride. However, with respect to the acrylic polymers, the linkage of the maleic acid and succinic acid by R⁵ is obtained by replacing a hydrogen atom of the CH₂ in succinic acid or a hydrogen atom of CH═CH in maleic acid with a bond to the oxygen atom in the polymer. However, the linkage of the maleic and succinic acid of R¹, R², R³ and R⁴ in the cellulose based polymer to the oxygen atom is through the acyl group.

It is understood to one skilled in synthetic organic chemistry that Table 1 represents only a partial list of suitable substituents, and that many more examples are possible provided that no other reactive functionalities are present which would compete with the primary desired reaction of forming substituted cellulose- or acrylic-based polymers or oligomers. One skilled in the art can prepare one or more active compounds in this class by performing the above synthesis or similar methods using combinatorial synthesis or equivalent schemes by altering the monocarboxylic acid moiety, or the di- or tri-carboxylic acid moiety, or a mixed moiety containing both carboxylic acid groups and sulfate or sulfonate groups, or a moiety containing a sulfate or sulfonate group. Furthermore, additional hydrophobicity can be added using techniques known in the art on those resulting molecules. This can be accomplished in a number of ways including the addition of a naphthalene group such as those shown in Table 1 (naphthalene tetracarboxylic dianhydride or naphthalimide) to the cellulose backbone.

Other substituents for R¹, R², R³, R⁴ of Formula I or R⁵ of Formula II are obtained by using a mixture of the moieties identified or suggested herein or in Table 1. Hydroxypropyl methylcellulose acetate maleate (HPMC-AM) is just such a compound in which a mixture of acetic and maleic anhydride is used to derivatize the hydroxypropyl methyl cellulose backbone, and is illustrative of the compounds of the present invention.

Cellulose acetate trimellitate (CAT) is prepared by reacting the partial acetate ester of cellulose with trimellitic anhydride in the presence of a tertiary organic base such as pyridine. It is to be noted that any anhydride could be substituted for trimellitate to produce the corresponding cellulose acetate derivative. Another method to produce molecules having a mixture of functional groups is by simply using a mixture of different anhydrides during the synthesis procedure. For example, using methods that would produce CAP or CAT, the phthalate or trimellitate anhydride could be mixed with 2-sulfobenzoic acid cyclic anhydride in various ratios, to produce polymers or oligomers that bear both phthalate or trimellitate and 2-sulfobenzoate. The addition of 2-sulfobenzoate with phthalate produces a polymer capable of remaining molecularly dispersed in an aqueous solution, and partially dissociated over a greater range of pH than is noted for CAP.

### Example 4

Cellulose Based Polymers and Copolymers or Oligomers Bearing Sulfate or Sulfonate Groups.

As described in Example 3 above one mechanism that is used to introduce sulfate or sulfonate groups onto a cellulose based backbone is to use a moiety such as 2-sulfobenzoic acid anhydride or 4-sulfo-1,8-naphthalic anhydride. It is noted that the substitution at position R¹, R², R³, R⁴, or R⁵ can be obtained by using a mixture of the moiety bearing the sulfate or sulfonate group and moieties having other functionalities, such as carboxylic acid groups.

Alternatively sulfonation can be achieved by direct chemical linkage to the cellulosic-backbone. For example, under mild conditions adducts of sulfur trioxide (SO₃) such as pyridine-sulfur trioxide in aprotic solvents is added to the cellulosic-based polymer or copolymer or oligomer which is prepared in DMF. After 1 hour at 40° C., the reaction is interrupted by the addition of 1.6 ml of water, and the raw product is precipitated with three volumes of cold ethanol saturated with anhydrous sodium acetate and then collected by centrifugation (See, Maruyama, T., Tioda, T, Imanari, T., Yu, G., Lindhardt, R. J., “Conformational changes and anticoagulant activity of chondroitin sulfate following its O-sulfonation.” Carbohydrate Research 306:35-43, (1998)), the contents of which are incorporated by reference.

### Example 5

Cytotoxicity Analysis of Cellulose and Acrylic Polymers.

All compounds were assessed for cytotoxicity using a standard two hour exposure of HeLa or P4-CCR5 target cells to the drug candidates. P4-CCR5 cells (NIH AIDS Reagent Program) are HeLa cells engineered to express CD4 and CCR5 and were utilized in experiments evaluating anti-viral activity of polymers described herein. These and subsequent assessments of cell viability following exposure to the polymers were conducted using the MTT cell viability assay, in which cell viability is measured spectrophotometrically by conversion of MTT (3-[4,5-dimethylthiazol-2-yl]-2,5-diphenyl tetrazolium bromide) to a purple formazan product (see Pauwels, R., Balzarini, J., Baba, M., Snoeck, R., Schols, D., Herdewijn, P., Desmyter, J., and De Clercq, E. “Rapid and automated tetrazolium-based colorimetric assay for the detection of anti-HIV compounds.” J Virol. Methods 20:309-321, (1988), the contents of which are incorporated by reference). In typical assays, P4-CCR5 cells were exposed to the control compound dextran sulfate (DS) and various cellulose- or acrylic-based polymers for 2 hr at concentrations ranging from 0.00001 % to 2%. Cytotoxicity evaluations between 10 min and 6 hr are usually employed because HIV-1 exposure would be most likely to occur during this time period following application of a topical microbicide.

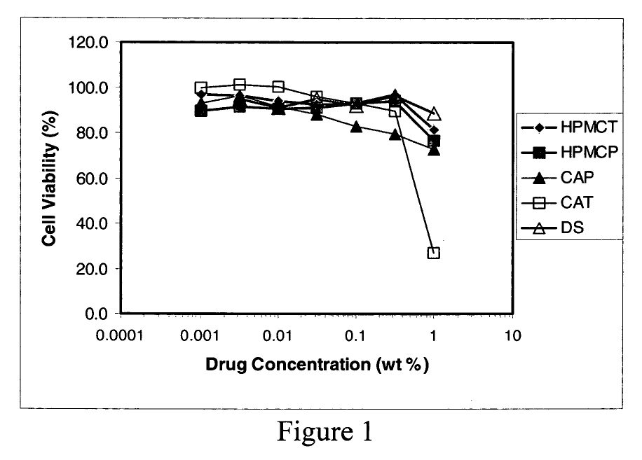

Hydroxypropyl methylcellulose based compounds including, Hydroxypropyl methyl cellulose trimellitate (HPMCT), hydroxypropyl methylcellulose phthalate (HPMCP), and cellulose based compounds such as cellulose acetate phthalate (CAP), and cellulose acetate trimellitate (CAT) were tested in head-to-head fashion for their effect on P4-CCR5 cell metabolism using the MTT assay described above (FIG. 1 and Table 2). The concentration need to inhibit cellular metabolism by 50% (CC50) for each compound tested in this assay system is shown in Table 2.

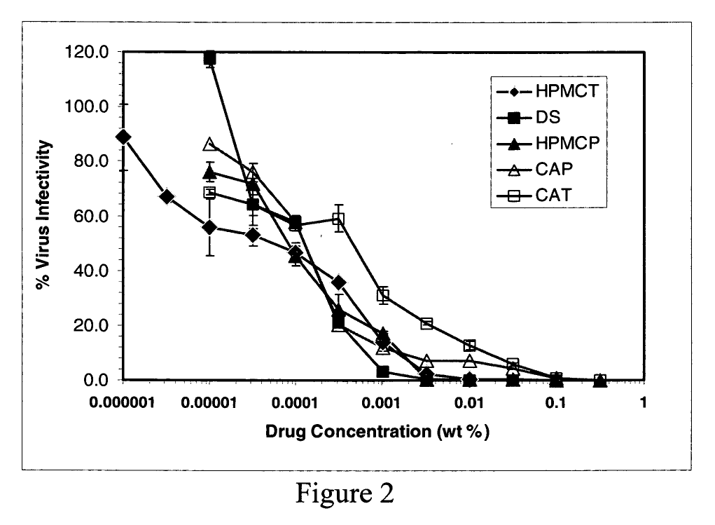

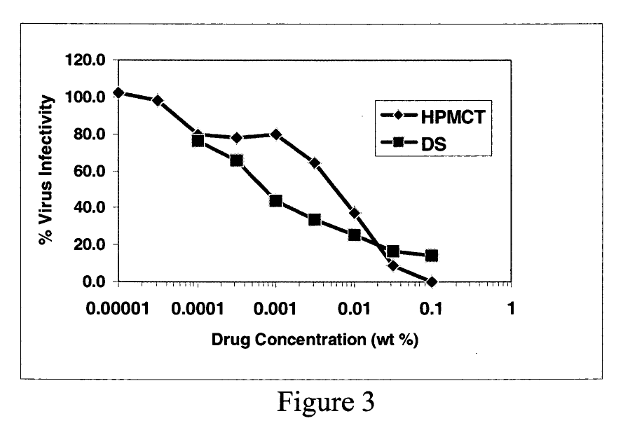

In addition, the toxicity experiments were designed so that the level of exposure and the time of exposure would mimic the efficacy studies in VBI assays shown in FIGS. 2 and 3. In these experiments, P4-CCR5cells were incubated for 2 hrs in the presence of the indicated compounds after which the drug was washed off and the cells further incubated in growth media alone for an additional 48 hrs at 37° C. in a 5% CO₂ atmosphere. At this time the cells were assessed for viability by monitoring their energy production using the tetrazolium dye MTT assay as described by Rando et al. (“Suppression of human immunodeficiency virus type 1 activity in vitro by oligonucleotides which form intramolecular tetrads.” J Biol. Chem. 270:1754-1760 (1995), the contents of which are incorporated by reference). The cytotoxic concentration is many times indicated as the CC50, or concentration of compound needed to reduce cell viability by 50%. This toxicity value, when taken together with the 50% inhibitory concentration (IC50), or concentration needed to reduce cell-free HIV-1IIIB virus infectivity by 50%, is used to tabulate a therapeutic index or TI. The CC50 and IC50 used to plot the TI need to be of a similar format with respect to exposure of virus and/or cells to drug, therefore the exposure time of cells to test compound are the same in the cytotoxicity and VBI assays described below. In FIG. 1 only one compound (CAT) inhibited cell metabolism by greater than 50% at the highest concentration used. Therefore, any TI described in the text is given as a greater than value since the numerator is >1% for all compounds except CAT.

Also presented in Table 2 are the CC50 values obtained when the alternating copolymers of methyl vinyl ether/maleic anhydride (both 216,000 dalton average molecular weight and 1.98 million dalton average molecular weight polymers) and polystyrene/maleic anhydride (120,000 average molecular weight polymer) were assayed for their effect on P4-CCR5 cells.

### Example 6

In Vitro Anti-HIV-1 Efficacy Experiments.

a. Anti-HIV-1 Culture Assays Formats.

In vitro detection of infectivity following exposure of virus cells to cellulose or acrylic polymers relies primarily on the use of indicator cells that produce β-galactosidase (β-gal) as a consequence of HIV-1 infection and a chemiluminescence-based method for quantitating levels of β-gal expression using chemiluminometers, such as the Tropix Northstar™ HTS workstation or TR717™ microplate luminometer. P4-CCR5 MAGI (multinuclear activation of galactosidase indicator) cells are used to detect both X4 and R5 strains of HIV-1 (strains that use the CXCR4 and CCR5 chemokine receptors, respectively). Although this cell line can be treated to visualize β-gal expression in subsequent cell counts, the assays described in this example uses the chemiluminometer to measure β-gal production. The procedure is described at the website http://www.blossombro.com.tw/PDF/Products/Galacto-Star.pdf, the contents of which are incorporated by reference. More specifically, at 48 hr post-infection at 37° C., the cells are washed twice with phosphate buffered saline (PBS) and are lysed using 125 μl of a standard lysis buffer such as 100 mM potassium phosphate (pH 7.8) and 0.2% Triton X-100. HIV-1 infectivity is measured by mixing 2-20 μl of centrifuged lysate with reaction buffer comprised of a Galacton-Star® substrate 50×concentrate (1:50) with Reaction Buffer Diluent comprised of 100 mM sodium phosphate (pH 7.5), 1 mM MgCl₂, and 5% Sapphire-II™ enhancer, incubating the mixture for 1 hr at room temperature, and measuring the subsequent luminescence after assaying for β-galactosidase activity, using the luminometer. This system facilitates the chemiluminescent detection of β-gal in cell lysates. According to the manufacturer, the advantage of this system over cell staining and counting is that it is a fast and easy assay that is highly sensitive and can detect a wide range of β-gal expression. This system, combined with P4-CCR5 MAGI cells, permits sensitive, reproducible detection of infectious virus following exposure to microbicidal compounds 24 to 48 h post-infection.

Viral Binding inhibition (VBI) assays are conducted as follows. On day one, virus (X4-, R5-, or X4R5-tropic; 8 μl at approximately 10⁷ TCID₅₀ per ml) is mixed in RPMI 1640 supplemented with 10% FBS and with test compounds at concentrations decreasing in third log increments from 1%. Aliquots of this mixture are immediately placed on P4-R5 cells and incubated for 2 hr at 37° C. After 2 hr, cells are washed twice with PBS and provided with 2 ml fresh media. After 46 hr at 37° C., the cells are washed twice in PBS and lysed in the well using 125 μl lysis buffer. Activity is assessed as described above.

In cell-free virus inhibition (CFI) assays HPMCT and other cellulose-based polymers are assessed for their ability to inactivate cell-free virus. Assays use a range of concentrations decreasing in third log increments. Briefly, 8×10⁴ P4-CCR5 cells are plated in 12-well plates 24 hr prior to the assay. On the day of the assay, 5 μl of serially diluted compound are mixed with an equal volume of virus (approximately 10⁴-10⁵ tissue culture infectious dose₅₀ (TCID₅₀) per μl) and incubated for 10 minutes at 37° C. After the incubation period, the mixture is diluted (100-fold in RPMI 1640 media including 10% FBS) and aliquots are added to duplicate wells at 450 μl per well. After a 2-hr incubation period at 37° C., an additional 2 ml of new media is added to the cells. At 46 hr post-infection at 37° C., the cells are washed twice with phosphate buffered saline (PBS) and lysed using 125 μl of the lysis buffer described hereinabove. HIV-1 infectivity is measured by mixing 2-20 μl of centrifuged lysate with reaction buffer as described hereinabove, incubating the mixture for 1 hr at RT, and quantitating the subsequent luminescence.

Similar experimental protocols can be utilized for drug candidate treatment of infected cell lines (cell associated virus inhibition (CAI) assays). For example, SupT1 cells (3×10⁶) are infected with HIV-1 IIIB (30 μl of a 1:10 dilution of virus stock) in RPMI media (30 μl) and incubated for 48 hr. Infected SupT1 cells are pelleted and resuspended (8×10⁵ cells/ml). Different concentrations of drug candidates (5 μl) are added to infected SupT1 cells (95 μl) and incubated (10 min at 37° C.). After incubation, the cell and microbicide mixture is diluted in RPMI media (1:10) and 300 μl is added to the appropriate wells in triplicate. In the wells, target P4-CCR5 cells is present. Production of infectious virus results in β-gal induction in the P4-CCR5 targets. Plates are incubated (2 hr at 37° C.), washed (2×) with PBS and then media (2 ml) is added before further incubation (22-46 hr). Cells are then aspirated and washed (2×) and then incubated (10 min at room temperature) with lysis buffer (125 μl). Cell lysates are assayed utilizing the Galacto-Star™ kit (Tropix, Bedford, Mass.).

In continuous exposure experiments, C-8166 cells (4×10⁴ cells/well) are used as the target for HIV-1 infection (CXCR4 or CCR5 tropic virus strains). HIV-1 is added to the cell culture at a multiplicity of infection of 0.01 and the drug candidate is added at the indicated final concentration at the same time. All three are incubated together for five days without washing the cells. Syncytia formation is monitored at day 3 and day 5. If drug alone is added without virus then the same MTT protocol described in Example 5 is used to monitor for cell viability.

In FIG. 2 and Table 2, the dose response curves and IC50 values for DS, HPMCT, HPMCP, CAT and CAP when used to inhibit HIV-1IIIB in the VBI assay are presented. The results from these experiments show that all compounds were effective inhibitors of HIV-1 in this assay system and fairly similar in their overall activity, with the difference between calculated IC50s for the most (HPMCT IC50=0.00009%) and least (CAT IC50=0.0005%) active cellulose based compounds being less then a factor of 10 (see Table 2).

In FIG. 3 and Table 2, the dose response curve and IC50 value showing the effect of HPMCT on HIV-1BaL in the VBI assay is shown. It is interesting to note that the overall activity against HIV-1BaL is approximately 10-fold lower than that observed against the CXCR4 tropic strain of virus for both HPMCT and DS.

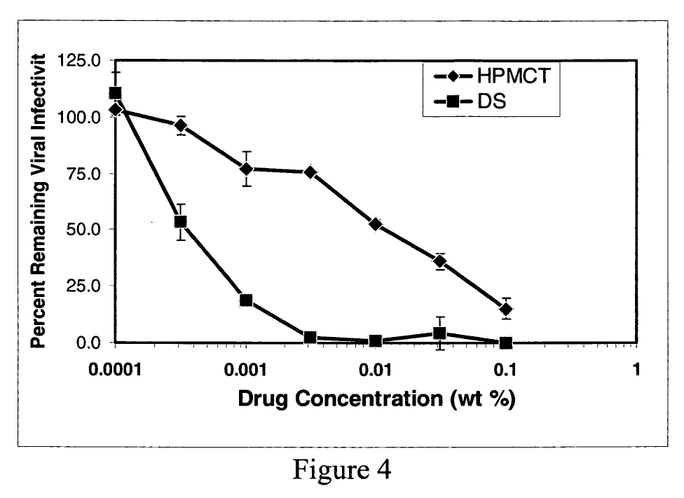

In FIG. 4 and Table 2, the dose response curve and IC50 value with respect to the effect of HPMCT on HIV-1 IIIB in a cell free virus inhibition (CFI) assay are shown. While HPMCT still displays potent activity, it is not as effective in this assay as in the VBI assay, while the control drug DS has a level of activity similar to what it displayed in the VBI assay. Without wishing to be bound, it is believed that the mechanism of action for the molecule of the present invention, as an anti-viral agent, is via interfering with the co-receptor interactions on the cell surface with viral gp120. This activity may occur after gp120 has undergone a conformational change post-binding with the main cellular receptor CD4. Therefore, in this short exposure to HPMCT, the co-receptor binding surface of gp120 may not be accessible to the cellulose polymer. The mechanism of action for DS is known to be via direct interaction with the V3 loop of HIV-1 gp120 (Esté, J. A., Schols, D., De Vreese, K., Cherepanov, P., Witvrouw, M., Pannecouque, C., Debyser, Z., Desmyter, J., Rando, R. F., and De Clercq, E., “Human immunodeficiency virus glycoprotein gp120 as the primary target for the antiviral action of AR177 (Zintevir).” Mol. Pharm. 53:340-345 (1998)). By binding to the V3 loop of the viral glycoprotein, DS interferes with gp120-CD4 interactions. Therefore DS maintains its potency in the short CFI assay duration because it binds to the exposed V3 loop of gp120 and prevents the virus from contacting CD4 in the subsequent steps in the assay. In contrast, HPMCT is believed, without wishing to be bound, to bind to portions of the viral glycoprotein that are generally exposed after the virus binds to the cell (gp120-CD4) and therefore, in the CFI assay system, most of the HPMCT is believed to be diluted out of the system before the virus is exposed to target cells.

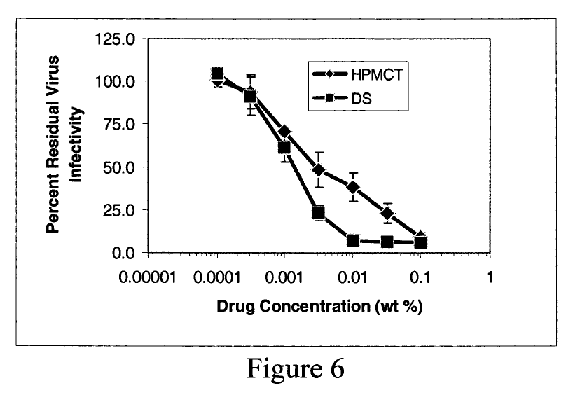

FIG. 6 and Table 2 shows the dose response curve and IC50 value calculated for HPMCT using a cell associated virus inhibition (CAI) assay. In this assay, cell-associated virus was incubated with HPMCT or DS for 10 minutes before dilution and exposure to uninfected reporter cells for 2 hrs. Reporter cells were then washed to remove drug and residual virus in the culture media and further incubated for 48 hrs at 37° C. in a 5% CO₂ atmosphere. The data for this experiment, as depicted in Table 2 and FIG. 6, show that HPMCT is much more effective at inhibiting virus transmission than in the CFI assay. Without wishing to be bound, in this assay, it is possible for CD4 interactions with gp120 to occur before drug is removed from the culture media thereby giving HPMCT access to exposed surfaces of gp120 that form the basis of interaction with the cellular co-receptors CXCR4 or CCR5.

In Table 2 are listed the results obtained using a continuous exposure experiment. In this experiment HPMCT (hydroxypropyl methylcellulose modified with either 35 or 41 mole percent trimellitic acid substitution per mole of sugar, in Formula I) were added to C-8166 cells in the presence of HIV-1 strain IIIB (0.01 multiplicity of infection). Cells, virus and drug candidates were incubated together for five days at which time the cultures were monitored for syncytia formation. In this experiment, the cytotoxicity of each sample was monitored over the same period of exposure to C-1 866 cells and the results are also presented in Table 2.

The alternating acrylic copolymers of either methyl vinyl ether with maleic anhydride (MVE/MA) or polystyrene with maleic anhydride (Polystyrene/MA) were also tested for their effect on HIV-1IIIB in a VBI assay using a two hour exposure of cells to virus in the presence of drug candidate. MVE/MA is commercially available in a variety of different molecular size ranges. In these studies, low molecular weight MVE/MA having an average mol. wt. in the range of 216,000 daltons, and high molecular weight MVE/MA which had an average molecular weight in the range of 1.98×10⁶ (1.98 MM) Daltons were utilized. Polystyrene/MA is also commercially available and the lot used in these studies had an average molecular weight of 120,000 daltons. The alternating copolymers were added to P4-CCR5 cells in tissue culture in the presence of virus (0.01 to 0.1 ml) for 2 hrs. The cells were then washed three times with fresh medium and then further incubated for 48 hr at 37° C. in a 5% CO₂ atmosphere before the level of β-gal production was monitored. The results from this experiment are shown in Table 2. It is clear that MVE/MA itself is not toxic to cells following a 2 hr exposure at concentrations below 0.1%, while its IC50 against HIV-1IIIB in the VBI was determined to be 2.3 μg/ml (low molecule weight MVE/MA), and 2.8 μg/ml for the high molecular weight species which corresponds to 0.00023 and 0.00028 percent respectively. Polystyrene/MA is even less toxic with its CC50 calculated to be >3.0% and its IC50 in the range of 0.0009%.

TABLE 2
Effect of polymers on HIV-1 transmission.

| Assay System | IC50 (wt. %) | CC50 (wt. %)** | TI** |
|---|---|---|---|
| VBI (2 hr exposure) | | | |
| DS | 0.00015 | >1 | >10000 |
| HPMCT | 0.00009 | >1 | >11000 |
| HPMCP | 0.0006 | >1 | >1600 |
| CAP | 0.00015 | >1 | >10000 |
| CAT | 0.00054 | 0.7 | 1296 |
| MVE/MA acrylic | 0.00023 | 0.205 | 891 |
| copolymer 216K mol. wt. | | | |
| fraction | | | |
| MVE/MA acrylic | 0.00028 | 0.19 | 678 |
| copolymer 1.98 MM mol. | | | |
| wt. fraction | | | |
| Polystyrene/MA | 0.0009 | 3.2 | 3555 |
| 120K mol. wt. fraction | | | |
| CFI* (10 min. exposure) | | | |
| DS | 0.0004 | >1 | >2500 |
| HPMCT | 0.01 | >1 | >100 |
| CAI* (10 min. exposure) | | | |
| DS | 0.002 | >1 | >500 |
| HPMCT | 0.003 | >1 | >300 |
| Continuous Exposure | | | |
| Exp. (5 day exposure) | | | |
| HPMCT 35% | 0.000001% | ˜0.1% | >60,000 |
| HPMCT 41% | 0.00000001% | ˜0.1% | >1 MM |

*CFI, and CAI assays used a ten minute incubation of drug with virus before dilution and addition of virus to cells.
**CC50s were calculated using an MTT assay to assess cell viability using either a 48 hrs exposure VBI, CFI, or CAI assays) or a 5 day exposure of cells (continuous exposure assay) to test compound. The therapeutic index (TI) is the cc50/EC50

b. Anti-HIV-1 Efficacy of HPMCT in Combination with the Cationic Polybiguanide PEHMB.

The paradigm for effective HIV-1 therapy (for systemic infections) is the use of combination drug regimens. Combination therapy has proven effective at reducing viremia, delaying the onset of AIDS, and retarding the emergence of drug-resistant virus. At this time the most effective microbicide regimen has not been established in the art. It may be that in order to block sexual transmission of HIV-1 several drugs having different mechanisms of action will need to be applied in the same formulation. Therefore, to augment or broaden the spectrum of HPMCT activity, it was combined with other compounds that have different mechanisms of action against HIV-1. As an example, the following experiments investigated the use of polyethylene hexamethylene biguanide or PEHMB (Catalone, B. J., et al. “Mouse model of cervicovaginal toxicity and inflammation for the preclinical evaluation of topical vaginal microbicides.” Antimicrob. Agents and Chemother. 48:1837-1847 (2004)) combined with HPMCT. PEHMB is a cationic polymer made up of alternating ethylene and hexamethylene units around a biguanide core. In these assays, a 1.0 % wt/vol stock solutions of HPMCT dissolved in 20 mM sodium citrate buffer pH 5.0, and a 5% PEHMB wt/vol solution made up in saline were used as stock solutions.

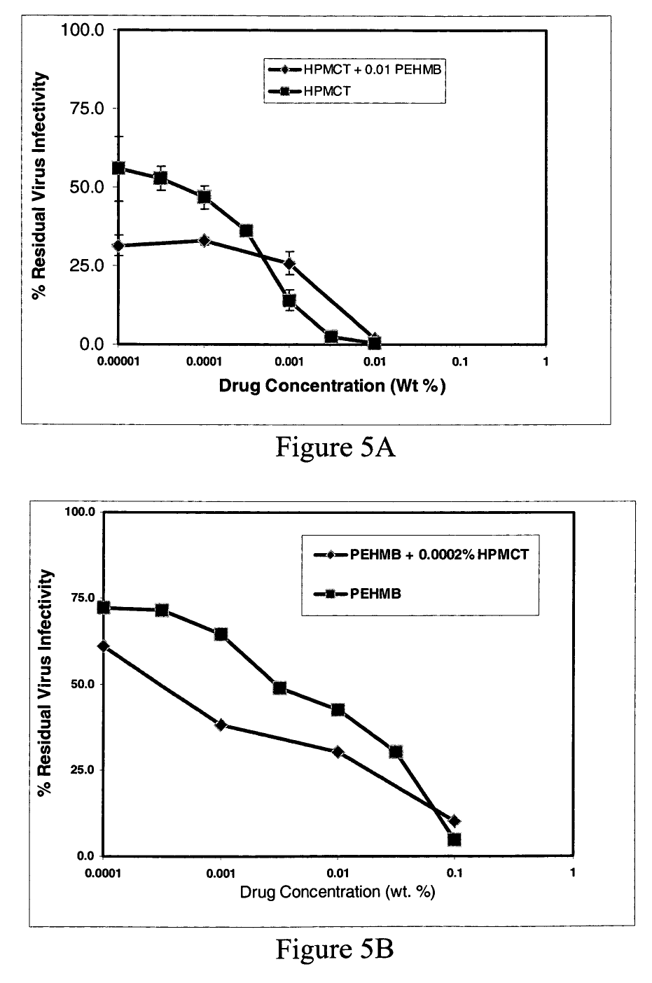

Preliminary combination in vitro cytotoxicity experiments demonstrated that in assays in which the concentration of one component (PEHMB or HPMCT) was varied while the other was kept constant, were non-cytotoxic after a two hour exposure of compounds to test cells, at the concentrations tested. This result was similar to that obtained when PEHMB and HPMCT tested alone (FIG. 1). Using a VBI assay and HIV-1 strain IIIB, HPMCT was equally or more effective when 0.01% PEHMB was combined in the same assay then when using HPMCT alone (FIG. 5A). Similar results were observed when the concentration of HPMCT was held constant at 0.0002% and the concentration of PEHMB was varied (FIG. 5B). These data show that a negatively charged agent can be successfully combined with a positively charged agent.

While logically it appears that negatively-charged polymers like HPMCT would be a poor choice for inclusion in a combination with the positively charged PEHMB, it is believed, without wishing to be bound, that the antiviral activity of PEHMB, and PEHMB-derived molecules, relies not only upon their positive charge, but also upon their three-dimensional shape. Therefore, it may be possible to obtain mixtures of polyanionic compounds with PEHMB at defined ratios which allow for the full expression of the antiviral properties of the individual components without exhibiting any deleterious effects due to their mixing. As seen in FIG. 5, at least within the concentration ranges of PEHMB and HPMCT tested, no antagonistic effects are observed when these two molecules were combined. These data strongly suggest that HPMCT can be used in combination with other agents producing at least additive effects. Furthermore, and it is possible, under the appropriate conditions, to mix low cost polymers with completely different chemical features.

### Example 7

Effect of HPMCT on Herpes Simplex Virus Infections.

Herpes simplex virus plaque reduction assays were performed as described by Fennewald et al. (“Inhibition of Herpes Simplex Virus in culture by oligonucleotides composed entirely of deoxyguanosine and thymidine.” Antiviral Research 26:37-54 (1995), the contents of which are incorporated by reference). This assay is a variation on the cytopathic effect assay described by Ehrlich et al. (Ehrlich, J., Sloan, B. J., Miller, F. A., and Machamer, H. E., “Searching for antiviral materials from microbial fermentations.” Ann N.Y. Acad. Sci 130:5-16 (1965), the contents of which are incorporated by reference). Basically cells such as Vero or CV-1 cells are seeded onto a 96-well culture plate at approximately 1×10⁴ cells/well in 0.1 ml of minimal essential medium with Earle salts supplemented with 10% heat inactivated fetal bovine serum (FBS) and pennstrep (100 U/ml penicillin G, 100 ug/ml streptomycin) and incubated at 37° C. in a 5% CO₂ atmosphere overnight. The medium was then removed, and 50 ul of medium containing 30-50 plaque forming units (PFU) of HSV1 or HSV2, diluted in test medium and various concentrations of test compound are added to the wells. The starting material for this assay was a 0.6% wt/vol stock solutions of HPMCT dissolved in 20 mM sodium citrate buffer pH 5.0. Test medium consists of MEM supplemented with 2% FBS and pennstrep. The virus was allowed to adsorb to the cells, in the presence of test compound, for 60 min at 37° C. The test medium is then removed and the cells are rinsed 3 times with fresh medium. A fmal 100 ul of test medium is added to the cells and the plates are returned to 37° C. Cytopathic effects are scored 40-48 hr post infection when control wells (no drug) showed maximum cytopathic effect.

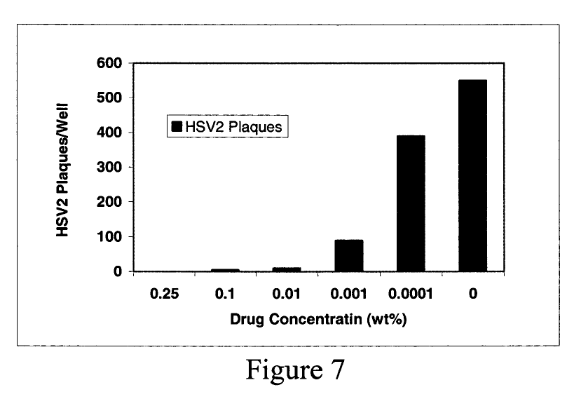

In these experiments HPMCT was added to HSV2 stock for ten minutes before the mixture was applied to cells for 60 min as described above. Forty to 48 hrs post removal of drug from the culture media, the control wells that received no drug treatment had over 500 plaques per well. Wells treated with 0.0001% HPMCT for the indicated amount of time had less than 400 plaques per well, while wells treated with 0.25% HPMCT had no visible plaques, the IC50 for HPMCT in this assay system was below 0.001% (FIG. 7). This result demonstrates the potency of HPMCT as an anti-herpes simplex virus agent.

### Example 8

Effect of HPMCT on Bacterial Pathogens.

To test the effect of HPMCT on bacterial pathogens, the cellulosic-based polymer was dissolved in 20 mM sodium citrate buffer pH 5.0 (0.6% final concentration of stock solution) and then mixed in equal parts with bacterial suspensions as described hereinbelow. First bacteria are sub-cultured 1-2 days prior to the assay by streaking cultures onto suitable agar plates such as Trypticase soy agar. Aseptic technique is used in all aspects of this protocol. A fresh bacterial colony is then used to inoculate 15 ml of 2×culture medium. To the first nine (9) columns of a 96 well plate, 100 μl of the inoculated 2×culture broth is transferred into the wells using a multi channel pipette. The remaining three (3) columns (usually numbered 10-12) are used as a sterility control. To these columns, 100 μl of sterile 2×culture broth is added to each well. The culture medium in the second through eighth rows (usually designated B-H) is diluted by the addition of 80 μl of sterile water to those wells. The volume in wells B through H is at this time 180 μl. The antimicrobial solutions are diluted with water to twice the desired concentration of the uppermost starting concentration. For instance, if the highest test concentration is 1%, the solution is prepared at 2%. For some compounds, no dilution may be needed. To the first row (usually designated as “A”), 100μl of 2×test solution is added to each well. The solution is thoroughly mixed by re-pipetting five times. The total volume of the well is now 200 μl. A 1:10 serial dilution is now performed from Row A through Row G by transferring 20 μl from the higher concentration to the subsequent row using a multi channel pipette. This results in a six log reduction in the concentration of the test compound. In Row G, 20 μl is removed and discarded. No test compound is added to Row H (positive control for growth). The 96 well plate is placed on a shaker in an incubator with the temperature set for the organism of choice (usually 30° C. or 37° C.). After 24 hours, the optical density of the cultures is measured on a 96 well plate reader. Row H serves as a positive control for growth. Columns 10 through 12 serve as negative controls and as a measurement of the optical density of the test solution at different concentrations. Test solution were considered effective at a given concentration if the optical density of the inoculated wells was statistically the same as the negative control wells.

The above described HPMCT formulation was tested for its inactivating effect on the following bacterial pathogens Pseudomonas aeruginosa and Escherichia Coli. Both strains were cultured in Minimal Culture Medium (M9 medium). The results shown in Table 3 indicate that both bacterial strains lost the capacity to replicate after exposure to HPMCT. Vantocil (polyhexamethylene biguanide) is a commercially available disinfectant and was used as a positive control in these experiments. PEHMB is a variant of Vantocil and was also used as a control in these experiments. The activity of HPMCT against the indicated species shows that the compound could be used against a variety of bacterial strains including but not limited to Trichomonas vaginalis, Neisseris gonorrhoeae Haemopholus ducreyi, or Chlamydia trachomatis, Gardnerella vaginalis, Mycoplasma hominis, Mycoplasma capricolum, Mobiluncus curtisii, Prevotella corporis, Calymmatobacterium granulomatis, and Treponema pallidum. Pseudomonas aeruginosa, Streptococcus gordonii, or S. oralis for dental plaque, Actinomyces spp, and Veillonella spp.

TABLE 3
Minimum Inhibitory Concentration for HPMCT against
two bacterial strains.

| | Vantocil* | PEHMB* | HPMCT* |
|---|---|---|---|
| Bacterial strain | MIC (wt. %) | | |
| Escherichia coli | 0.06 | 0.125 | 0.31 |
| Pseudomonas aeruginosa | 0.06 | 0.5 | 0.16 |

*Vantocil is polyhexamethylene biguanide, PEHMB is a variant of Vantocil, and HPCMT is hydroxypropyl methylcellulose trimellitate.

In addition, the acrylic copolymers and HPMCT were tested for their ability to inhibit the growth of Neisseris gonorrhoeae (NG). Compounds were assessed in vitro for bacteriocidal activity against the F62 (serum-sensitive) strain of NG. Briefly, multiple NG colonies from an overnight plate were collected and resuspended in GC media at ˜0.5 OD600. Following 1:10,000 dilution in warm GC media as described by Shell et al. (Shell, D. M., Chiles, L., Judd, R. C., Seal, S., and Rest. R. “The Neisseria Lipooliogosaccharide-specific Alpha-2,3-sialyltransferase is a surface-exposed outer membrane protein”. Infect. Immun. 70:3744-3751 (2002), the contents of which are incorporated by reference), cells (90 μl) were combined with compounds (10 microliters) in 96-well plates to achieve fmal compound concentrations. After incubation in a shaker incubator for 30 to 90 minutes at 37° C., aliquots were removed from each well, diluted 1:10 in media, and spotted on plates in duplicate. Colonies were counted after overnight incubation.

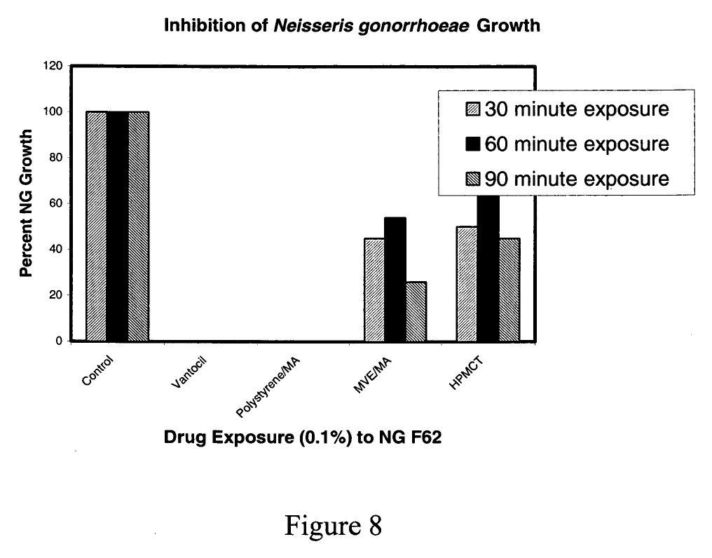

In these assays, a 0.1% solution of the control compound polyhexamethylene bis biguanide (PHMB or Vantocil) and the alternating copolymer of polystyrene with maleic anhydride were able to completely inhibit the growth of NG F62 even with exposure times as short as 30 min (FIG. 8). The acrylic copolymer consisting of methylvinyl ether and maleic anhydride (MVE/MA) was moderately effective at inhibiting NG growth under these conditions with the best inhibition (˜75%, FIG. 8) occurring after a 90 minute exposure of drug to bacteria. HPMCT was less effective, though after a 90 min exposure of drug to NG F62, the inhibition of bacterial growth was significant (˜55%, FIG. 8).

### Example 9

Effect of pH on Solubility of Cellulose Based Polymers.

Kokubo et al. (Kokubo H., Obara, S., Minemura, K., and Tanaka, T., “Development. of Cellulose Derivatives as Novel Enteric Coating Agents Soluble at pH 3.5 to 4.5 and Higher.” Chem Pharm. Bull 45:1350-1353 (1997)) demonstrated that by careful selection of carboxylic acid containing moieties used to link with a cellulosic polymer backbone, the overall pKa of the cellulosic-based polymer could be modified. In addition, in 2000 Neurath reported that CAP and HMPCP are effective agents against sexually transmitted diseases (Neurath A. R. et al. “Methods and compositions for decreasing the frequency of HIV, herpes virus and sexually transmitted bacterial infections.” U.S. Pat. No. 6,165,493. In the Neurath study the investigators appreciated the fact that carboxylic acid groups of CAP and HPMCP are not entirely dissociated at the vaginal pH and actually propose to use micron size particulate formulations of their identified compounds to help get around compound solubility issue (Neurath A. R. et al. U.S. Pat. No. 6,165,493; Manson, K. H. et al. “Effect of a Cellulose Acetate Phthalate Topical Cream on Vaginal Transmission of Simian Immunodeficiency Virus in Rhesus Monkeys,” Antimicrobial Agents and Chemotherapy 44:3199-3202 (2000)). Therefore, the use of chemical moieties to enhance the low pH solubility and significant dissociation of the ionizable functional groups of cellulosic-based, or other polymers and then using those polymers as anti-infective agents are extremely helpful to the overall anti-infective properties of a microbicide. Kokubo et al. (Kokubo H., Obara, S., Minemura, K., and Tanaka, T., “Development of Cellulose Derivatives as Novel Enteric Coating Agents Soluble at pH 3.5 to 4.5 and Higher.” Chem Pharm. Bull 45:1350-1353 (1997)) demonstrate using dissolution time versus pH curves the solubility of compounds such as HPMCT and hydroxypropyl methylcellulose acetate maleate (HPMCAM) in low pH solutions (dissolution pH for these two compounds was determined to be between 3.5 and 4.5) and compared these measured values with historical data on the dissolution pH of CAP (pH 6.2) and HPMCP (pH ˜5.0 to 5.5. These data are consistent with the pKa reported for the second carboxylic acid group on trimellitate (3.84) and phthalate (5.28).

The toxicity and efficacy assays described in Examples 5-7 are routinely performed in eukaryotic cell culture media that is buffered and maintains a pH in the neutral range throughout the time course of the experiment. In those examples, the IC50s and CC50s of the four cellulose-based polymers tested (HPMCT, CAT, HPMCP and CAP) were roughly equivalent. However, to illustrate the point that the trimellitate bearing compounds are differentiated from, and therefore superior to, the phthalate bearing compounds, simple experiments were performed to show that only HPMCT and CAT were able to remain molecularly dispersed and mostly dissociated over the range of pH encountered in the vaginal lumen. This experiment also confirmed the pH dissolution data reported by Kokubo et al. (Kokubo H., Obara, S., Minemura, K., and Tanaka, T., “Development of Cellulose Derivatives as Novel Enteric Coating Agents Soluble at pH 3.5 to 4.5 and Higher.” Chem Pharm. Bull 45:1350-1353 (1997)).

In this experiment, 1% solutions of HPMCT, CAP, CAT and HPMCP (all dissolved in 100 mM Na citrate pH 6.0) were exposed in a drop wise fashion to 0.5N HCl. After each small aliquot of added HCl was added, the samples were vortexed, allowed to settle, observed for clarity and the pH was measured. The results from this mostly qualitative experiment are presented in Table 4. It is readily observed that the solutions containing a trimellitic moiety remained clear at much lower pH values than those containing the phthalate group. In addition, at lower pH, HPMCT and CAT did not ‘gel’ to the same extent indicating that more material remains molecularly dispersed over this range of pH.

TABLE 4
Titration of HCl into 1% solutions of cellulose based polymers.

| | Visual Solution Characteristics at Selected pH | | | | | | | | | |
|---|---|---|---|---|---|---|---|---|---|---|
| Compound | 5.75 | 5.5 | 5.25 | 5.0 | 4.75 | 4.5 | 4.25 | 4.0 | 3.75 | 3.5 |
| CAP | Clear | Clear | Clear | Cloudy | viscous | Thick | — | — | — | — |
| | | | | | cloudy | gelled | | | | |
| | | | | | soln | mass | | | | |
| HPMCP | Clear | Clear | Clear | Cloudy | viscous | viscous | Total | — | — | — |
| | | | | | cloudy | cloudy | gelled | | | |
| | | | | | soln | soln | mass | | | |
| CAT | Clear | Clear | Clear | Clear | Clear | Clear | Viscous | Globular | — | — |
| | | | | | | | cloudy | masses | | |
| | | | | | | | soln | cloudy | | |
| HPMCT | Clear | Clear | Clear | Clear | Clear | Clear | Clear | Viscous | Viscous | Partially |
| | | | | | | | | | cloudy | gelled |

HPCMT is hydroxypropyl methyl cellulose trimellitate,
HPMCP is hydroxypropyl methyl cellulose phthalate,
CAP is cellulose acetate phthalate, and
CAT is cellulose acetate trimellitate.

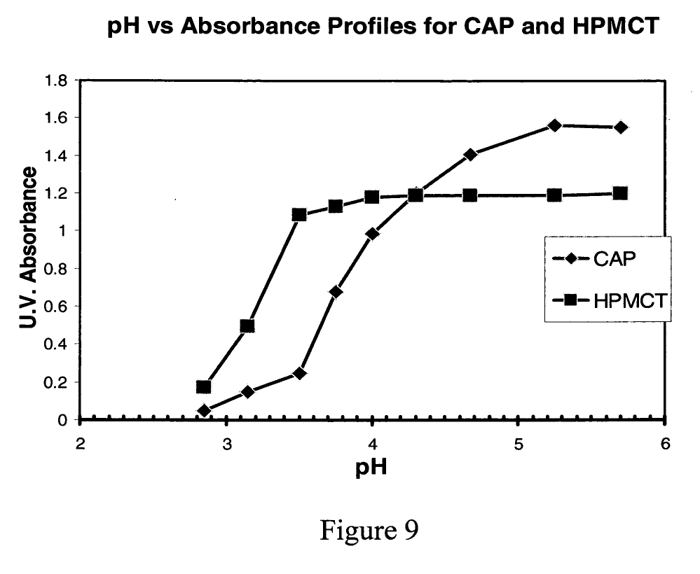

In addition to this experiment in which visual inspection was used to determine the degree of polymer solubility, U.V. absorbance spectroscopy was used to better monitor the effect of pH on the solubility of cellulose-based polymers, CAP and HPMCT. In this experiment (FIG. 9) the degree of HPMCT (0.038% in 1 mM sodium citrate buffer, pH 7) or CAP (0.052% in 1 mM sodium citrate buffer, pH 7) in solution was monitored using U.V. absorbance at either 282 nm (CAP) or 288 nm (HPMCT). The compound samples were slowly made more acidic by the gradual addition of 0.5N HCl. After each addition, the pH was determined and the samples were vortexed for five seconds and then centrifuged using a tabletop centrifuge at 3000 rpm for five minutes. The supernatant was then collected and monitored for the presence of polymer using the absorbance conditions described hereinabove. The results from this experiment show that, as predicted, based on the pKa values of the remaining dissociable carboxylic acid groups of the trimellityl (3.84) and phthalate (5.28) moieties on the cellulose backbone, HPMCT stays in solution at lower pH values than CAP.

### Example 10

Drug Combination Therapy Regimens.

At present, combination therapy comprising at least three anti-HIV drugs has become the standard systemic treatment for HIV infected patients. This treatment paradigm was brought about by necessity in that mono- and even di- drug therapy proved ineffective at slowing the progression of HIV-1 infection to full blown AIDS. Therefore it is also likely that in the development and application of a topical agent to prevent the transmission of STDs, a combination of drugs each having a different or complementary mechanism of action can be envisioned.

The methodology used in the identification of potential combinations for use against HIV-1 has been reported numerous times in the identification and development of anti-HV-1 drugs for systemic applications (Bédard, J., May, S., Stefanac, T., Chan, L., Stamminger, T., Tyms, S., L`Heureux, L., Drach, J., Sidwell, R., and Rando, R. F. “Antiviral properties of a series of 1,6-naphthyridine and dihydroisoquinoline derivatives exhibiting potent activity against human cytomegalovirus.” Antimicrobial Agents and Chemotherapy. 44:929-937, (2000); Taylor, D., Ahmed, P., Tyms, S., Wood, L., Kelly, L., Chambers, P., Clarke, J., Bedard, J., Bowlin, T., and Rando, R. “Drug resistance and drug combination features of the human immunodeficiency virus inhibitor, BCH-10652 [(±)-2′ deoxy-3′ oxa-4′ thiocytidine, dOTC].” Antimicrobial Chemistry and Chemotherapy 11:291-301, (2000); deMuys, J. M., Gourdeau, H., Nguyen-Ba, N., Taylor, D. L., Ahmed, P. S., Mansour, T., Locas, C., Richard, N., Wainberg, M. A., and Rando, R. F. “Anti-HIV-1 activity, intracellular metabolism and pharmacokinetic evaluation of dOTC (2′-deoxy-3′-oxa-4′-thiocytidine).” Antimicrobial Agents and Chemotherapy 43:1835-1844, (1999); Gu, Z., Wainberg, M. A., Nguyen-Ba, P. L`Heureux, L., de Muys, J.-M., and Rando, R. F., “Mechanism of action and in vitro activity of 1′, 3′-dioxolanylpurine nucleoside analogues against sensitive and drug-resistant human immunodeficiency virus type 1 variants.”]Antimicrobial Agents and Chemotherapy 43:2376-2382, (1999)). In all cases, one should use one or more methods of statistical analysis on the data to discern the degree of synergy, antagonism or strictly additive effects (Chou, T.-C, and P. Talalay “Quantitative analysis of dose-effect relationships: the combined effects of multiple drugs or enzyme inhibitors.” Adv. Enzyme Regul. 22:27-55, (1984); Prichard, M. N., and C. Shipman “A Three-Dimensional Model to Analyze Drug-Drug Interactions.” Antiviral Research 14:181-206., (1990)).

It is also most likely that one will obtain optimal effects on preventing the transmission of HIV when two or more component drugs used in combination each have a unique mechanism of action. This last statement is exemplified in FIG. 5 in which HPMCT was used in combination with the cationic polymer PEHMB. While logically it appears that the negatively-charged polymers like HPMCT or polysulfonates would be a poor choice for inclusion with a cationic compound such as PEHMB (polyethylene hexamethylene biguanide), without wishing to be bound, it is believed that the antiviral activity of PEHMB, and PEHMB-derived molecules, will rely not only upon their charge, but also upon their three-dimensional shape. Therefore it may be possible to obtain mixtures of polyanionic compounds with PEHMB at defined ratios, as seen in FIG. 5. A simple observation of a solution containing 0.25% PEHMB and 0.25% HPMCT in 50 mM Na Citrate pH 6.0 did not detect any undo viscosity, cloudiness or precipitation in the solution indicating that the positive and negative charged species did not interact in a fashion that would cause dissolution (not shown). Further the antiviral activity shown in FIG. 5 determined that the biologic activity of the species was not dampened in any fashion when the two drugs were added simultaneously to the reaction mixture.

It is also possible to mix two or more different negatively charged polymers, copolymers or oligomers together in solution. The utility of this strategy is pronounced when the mechanisms of action of the ingredients are different such as would be the case if HPMCT was added together with a polysulfonated compound such as DS. Cellulosic-based compounds like CAP have been reported to interfere with virus fusion to target cells by blocking co-receptor recognition of the virus, while DS is known to directly block virus attachment to cells via its primary receptor CD4. It is extremely likely that HPMCT and CAT have a mechanism of action similar to CAP.

The experimental design for most combination studies is roughly similar, in that, for each set of two compounds the concentration of one compound is held constant at various points (e.g. the compound's IC25, IC50, IC75 or IC90 value), while the second compound is added to the reaction over a complete range of doses. Then the experiment is performed in reverse, so that the first compound is tested over a complete dose range while the second compound is held steady at one of several concentrations.

Since various classes of chemical agent are being proposed as effective topical therapies for STDs that could not be utilized in systemic therapeutic applications, and these agents could be used effectively with existing systemic therapies for HIV-1, the number of potential combination permutations that could be used for topical applications is greater than that for systemic regimens. For example, as stated above, HPMCT polymers could be used with cationic polymers or oligomers such as PEHMB, with other anionic compounds that have been tried (and failed) clinical trials for systemic applications such as DS, with surfactants such as SDS, or N-9, with known antibiotics, and with the different classes of drugs that have already been approved for systemic treatment of HIV-1. Some examples of the different classes of drugs available or under study are listed in Table 5. All of these examples could be used in combination with the cellulose or acrylic based polymers, copolymers or oligomers of this current invention.

TABLE 5
Classes of agents approved or under consideration for use in human therapy.

| | Mechanism of Action | Drug or drug class |
|---|---|---|
| Virus | | |
| Nucleoside RT Inhibitor | HIV-1 RT Chain Termination | 3TC, Tenofovir, etc. |
| Non Nucleoside RT | RT enzyme inhibition | UC781, CSIC, EFV^{§} |
| Inhibitor | | |
| DNA pol inhibitors (herpes | Viral DNA polymers | Acyclovir, Ganciclovir, |
| viruses) | | Cidofovir, etc. |
| Protease Inhibitor | Protease inhibition | Saquinavir, etc. |
| Fusion Inhibitor HIV-1 | Gp41 trimer formation | T20, CAP, HPMCT, CAT |
| Fusion Inhibitor HSV | | HPMCT, CAP |
| Binding/Fusion Inhibitor | CXCR4 or CCR5 co receptor | T22, AMD3100 |
| | binding inhibitor | |
| Polymers, copolymers or | Binding or fusion inhibition | MVE/MA, Carageenan, DS, |
| oligomers (anionic) | | sulfated dendrimers, |
| | | AR177^{†}, HPMCT, CAT, |
| | | CAP, HPMCP |
| Polymers, copolymers or | — | PEHMB and its variant |
| oligomers (cationic) | | polybiguanides* |
| | HIV-1 Integrase | MK0518, TMC125, |
| | | GS9137 |
| others | | e.g. Ribavirin, interferon |
| Bacterial | | |
| β-lactams | Peptidoglycan cell wall | Penicillins and |
| | synthesis | cephalosporins |
| tetracyclines | | |
| Aminoglycosides | Bacterial | Streptomycin and variations |
| | ribosomes/translation | |
| macrolides | Bacterial | Erythromycin and |
| | ribosomes/translation | variations |
| Fungal | | |
| Polyenes | Disrupt fungal cell wall | Amphotericin B, Nystatin |
| | causing electrolyte leakage | |
| Azoles | Inhibit ergosterol | Fluconazole, Ketoconazole |
| | biosynthesis by blocking 14- | |
| | alpha-demethylase | |
| Allylames | Disrupt ergosteral synthesis | Terbinafine |
| Anti-metabolites | Substrate for fungal DNA | flucytosine |
| | polymerase | |
| Glucan synthesis Inhibitors | Glucan is a key component in | caspofungin |
| | fungal cell wall | |

^{†}AR177 is an effective blocker of virus binding and entry (Este J. A., et al. Mol Pharmacol.; 53(2): 340-5, 1998.
^{§}Motakis, D., and M. A. Parniak “A tight binding mode of inhibition is essential for anti-human immunodeficiency virus type 1 virusidal activity of nonnucleoside reverse transcriptase inhibitors”. Antimicrobial Agents and Chemotherapy 46: 1851-1856, 2002.
*Catalone et al. “Mouse model of cervicovaginal toxicity and inflammation for preclinical evaluation of topical vaginal microbicides.” Antimicrobial Agents. Chemotherapy vol 48, 2004.

### Example 11

Effect of pH on the Antiviral Activity of CAP and HPMCT

The vaginal microenvironment is hard to recapitulate in simple tissue culture systems, but in an attempt to estimate what effects of low pH in the vaginal environment would do to anionic polymer, CAP and HPMCT were rewashed in a low pH buffer before adding the compounds to well-buffered GHOST X4 cells in the presence of H9 cells infected with HIV-1_{SKI} (CD4-dependent cell-associated infection assay). This experiment mimics the effect of exposure to low pH followed by rapid readjustment of the pH in the vaginal lumen by the introduction of semen. The effect of test polymer and the control compound AMD 3100 on virus production was ascertained by monitoring intracellular p24 production 24 hr post-infection.

CD4-dependent HIV Transmission Inhibition Assay

The CD4-dependent HIV transmission inhibition assays use the CD4 positive GHOST(3) X4/R5 or the CD4 positive GHOST(3) R5 cell lines. These cell lines are derived from the HOS (human osteosarcoma) cell line that is negative for HIV coreceptor and CD4 expression. The cell line is engineered to express T4 (CD4), CCR5 and/or CXCR4 via non-selectable retroviral vectors and an HIV-2 LTR hGFP construct with a hygromycin selectable marker.

Twenty-four hours prior to the assay, cells are trypsinized, washed and seeded in 96-well flat bottom microtiter plates. On the day of the assay, effector cells (H9 cells chronically infected with the SKI clinical isolate of HIV-1, or MOLT4 cells chronically infected with the JR-CSF molecular clone) are treated with freshly made mitomycin C (200 μg/ml) for 60 minutes at 37° C. This concentration of mitomycin C is sufficient to result in cell death, but allows virus transmission to occur. After mitomycin C treatment, the effector cells are washed repeatedly with tissue culture medium. Test compounds are added to the monolayer followed by effector cells. The cells are co-cultured with effecter cells and test material for 4 hours, and the effector cells are removed by washing the monolayer repeatedly with RPMI. At 20 hours after assay initiation, the wells are again washed to ensure removal of the effecter cells, and virus replication is assessed via measurement of cell-associated HIV-1 gag p24 using an ELISA (Beckman-Coulter p24 ELISA). Compound toxicity and cell viability are assessed by MTS dye reduction.

Compounds evaluated in the pH transition assay are set up essentially the same as described above, with the exception that compounds are prepared in medium adjusted to a pH of 3.45 to 6.5 before addition to well-buffered target cells. Addition of effector cells prepared in a buffered medium results in a transition of the pH to near neutrality. All determinations are performed in triplicate with serial Log₁₀ dilutions of the test materials.

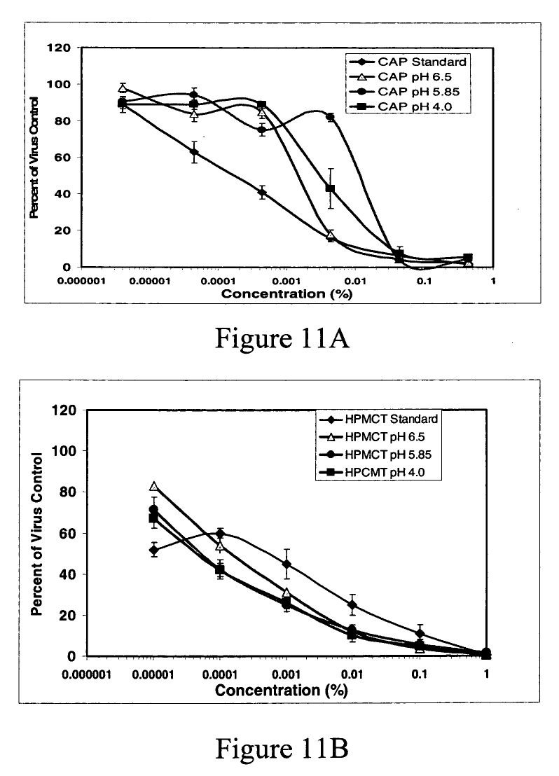

In this experiment, both compounds exhibited anti-viral activity, but the antiviral activity of CAP and HPMCT were diminished to a greater degree by the low pH treatment than was that of HPMCT (FIG. 11A and 11B). In fact, the adjustment of the preincubation conditions from a standard assay condition of pH approximately 7, to a lower pH (between 4 to 6.5) seemed to slightly enhance the activity of HPMCT. In this set of experiments, the CXCR4 chemokine receptor antagonist AMD3100 was used as a positive control. It is interesting to note that the activity of AMD 3100 also increased upon preincubation at low pH, as shown in table 6 (Table 6).

TABLE 6
pH effect on pH or CD4 dependent transmission of HIV-1_{SKI}.

| | Pre-incubation | | | Therapeutic |
|---|---|---|---|---|
| Compound | pH* | IC₅₀ | CC₅₀ | Index |
| CAP | Standard Assay | 0.0002% | >0.40% | >2,175 |
| HPMCT | Standard Assay | 0.0005 | 0.39% | 784 |
| AMD 3100 | Standard Assay | 0.01 μM | >10.0 μM | >1,000 |
| CAP | 4.0 | 0.003% | 0.38% | 127 |
| HPMCT | 4.0 | 0.00005% | 0.38% | 7,580 |
| AMD 3100 | 4.0 | 0.001 μM | 4.03 μM | 4.030 |
| CAP | 5.85 | 0.01% | >0.40% | >40.0 |
| HPMCT | 5.85 | 0.00005% | 0.38% | 7,700 |
| AMD 3100 | 5.85 | 0.006 μM | >10.0 μM | >1,667 |
| CAP | 6.5 | 0.001% | 0.20% | 200 |
| HPMCT | 6.5 | 0.0001% | 0.39% | 3,920 |
| AMD 3100 | 6.5 | 0.08 μM | >10.0 μM | >125 |

*There was no pre-incubation of test compound using the standard assay. These data represent the averages of three or more independent experiments. The Standard Deviation was obtained at each polymer concentration tested and ranged between 0.4 and 7% of the data points normalized to percent viral inhibition or percent cell viability.

In addition, the increase in antiviral activity of HPMCT and AMD3100 did not correspond to a similar magnitude change in the overall toxicity of these compounds; therefore, the overall effect of the preincubation was a net increase in the therapeutic indices for these two compounds (Table 7).

### Example 12

A variation of the pH transition assay described above was employed to try to more closely mimic the crucial events that occur upon initial exposure to HIV-1. In this experiment, cell-associated HIV-1 (H9 cells infected with HIV-1_{SKI}) was used to infect the cervical epithelia cell line ME 180 (CD4-independent assay). The infection assay itself, as described for the CD4-dependent assay, is carried out under neutral culture conditions, and we used DS as a control. Viral p24 levels in the supernatant were determined 6 days post-infection.

CD4-independent HIV Transmission Inhibition Assay.

In this assay, a cervical epithelia cell line (ME-180) that has been adapted to survive at pH 4.5 for 4 hr was used. H9 cells chronically infected with HIV-1_{SKI} are added in the presence of a test polymer which helps to buffer the entire mixture back into the neutral range as described for the CD4-dependent assays. Target cells are washed after 4 hr and again after 24 and 48 hr post-infection, and the culture is maintained for 6 days, at which time the culture supernatants are collected and assayed for the presence of HIV-1 gag p24 antigen by ELISA.

Viral p24 Antigen ELISA.

ELISA kits are purchased from Coulter, and detection of supernatant or cell-associated p24 antigen is performed according to the manufacturer's instructions or as previously described (8, 14). For cell-associated p24, cell lysates are prepared by lysis of the well contents in 25 to 100 μl of lysis buffer, and assayed following 1 round of freeze/thaw. All p24 determinations are performed following serial dilution of the samples to ensure absorbance values in the linear range of the standard p24 antigen curve. The standard curve is generated using manufacturer-supplied standards and instructions. Data are obtained by spectrophotometric analysis at 450 nm using a Molecular Devices Vmax or SpectraMaxPlus plate reader. Final concentrations are calculated from linear regression analysis of the optical density values and expressed in pg/ml p24 antigen.

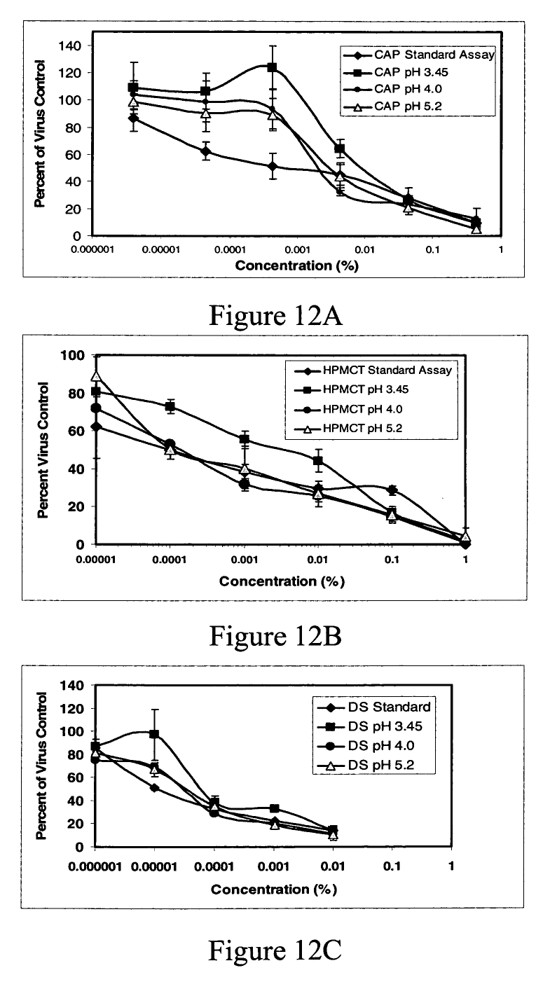

The results are shown in Table 7 and FIG. 12.

TABLE 7
Effect of pH on CD4-independent transmission of HIV-1_{SKI}.

| | Pre-incubation | | | Therapeutic |
|---|---|---|---|---|
| Compound | pH* | IC₅₀ | CC₅₀ | Index |
| CAP | Standard Assay | 0.001% | >0.40% | >400 |
| HPMCT | Standard Assay | 0.0001% | 0.35% | 3500 |
| DS | Standard Assay | 0.00001% | >0.01% | >1,000 |
| CAP | 3.45 | 0.01% | >0.40% | >40 |
| HPMCT | 3.45 | 0.003% | 0.47% | 157 |
| DS | 3.45 | 0.00006% | >0.01% | >167 |
| CAP | 4.0 | 0.002% | >0.40% | >200 |
| HPMCT | 4.0 | 0.0001% | 0.37% | 3,700 |
| DS | 4.0 | 0.00003% | >0.01% | >333 |
| CAP | 5.2 | 0.003% | >0.40% | >133 |
| HPMCT | 5.2 | 0.0001% | 0.27% | 2,700 |
| DS | 5.2 | 0.000035% | >0.01% | >286 |

*There was no pre-incubation of test compound using the standard assay. These data represent the averages of three or more independent experiments. The Standard Deviation was obtained at each polymer concentration tested and ranged between 1.0 and 12% of the data points normalized to percent viral inhibition or percent cell viability.

In this assay, all three polyanions tested were negatively affected when the pH of the pre-incubation buffer was below 4.0. (See FIG. 12 and Table 7). However, at pH 4.0 or 5.2, there was no detectable change in the activity of HPMCT when compared to the standard assay conditions. This is readily apparent when comparing the IC₅₀s obtained (Table 7) or by observation of the dose-response curves (FIG. 12A). At these same pH values, there was a marked decrease in the antiviral activity of CAP, and to a lesser extent DS, at all three low pH preincubation conditions. Nevertheless, they all had some activity, even at the low pH's.

### Example 13

Effect of Acid Substitution Pattern on Antiviral Activity.

Having determined that trimellitate-containing polymers maintained their antiviral activity even after exposure to low pH conditions, an investigation was conducted on whether the degree of acid substitution could affect the overall anti-HIV-1 profile of a selected polyanion. The first, and most basic, assay employed was the virus attachment assay, described above, and this time both a CXCR4 tropic strain of HIV-1 (strain IIIB) and a macrophage/monocyte CCR5 tropic strain (BaL) of HIV-1 were used to infect either HeLa CD4 LTR β-gal cells or MAGI-R5 cells. At the end of the exposure period, cells were washed and further incubated in virus- and compound-free media for 40 to 48 hr. Compound toxicity was monitored in parallel.

Virus Attachment Assay.

This assay is designed to detect compounds that block virus attachment using MAGI-R5 or HeLa CD4 LTR β-gal cells. Twenty-four hours prior to initiation of the assay, the cells are trypsinized, counted and plated in a 0.2 cm well in media without selection antibiotics. After 24 hr, media is removed and fresh media-containing test compound is placed on the cells and incubated for 15 min at 37° C. A known titer of HIV-1_{IIIB} or HIV-1_{BaL} is then added to the wells and the incubation is continued for 2 to 4 hr. At the end of the incubation, the wells are washed 2 times with media and the culture is continued for 40 to 48 hr. At termination of the assay, media is removed and β-galactosidase enzyme expression is determined by chemiluminescence per manufacturer's instructions (Tropix Gal-screen™, Bedford, Mass.). Compound toxicity is monitored on a sister plate by XTT or MTS dye reduction. All determinations are performed in triplicate with serial ½ Log₁₀ dilution of the test materials. The virus adsorption interval of 1 to 2 hr is sufficiently short that AZT, which requires intra-cellular phosphorylation to achieve its active tri-phosphate form (AZT-TTP), is not active in this assay. The results are tabulated in Table 8.

TABLE 8
Effect of trimellityl content on anti-HIV-1 activity of HPMCT in a virus
attachment assay.

| | Assay Virus HIV-1_{IIIB} | | | | Assay Virus HIV-1_{BaL} | | | |
|---|---|---|---|---|---|---|---|---|
| Compound* | IC₅₀ | IC₉₀ | CC₅₀ | TI₅₀ | IC₅₀ | IC₉₀ | CC₅₀ | TI₅₀ |
| TAK 779 | >200 | >200 | >200 | NA | <0.002 | 0.04 | >200 | >100,000 |
| AMD 3100 | 0.001 | 0.008 | >10 | >10,000 | 3.79 | >10 | >10 | >2.64 |
| CAP-35^{†} | 0.007 | 0.03 | >25 | >3,500 | 0.004 | 0.11 | >25 | >6,250 |
| HPMCT-29 | 0.005 | 0.03 | >25 | >5,000 | 0.069 | 2.18 | >25 | >40 |
| HPMCT-35 | 0.001 | 0.01 | 10.4 | 10,400 | 0.023 | 0.14 | 10.3 | 68.67 |
| HPMCT-36 | <0.0003 | 0.0002 | 10 | >33,333 | 0.03 | 0.44 | >25 | >800 |
| HPMCT-37^{§} | 0.0003 | 0.002 | 10.8 | 35,643 | 0.08 | 0.22 | >25 | >300 |
| HPMCT-41% | <0.0003 | 0.001 | 11.1 | >37,000 | 0.0031 | 0.22 | >25 | >625 |
| HPMCT-49% | <0.0003 | 0.001 | 8.80 | >29,333 | 0.006 | 0.12 | 9.91 | 1,651 |
| DS | <0.0001 | — | >2 | >20,000 | 0.007 | — | >2 | >285 |

*All efficacy and toxicity values for TAK 779 are in nM; for AMD 3100 are in μM; and for anionic polymers are in weight percent in the aqueous solution. These data represent the averages of three or more independent experiments. The Standard Deviation was obtained at each polymer concentration tested and ranged between 0.6 and 8% of the data points normalized to percent viral inhibition or percent cell viability.
^{†}The percentage of benzoic acid (phthalate or trimellitate) substituted per total dry weight of polymer.
^{§}The average molecular weight for HPMCT-37 is 30 kD, for all other polymers it is ˜50 kD.

In this assay, all test compounds were less active against HIV-1_{BaL} (CCR5 tropic) than against HIV-1_{IIIB} (CXCR4 tropic), including the polyanion control, DS (See data in Table 8). The ability of HPMCT to inhibit CCR5 tropic virus in this assay system increased with increasing degree of trimellityl substitution on the cellulose backbone. It is interesting to note that HPMCT-37 (37 weight percent trimellityl substitution), which has an average molecular weight of 30 kD, as compared to the average molecular weight of ˜50 kD for the other HMCT polymers tested, was less efficacious in all test systems used.

HPMCT variants were further tested for their ability to retard cell-associated HIV-1 transmission. In these experiments, CD4 positive GHOST cells were used as target cells and were incubated with cell-associated viruses. The results are shown in Table 9.

TABLE 9
Effect of Trimellityl content on cell-associated HIV-1 infectivity.

| | CXCR4 Virus HIV-1_{SKI}^{§} | | | | Assay Virus HIV-1_{JR-CSF}^{§} | | | |
|---|---|---|---|---|---|---|---|---|
| Compound* | IC₅₀ | IC₉₀ | CC₅₀ | TI₅₀ | IC₅₀ | IC₉₀ | CC₅₀ | TI₅₀ |
| TAK 779 | >10 | >10 | >10 | NA | 0.002 | 0.55 | >10 | >10,000 |
| AMD 3100 | 0.005 | 0.06 | >10 | >2,000 | >10 | >10 | >10 | NA |
| CAP-35^{†} | 0.06 | 7.45 | >25 | >415 | 2.93 | >25 | >25 | >8.50 |
| HPMCT-29 | >25 | >25 | >25 | NA | 12.7 | >25 | >25 | >1.97 |
| HPMCT-35 | 0.18 | 13.7 | 9.7 | 53.89 | 7.82 | 19.8 | 9.07 | 1.16 |
| HPMCT-36 | 0.18 | >25 | >25 | >138 | 8.22 | >25 | >25 | >3 |
| HPMCT-37 | 0.76 | 23.2 | 15.5 | 20.39 | 5.5 | 18.5 | 19.6 | 3.56 |
| HPMCT-49 | 0.00025 | 2.61 | 9.32 | 37,200 | 0.006 | 0.12 | 9.91 | 1,651 |

*All efficacy and toxicity values for TAK 779 are in nM; for AMD 3100 are in μM; and for anionic polymers are in weight percent polymer. These data represent the averages of three or more independent experiments. The Standard Deviation was obtained at each polymer concentration tested and ranged between 0.6 and 8% of the data points normalized to percent viral inhibition or percent cell viability.
^{†}The percentage of benzoic acid substituted per total dry weight of polymer.
^{§}The average molecular weight for HPMCT-37 is 30 kD, for all other polymers it is ˜50 kD.

The results show that like the virus attachment assay described in Table 9, all of the polymers tested were less effective against the CCR5 tropic strain of virus than the CXCR4 tropic strain (See results in Table 9).

In addition, as seen in the virus attachment assay, the most heavily substituted variant of HPMCT tested (HPMCT-49) was clearly superior to all other polymers tested against both CXCR4 and CCR5 tropic strains of virus.

### Example 14

The ability of HPMCT to interfere with HIV infection or replication was next assessed using PBMCs infected with either a CXCR4 tropic (CMU06), a CCR5 tropic (JRCSF), or a dual tropic (BR/92/014) strain of HIV-1. In these experiments the virus was added to cells in the presence of test compound for seven days. The protocol is as follows:

HIV-1 Infection and Replication in Peripheral Blood Mononuclear Cells (PBMCs).

Fresh human PBMCs, seronegative for HIV and HBV, were isolated from screened donors using lymphocyte separation Medium (LSM, Cellgro® by Mediatech, Inc.; density 1.078+/−0.002 g/mL).

PHA-P stimulated cells from at least two normal donors were pooled, diluted in fresh media and plated in a 96-well round bottom microplate. Test compound dilutions were prepared and added to the cells, and then a predetermined dilution of virus stock was placed in each test well at a final MOI of approximately 0.1. The PBMC cultures were maintained for seven days following infection at 37° C., 5% CO₂ at which time cell-free supernatant samples were obtained and tested for HIV-1 RT activity.

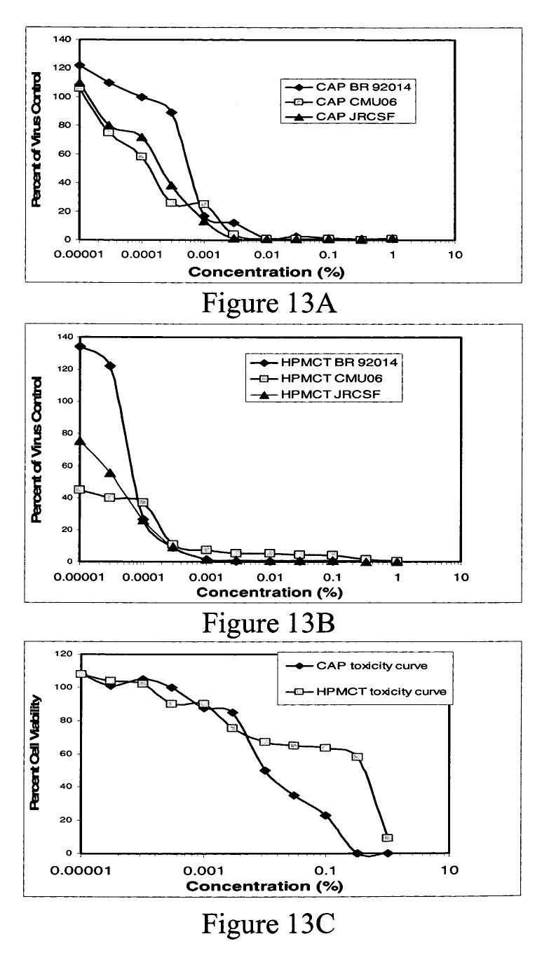

The results are provided in FIG. 13 they show how over this extended period of exposure the efficacy of HPMCT-49 increased relative to that observed in the shorter duration exposures (FIG. 13). The calculated IC₅₀ for this HPMCT variant against the CXCR4 strain of HIV-1 was <0.00001%, against the CCR5 strain of virus was 0.00004%, and against the dual tropic strain was 0.00008%. The toxicity of the compounds also increases with the increased exposure time, but the resulting therapeutic indices obtain in all cases were >3500. AZT was used as a positive control in this experiment and the IC₅₀s calculated for this compound were <0.1, 1.48 and 0.27 nM for the CXCR4, CCR5 and dual tropic viruses respectively.

### Example 15

Inhibition of Viral Mediated Fusion Events.

Without wishing to be bound, it is believed that the mechanism of action for all polyanions is believed to be via interference with the events by which the virus attaches to, and fuses with, the target cell membrane. The fusion assay employed assesses the ability of compounds to block cell-to-cell fusion mediated by HIV-1 envelope glycoprotein and CD4 expressed on separate cells. The assay hereinabove is sensitive to inhibitors of both the gp120/CD4 interaction and the gp120/CXCR4 coreceptor interaction.

Fusion Assay.

The fusion assay assesses the ability of compounds to block cell-to-cell fusion mediated by HIV-1 envelope glycoprotein and CD4 expressed on separate cells. This assay is sensitive to inhibitors of both the gp120 interaction with cellular CD4 and the CXCR4 coreceptor. HeLa CD4 LTR β-gal cells are plated in microtiter wells and diluted compounds are added and allowed to incubate at 37° C. for 1 hr prior to the addition of HL2/3 cells. The incubation is then continued for 40 to 48 hr, after which fusion is monitored by measurement of β-galactosidase enzyme expression, detectable by chemiluminescence (Tropix Gal-screen™, Bedford, Mass.). Compound toxicity is monitored on a sister plate using XTT or MTS dye reduction. All determinations are performed in triplicate with serial ½ Log₁₀ dilutions of the test materials.

The results from this experiment are presented in Table 10 hereinbelow.

TABLE 10
Effect of trimellityl content on virus mediated fusion events^{§}.

| Compound | IC₅₀ | IC₉₀ | CC₅₀ | TI₅₀ |
|---|---|---|---|---|
| AMD 3100 | 0.002 | 0.007 | >10 | >5,000 |
| (μM) | | | | |
| TAK 779 (nM) | >200 | >200 | >200 | NA |
| CAP-35 (%) | 0.01 | 0.13 | 18.4 | 1,840 |
| HPMCT-35 (%) | 0.06 | 0.23 | 12.9 | 215 |
| HPMCT-41 (%) | 0.01 | 0.08 | 11.4 | 1,140 |
| HPMCT-49 (%) | 0.001 | 0.002 | 11 | 11,000 |

^{§}These data represent the averages of three or more independent experiments. The standard deviation was obtained at each polymer concentration tested and ranged between 2.0 and 6.0% of the data points normalized to percent viral inhibition or percent cell viability.

The data clearly show that HPMCT, like CAP, is capable of interfering with binding or fusion events. In addition, as seen in the viral inhibition studies, the degree of trimellityl substitution directly correlates to the degree of fusion inhibition.

### Example 16

Effect of Benzoic Acid-Containing Polymers on Lactobacillus Growth:

Lactobaccilli are naturally occurring and beneficial constituents of the vaginal microenvironment, and while it is helpful to have some degree of broad action against STD pathogens, it would be optimal for said agent to not compromise the natural flora. For this reason, the effect of HPMCT on L. crispatus and L. Jensenii growth was generated and assessed.

Lactobacillus Assay.

Lactobacillus crispatus and Lactobacillus jensenii were obtained from the American Type Culture Collection and grown in Lactobacilli MRS broth (Difco, Fisher Scientific, Pittsburgh, Pa.). This medium allows efficient growth of the Lactobacilli under facultative anaerobic conditions. Bacterial stocks are produced and frozen in 15% glycerol at −80° C. for use in the sensitivity assay. To assess the effect of compounds on L. crispatus and L. jensenii growth, 10 ml of MRS media is inoculated with a stab from the glycerol bacterial stock and the culture is incubated for 24 hr at 37° C. The next day, the bacterial density is adjusted to an OD of 0.06 at a wavelength of 670 nm. Compounds are diluted and dispensed into 96-well round bottomed plates and the diluted Lactobacillus spp. is added. Commercially-available penicillin/streptomycin solution at a high-test concentration of 1.25 U/ml and 1.25 μg/ml, respectively, is used as the positive control. The plates are incubated for 24 hr at 37° C. in a Gas Pak CO₂ bag, and bacterial growth is determined by measurement of optical density at 490 nm using a 96-well spectrophotometric plate reader. All determinations are performed with six ½ Log dilutions from a high test concentration in triplicate.

The data obtained are depicted in Table 11:

TABLE 11
Effect of Trimellityl content on cell-associated HIV-1 infectivity.

| | CXCR4 Virus HIV-1_{SKI}^{§} | | | | Assay Virus HIV-1_{JR-CSF}^{§} | | | |
|---|---|---|---|---|---|---|---|---|
| Compound* | IC₅₀ | IC₉₀ | CC₅₀ | TI₅₀ | IC₅₀ | IC₉₀ | CC₅₀ | TI₅₀ |
| TAK 779 | >10 | >10 | >10 | NA | 0.002 | 0.55 | >10 | >10,000 |
| AMD 3100 | 0.005 | 0.06 | >10 | >2,000 | >10 | >10 | >10 | NA |
| CAP-35^{†} | 0.06 | 7.45 | >25 | >415 | 2.93 | >25 | >25 | >8.50 |
| HPMCT-29 | >25 | >25 | >25 | NA | 12.7 | >25 | >25 | >1.97 |
| HPMCT-35 | 0.18 | 13.7 | 9.7 | 53.89 | 7.82 | 19.8 | 9.07 | 1.16 |
| HPMCT-36 | 0.18 | >25 | >25 | >138 | 8.22 | >25 | >25 | >3 |
| HPMCT-37 | 0.76 | 23.2 | 15.5 | 20.39 | 5.5 | 18.5 | 19.6 | 3.56 |
| HPMCT-49 | 0.00025 | 2.61 | 9.32 | 37,200 | 0.006 | 0.12 | 9.91 | 1,651 |

*All efficacy and toxicity values for TAK 779 are in nM; for AMD 3100 are in μM; and for anionic polymers are in weight percent polymer. These data represent the averages of three or more independent experiments. The Standard Deviation was obtained at each polymer concentration tested and ranged between 0.6 and 8% of the data points normalized to percent viral inhibition or percent cell viability.
^{†}The percentage of benzoic acid substituted per total dry weight of polymer.
^{§}The average molecular weight for HPMCT-37 is 30 kD, for all other polymers it is ˜50 kD.

The data indicate that all polyanions tested were relatively ineffective as inhibitors of lactobacilli growth. The selectivity index between the concentration needed to inhibit 50% Lactobacilli growth and that needed to inhibit HIV-1 becomes larger than that obtained when using cellular cytotoxicity as the numerator. In addition, the bacterial inhibition assay is set up to allow for a 24 hr exposure to test compound, which is not a likely regimen for human use.

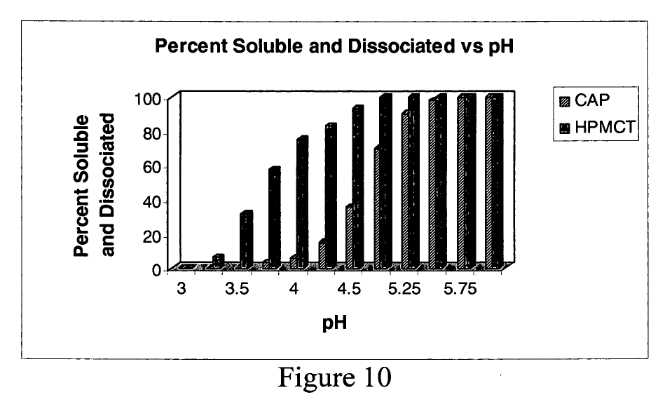

The data illustrate hereinabove, in a variety of assay formats, that HPMCT polymer is quite effective at inhibiting HIV-1 and that the extent of inhibition can be modulated by the degree of trimellityl substitution on the cellulose backbone. What separates HPMCT polymer from the similar cellulose-based polymers (CAP and HPMCP polymers) is its ability to remain dissociated in solution and molecularly dispersed even after exposure to a low pH environment. For example, while all four cellulose-based polymers tested were effective inhibitors of HIV-1IIB after a short duration of exposure using assay conditions that were basically neutral (Table 2), the exposure of CAP to a low pH environment for even a brief period of time dramatically lowered its antiviral effectiveness (Tables 6 and 7). The effect of low pH on phthalate-containing polymers was further visualized by monitoring the solubility and dissociation of CAP over a wide range of pH conditions (FIG. 10). Monitoring the combined effect of both solubility and dissociation on CAP, we determined that less than 10% of the original polymer is available when the pH drops to 4.0 before readjusting to neutral. The reason that not more CAP is available once the pH has been rapidly neutralized under these assay conditions is simply due to the long dissolution time of these polymers once they have fallen out of solution.

The data further show that there was a measurable differential between activity against HIV-1_{IIIB}(CXCR4) and HIV-1_{BaL} (CCR5) when compounds were tested using a virus attachment assay (Table 9). In these experiments, DS was clearly able to inhibit HIV-1_{BaL}, albeit at a reduced level as was the case for all compounds tested against this strain of virus (Table 9). Without wishing to be bound, it is believed that the real differences in activity arose when the compounds were tested in a cell-associated transmission assay (Table 10). It should be noted that the degree of change in trimellityl-containing polymers roughly correlated with the degree of carboxylic acid substitution. With the comparison of the different HPMCT lots that minor variations in the degree of trimellityl substitution has a dramatic impact on the antiviral efficacy of the polymer, especially with respect to its activity against CCR5 virus (Tables 9, 10, and 11).

Without wishing to be bound, it is believed that the overall average molecular weight of the polymer can also play a role in their antiviral efficacy, as noted for HPMCT-37 (average molecular weight 30 kD).

### Examples 17-18

The ability of additional compounds of the present invention to inhibit additional viral strains were determined. In the following examples, the antiviral profile of PSMA (poly styrene alt maleic acid) and hydroxy propyl methycellulose trimellitate (HPMCT), both of which are prepared as described herein were determined on various viral strains.

Various assays were utilized, the protocol of which are described below.

1. VBI (Virus Attachment Inhibitor Assay)

The protocol was described in Example 13, the contents of which are incorporated by reference.

2. Rapid Screening Assay

When relatively large numbers (10 or more) of test compounds are submitted at the same time from a single sponsor, the compounds are evaluated in a 2-concentration test. In this procedure, 2 concentrations (200, 20 μg/ml unless otherwise directed) are tested. These are diluted 1:2 when virus is added, making final concentrations 100 and 10 μg/ml. The standard CPE test uses an 18 h monolayer (80-100% confluent) of the appropriate cells, medium is drained and each of the concentrations of test compound or placebo are added, followed within 15 min by virus or virus diluent. Two wells are used for each concentration of compound for both antiviral and cytotoxicity testing.

The plate is sealed and incubated the standard time period required to induce near-maximal viral CPE. The plate is then stained with neutral red by the method described below and the percentage of uptake indicating viable cells read on a microplate autoreader at dual wavelengths of 405 and 540 nm, with the difference taken to eliminate background. An approximated virus-inhibitory concentration, 50% endpoint (EC50) and cell-inhibitory concentration, 50% endpoint (IC50) will be determined from which a general selectivity index is calculated: SI=(IC50)/(EC50). An SI of 3 or greater indicates confirmatory testing is needed.

3. Standard Assay: Inhibition of Viral Cytopathic Effect (CPE)

This test, run in 96 well flat-bottomed microplates, is used for the initial antiviral evaluation of all new test compounds. In this CPE inhibition test, four log₁₀ dilutions of each test compound (e.g. 1000, 100, 10, 1 μg/ml) is added to 3 cups containing the cell monolayer; within 5 min, the virus is then added and the plate sealed, incubated at 37° C. and CPE read microscopically when untreated infected controls develop a 3 to 4+ CPE (approximately 72 to 120 hr). A known positive control drug is evaluated in parallel with test drugs in each test. This drug is Ribavirin for dengue, influenza, measles, respiratory syncytial, parainfluenza, Pichinde, Punta Toro and Venezuelan equine encephalitis viruses, cidofovir for adenovirus, pirodovir for rhinovirus, 6-azauridine for West Nile and yellow fever viruses, and alferon (interferon alfa-n3) for SARS virus.

Follow-up testing with compounds found active in initial screening tests are run in the same manner except 8 one-half log₁₀ dilutions of each compound are used in 4 cups containing the cell monolayer per dilution.

The data are expressed as 50% effective concentrations (EC50).

4. Standard Assay: Increase in Neutral Red (NR) Dye Uptake

This test is run to validate the CPE inhibition seen in the initial test, and utilizes the same 96-well micro plates after the CPE has been read. Neutral red is added to the medium; cells not damaged by virus take up a greater amount of dye, which is read on a computerized micro plate autoreader.

The method as described by McManus (Appl. Environment. Microbiol. 31:35-38, 1976), the contents of which are incorporated by reference, is used. An EC50 is determined from this dye uptake.

5. Decrease in Virus Yield Assay (VYA).

Compounds considered active by CPE inhibition and by NR dye uptake are re-tested if additional, fresh material is available, using both CPE inhibition and, using the same plate, effect on reduction of virus yield by assaying frozen and thawed eluates from each cup for virus titer by serial dilution onto monolayers of susceptible cells. Development of CPE in these cells is the indication of presence of infectious virus. As in the initial tests, a known active drug is run in parallel as a positive control. The 90% effective concentration (EC90), which is that test drug concentration that inhibits virus yield by 1 log10, is determined from these data.

6. Methods for Assay of Cytotoxicity

A. Visual Observation

In the CPE inhibition tests, two wells of uninfected cells treated with each concentration of test compound are run in parallel with the infected, treated wells. At the time CPE is determined microscopically, the toxicity control cells are examined microscopically for any changes in cell appearance compared to normal control cells run in the same plate. These changes may be enlargement, granularity, cells with ragged edges, a filmy appearance, rounding, detachment from the surface of the well, or other changes. These changes are given a designation of T (100% toxic),

PVH (partially toxic-very heavy-80%), PH (partially toxic-heavy-60%), P (partially toxic40%), Ps (partially toxic-slight-20%), or 0 (no toxicity-0%), conforming to the degree of cytotoxicity seen. A 50% cell inhibitory (cytotoxic) concentration (IC50) is determined by regression analysis of these data.

B. Neutral Red Uptake

In the neutral red dye uptake phase of the antiviral test described above, the two toxicity control wells also receive neutral red and the degree of color intensity is determined spectrophotometrically. A neutral red IC50 (NR IC50) is subsequently determined. The IC50 is also commonly referred to as the CC50 or concentration needed to reduce cell viability by 50%.

C. Viable Cell Count

Compounds considered to have significant antiviral activity in the initial CPE and NR tests are re-tested for their effects on cell growth. In this test, 96-well tissue culture plates are seeded with cells (sufficient to be approximately 20% confluent in the well) and exposed to varying concentrations of the test drug while the cells are dividing rapidly. The plates are then incubated in a CO2 incubator at 37° C. for 72 hr, at which time neutral red is added and the degree of color intensity indicating viable cell number is determined spectrophotometrically; an IC50 is determined by regression analysis.

### Example 17

The antiviral activity of PSMA on various viruses was conducted utilizing the procedures described hereinabove. The results are indicted hereinbelow in Table 12:

TABLE 12
Antiviral Profile of Polystyrene alt Maleic Acid (PSMA).

| Virus/Strain | Assay | IC₅₀ (%) | CC₅₀ (%) | TI CC5₅₀/IC₅₀ |
|---|---|---|---|---|
| HIV-1 IIIB | VBI | 0.0009 | 3.2 | 3555 |
| CXCR4 tropic | | | | |
| HIV-1 BaL | VBI | 0.001 | 3.2 | 3200 |
| CCCR5 tropic | | | | |
| HSV1 | CPE | 0.002 | >0.2 | >100 |
| HSV2 | CPE | 0.006 | >0.2 | >33 |
| Punta Toro- | Neutral Red | 0.00043 | 0.069 | 160 |
| Adames | | | | |
| SARS- | Neutral Red | 0.07 | >10 | >140 |
| URBANI | | | | |
| Influenza A | Neutral Red | <0.00007 | 0.051 | >700 |
| H1N1 New | and VYA | | | |
| Calidonia/20/99 | | | | |
| Influenza A | Neutral Red | <0.000048 | <0.048 | >1000 |
| H3N2 | and VYA | | | |
| California/7/04 | | | | |
| Influenza | Neutral Red | <0.000053 | <0.053 | >1000 |
| H5N1A | and VYA | | | |
| Influenza B | Neutral Red | <0.000048 | <0.048 | >1000 |
| | and VYA | | | |
| RSV A (A2) | Neutral Red | 0.0005 | >0.05 | >100 |
| | Visual count | 0.0009 | >1 | >1000 |

VBI is a viral binding inhibition assay or virus attachment inhibition assay. CPE is a cytophathic effect assay. Neutral red monitors changes in neutral red (dye) uptake in cells that are either infected with virus or in uninfected controls. VYA is a virus yield reduction assay.

### Example 18

The activity of various HPMCTs an various viruses were conducted using the procedures described hereinabove. See for example, Kokubo et al., Chem Pharm Bull, 45:1350-1353 (1997), the contents of which are incorporated by reference. The results are indicated in Table 13.

TABLE 13
Antiviral Profile of hydroxypropyl methylcellulose
trimellitate (HPMCT).

| Compound | Virus/Assay | IC₅₀ (%) | CC₅₀ (%) | TI CC5₅₀/IC₅₀ |
|---|---|---|---|---|
| HPMCT-35 | HIV-1 IIIB | 0.001 | 10.4 | 10,400 |
| | CXCR4 tropic- | | | |
| | VBI | | | |
| HPMCT-35 | HIV-1 BaL | 0.023 | 10.3 | 447 |
| | CCCR5 tropic- | | | |
| | VBI | | | |
| HPMCT 49 | HIV-1 IIIB | <0.0003 | 8.8 | >29,000 |
| | CXCR4 tropic- | | | |
| | VBI | | | |
| HPMCT-49 | HIV-1 BaL | 0.006 | 9.91 | 1,651 |
| | CCCR5 tropic- | | | |
| | VBI | | | |
| HPMCT-35 | HSV1-CPE | 0.004 | >0.08 | >20 |
| HPMCT-35 | HSV2-CPE | 0.004 | >0.08 | >20 |
| HPMCT-49 | HSV1-CPE | <0.0006 | >0.08 | >133 |
| HPMCT-49 | HSV2-CPE | 0.001 | >0.08 | >80 |
| HPMCT-35 | cowpox-CPE | 0.0017 | >0.03 | >17 |
| HPMCT-35 | vaccina-CPE | 0.0035 | >0.03 | >12 |
| HPMCT-49 | cowpox-CPE | 0.0012 | >0.03 | >25 |
| HPMCT-49 | vaccina-CPE | 0.00049 | >0.03 | >60 |
| HPMCT-35 | RSV A2- | 0.002 | >0.05 | >25 |
| | Neutral Red | | | |
| HPMCT-35 | RSV A2-Visual | 0.01 | >0.1 | >10 |
| | Confirmation | | | |
| HPMCT-49 | RSV A2- | <0.00005 | 0.04 | 800 |
| | Neutral Red | | | |
| HPMCT-49 | RSV A2-Visual | 0.001 | >0.1 | >100 |
| | Confirmation | | | |

HPMCT-35 and HPMCT-49 contain 35 and 49 weight percent of trimellitic acid respectively.
VBI is a viral binding inhibition assay or virus attachment inhibition assay. CPE is a cytophathic effect assay. Neutral red monitors changes in neutral red (dye) uptake in cells that are either infected with virus or in uninfected controls. VYA is a virus yield reduction assay.

### Example 19

Using the assays described hereinabove, the antiviral activity of three of the compounds described herein were tested.

Initial screening (viral cytopathic effect or CPE assay) was performed using 96-well flat bottomed microplates, in which four log₁₀ dilutions of each test compound were added to 3 replica wells containing a target cell monolayer; within 5 minutes, the test virus was added and the plate sealed, incubated at 37° C. and CPE read microscopically when untreated infected controls developed a 3 to 4+ CPE (approximately 72 to 120 hr). A known positive control drug was evaluated in parallel with each test compound. Follow-up testing for compounds found active in initial screening tests were run in the same manner, except 8 one-half log₁₀ dilutions of each compound were used in 4 replica wells containing the cell monolayer per dilution.

The results of the initial virus screening were quite surprising in that a large degree of antiviral specificity was observed for the polymers tested. See Table 14.

TABLE 14
Virus Screening Panel Results.

| | Test Polymer** | | |
|---|---|---|---|
| | Therapeutic Index (CC₅₀/IC₅₀) | | |
| Virus | HPMCT | MVE/MA | PSMA |
| HIV-1_{IIIB} | >5000 | ˜1000 | >1000 |
| HSV1 | >133 | >16 | >100 |
| HSV2 | >80 | >2 | >33 |
| VZV | 0 | 0 | 0 |
| HHV-6A | 35 | 19 | 1.7 |
| HHV-6B | 4 | 0 | 0 |
| Cowpox | >17.7 | 0 | >1.5 |
| Vaccinia | >17.7 | 0 | >2 |
| PIV | 1 | 1 | 3 |
| SARS | 1 | 0 | >187 |
| Influenza | >2 | >3 | >1300 |
| RSV | >100 | 0 | >100 |
| Punta Toro | | | >100 |
| Rift Valley Fever | | | 100 |
| VEE | >400 | | >2000 |

*The HIV-1 assay employed was designed to monitor inhibition of virus transmission; All other assays were variation of a CPE method.
**PEHMB = polyethylene hexamethylene bis biguanide; PEHMG = polyethylene hexamethylene guanidine; HPMCT = hydroxypropyl methylcellulose trimellitate; MVE/ME = methyl vinyl ether alt with maleic acid; PSMA = Poly (styrene alt maleic acid)

### Example 20

Additional testing was performed against a number of different strains of influenza, including murine adapted strains. The assays utilized are described in Examples 17 and 18. The results from these follow-on experiments are presented in Table 15. In addition to the CPE assay (described above), two additional assay formats were employed, a virus yield reduction (VY) assay and a neutral red uptake (NR), in addition to the CPE test. The NR dye uptake is used to validate the CPE inhibition seen in the initial seen test and utilizes the same 96-well microplates after the CPE has been read (microscopic evaluation). NR is added to the medium; cells not damaged by virus take up a greater amount of dye, which is read on a computerized microplate autoreader. The full method, as described by McManus “Microtiter assay for interferon: microspectrophotometric quantitation of cytopathic effecf”, Appln Environ. Microbiol., 31, 35-38, (1996), the contents of which are incorporated by reference, was used. The dose needed to reduce virus CPE by 50% (EC₅₀) is determined from this dye uptake. Compounds considered active by CPE inhibition and by NR dye uptake were tested again using a VY reduction assay. The effect on reduction of virus yield was assessed by assaying frozen and thawed eluates from each micro well for virus titer by serial dilution onto monolayers of MDCK cells. A known active drug is run in parallel as a positive control. Since PSMA was found to be a potent inhibitor of multiple strains of influenza in tissue culture experiments, it was also tested against human strains of virus adapted to grow in mice (strains NWS/33 and Victoria/37/75 in Table 15).

TABLE 15
Efficacy of PSMA Against Various Strains of Influenza Virus.

| Virus | Strain | Assay* | EC₅₀ wt % | EC₉₀ wt % | CC₉₀ wt % | TI |
|---|---|---|---|---|---|---|
| H1N1 | New Cal./20/99 | VY | — | 0.000018 | — | >556 |
| H1N1 | New Cal./20/99 | CPE | <0.0000032 | — | <0.01 | >3125 |
| H3N2 | California/7/04 | VY | — | 0.0001 | — | >100 |
| H3N2 | California/7/04 | CPE | 0.00015 | — | <0.01 | >67 |
| H1N1 | — | NR | <0.00007 | — | 0.05 | >700 |
| H1N1 | — | CPE | <0.00004 | — | >0.04 | >1000 |
| H3N2 | — | NR | <0.00004 | — | >0.04 | >1000 |
| H3N2 | — | CPE | <0.00007 | — | >0.04 | >1000 |
| H5N1 | — | NR | <0.00009 | — | 0.053 | >588 |
| H5N1 | — | CPE | <0.00004 | — | >0.04 | >1000 |
| Flu B | — | NR | <0.00004 | — | >0.04 | >1000 |
| Flu B | — | CPE | <0.00004 | — | >0.04 | >1000 |
| H1N1 | NWS/33 | NR | <0.000031 | — | 0.042 | >1355 |
| H1N1 | NWS/33 | VY | | <0.000031 | | >2485 |
| H1N1 | NWS/33 | CPE | <0.000031 | — | 0.077 | >2485 |
| H3N2 | Victoria/3/75 | NR | 0.000056 | | 0.036 | 643 |
| H3N2 | Victoria/3/75 | VY | — | 0.000031 | — | 2485 |
| H3N2 | Victoria/3/75 | CPE | 0.000043 | | 0.077 | 1790 |
| Flu B | Sichuan/379/99 | NR | <0.999931 | — | 0.033 | >1065 |
| Flu B | Sichuan/379/99 | VY | — | 0.000056 | — | 1375 |
| Flu B | Sichuan/379/99 | CPE | <0.000031 | — | 0.077 | >2485 |

*The different assay formats include cytopathic effect (CPE), virus yield reduction (VY) and neutral red (NR) uptake.

### Example 21

In vivo, in a dose fmding toxicity study efficacy evaluation, PSMA was administered intranasally to mice using the dose schedule shown in Table 16. In this study, mice were dosed twice a day with 50 ul of test material for five days, and then observed for a total of 21 days. The results after 14 days are presented in Table 16.

TABLE 16
Maximum Tolerated Dose Following Intranasal Administration In Mice.

| PSMA (mg/ul) | | Administered vol. | Regimen | Survivors* | Weight gain/loss |
|---|---|---|---|---|---|
| 10 | mg/ml | 50 ul | 2x a day for 5 days | 0/3 | — |
| 3 | mg/ml | 50 ul | 2x a day for 5 days | 0/3 | — |
| 1 | mg/ml | 50 ul | 2x a day for 5 days | 0/3 | — |
| 0.3 | mg/ml | 50 ul | 2x a day for 5 days | 3/3 | −1.5 gm at day 7 |
| 0.1 | mg/ml | 50 ul | 2x a day for 5 days | 3/3 | −0.6 gm at day 7 |
| 0.03 | mg/ml | 50 ul | 2x a day for 5 days | 3/3 | +0.2 gm at day 7 |

*Survivors at day 14.

From the data obtained in the dose ranging study above, three concentrations of PSMA were found to be acceptable for dosing in an in vivo efficacy analysis, that is, 50 ul twice a day of PSMA at 0.3, 0.1 and 0.03 mg/ml.

As used herein, unless indicated to the contrary, % refers to percentage by weight. Unless indicated to the contrary, the singular refers to the plural and vice versa.

The above embodiments and examples are given to illustrate the scope and spirit of the present invention. These embodiments and examples will make apparent, to those skilled in the art, other embodiments and examples. These other embodiments and examples are within the contemplation of the present invention. Therefore the present invention should be limited only by the appended claims.

We claim:

1. A method for the treatment or prevention of a viral, bacterial, or fungal infection in a host, which comprises administering to the host a therapeutically or prophylactically effective amount of an anionic cellulose-based polymer, a prodrug thereof, or a pharmaceutically acceptable salt of said anionic cellulose based polymer or prodrug, wherein said anionic cellulose based polymer is molecularly dispersed and mostly dissociated in an aqueous solution at pH ranging from about 3 to about 5.

2. A method for the treatment or prevention of a viral, bacterial, or fungal infection in a host, according to claim 1 which comprises administering to the host an effective amount of an anionic cellulose-based polymer, a prodrug thereof, or a pharmaceutically acceptable salt of said anionic cellulose-based polymer or prodrug, wherein said anionic cellulose based polymer comprising a monomer of the following formula 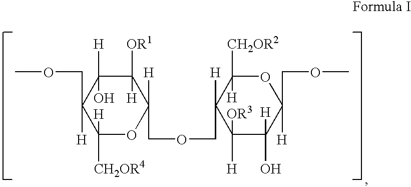
or pharmaceutically acceptable salts thereof;
wherein R¹, R², R³, and R⁴ are the same or different, and are hydrogen, C₁-C₆ hydroxyalkyl, an aliphatic group, an alicyclic group, an aryl group, an arylaliphatic, or an heteroring group or 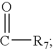
wherein each of said aliphatic group, alicyclic group, aryl group, and heteroring group is independently unsubstituted or substituted by one or more substituents selected from the group consisting of carboxylic acid, sulfuric acid, sulfonic acid, carboxylate, sulfate, sulfonate, and acidic anhydride; R⁷ is hydrogen, C₁-C₆ hydroxyalkyl, an aliphatic group, alicyclic group, an aryl group, arylaliphatic or an heteroring group, wherein which aliphatic groups, alicyclic groups, aryl group and heteroring are independently unsubstituted or substituted by one or more substituents selected from carboxylic acid, sulfuric acid, sulfonic acid, carboxylate, sulfate, sulfonate and acidic anhydride, and, at least one of R¹, R², R³ and R⁴ contains at least one COOH group, wherein the pKa of one of the COOH groups present, or if its salt is present the pKa of the corresponding acid, is less than about 5.0.

3. The method according to claim 2, wherein said aliphatic group, alicyclic group, aryl group, or heteroring group in Formula I is further substituted with one or more hydroxyl groups.

4. The method according to claim 2, wherein said acidic anhydride in Formula I derives from the same or different acids chosen from the group consisting of acetic acid, sulfobenzoic acid, phthalic, trimellitic acid, and other carboxylic acids.

5. The method according to claim 2, wherein at least one of R¹, R², R³, and R⁴ in Formula I is chosen from the group consisting of trimellitic acid, trimesic acid, hemimellitic acid, maleic acid, succinic acid, diethylmalonic acid, trans-aconitic acid, 1,8-naphthalic anhydride, 1,4,5,8-naphthalene tetracarboxylic acid dianhydride, 2-sulfobenzoic acid cyclic anhydride, 4-sulfo-1,8-naphthalic anhydride, tartaric acid, D-mallic acid, L-mallic acid, and vinyl acetic acid.

6. The method according to claim 2 wherein the repeating unit is repeated n times, wherein n is an integer greater than or equal to 3.

7. A method for the treatment or prevention of a viral, bacterial, or fungal infection in a host, which comprises administering to the host a therapeutically effective amount of an anionic acrylic-based polymer, a prodrug thereof, or a pharmaceutically acceptable salt of said anionic acrylic based polymer or prodrug.

8. The method according to claim 7, wherein said anionic acrylic-based polymer is molecularly dispersed and mostly dissociated in an aqueous solution at pH ranging from about 3 to about 5.

9. The method according to claim 7, wherein said anionic acrylic-based polymer comprises a monomer of the following Formula 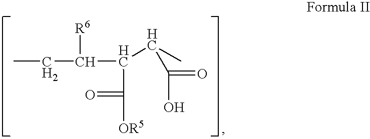
or pharmaceutically acceptable salts thereof;
wherein R⁵ is hydrogen, an aliphatic group, an alicyclic group, an aryl group, aryl aliphatic or an heteroring group; wherein each of said aliphatic group , alicyclic group, aryl group, or heteroring group is independently unsubstituted or substituted by an aliphatic group, alicyclic group, an aryl or aryl aliphatic or R⁵ is 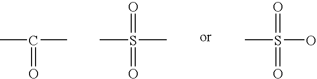
wherein the 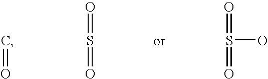
groups are bonded to an aliphatic group, aryl group, alicyclic group, arylaliphatic group or heteroring, which may be unsubstituted or substituted by one or more carbobylic acid moiety, sulfonic acid moiety, sulfur acid moiety and optionally with hydroxy or halide; and each R⁶ is hydrogen, C₁-C₆ alkyl or C₁-C₆ hydroxyalkyl, aryl or SR⁸ or OR⁸, wherein each R⁸ is hydrogen, aliphatic group, alicyclic group, aryl group, or arylaliphatic or heteroring which R⁶ may be unsubstituted or substituted with an aliphatic group, alicyclic group or aryl group, or aryl aliphatic group.

10. The method according to claim 9, wherein said aliphatic group, alicyclic group, aryl group, or heteroring group in Formula II is further substituted with one or more hydroxyl groups.

11. The method according to claim 9, wherein said R⁵ in Formula II is chosen from the group consisting of trimellitic acid, trimesic acid, hemimellitic acid, maleic acid, succinic acid, diethylmalonic acid, trans-aconitic acid, 1,8-naphthalic anhydride, 1,4,5,8-naphthalene tetracarboxylic acid dianhydride, 2-sulfobenzoic acid cyclic anhydride, 4-sulfo-1,8-naphthalic anhydride, tartaric acid, D-mallic acid, L-mallic acid, and vinyl acetic acid.

12. The method according to claim 9, wherein said R⁶ in Formula II is methyl.

13. The method according to claim 2 or 9 wherein the repeating unit is repeated n times, wherein n is an integer of 4 or greater.

14. The method according to claim 13 wherein n is an integer of 10 or greater.

15. The method according to claim 1 or claim 7 wherein the viral infection is caused by a virus selected from the group consisting of HIV-1, HIV-2, HPV, HSV1, HSV2, PIV (parainfluenta), RSV (respiratory synctial virus), rhinoviruses, SARS (severe acute respiratory syndrome) causing virus, influenza virus, Small Pox virus, Cow pox virus, Vaccinia virus, hemorrhagic fever causing virus, Arena virus, Bunyavirus, and Flavirus.

16. The method according to claim 1 or claim 7 wherein the bacterial infection is caused by a bacteria selected from the group consisting of Trichomonas vaginalis, Neisseris gonorrhea Haemopholus ducreyl, Chlamydia trachomatis, Gardnerella vaginalis, Mycoplasma hominis, Mycoplasma capricolum, Mobiluncus curtisii, Prevotella corporis, Calymmatobacterium granulomatis, and Treponema pallidum.

17. The method according to claim 1 or claim 7 wherein the fungal infection is caused by Candida albicans.

18. A method for the treatment or prevention of a virus, bacterial, or fungal infection in a host, which comprises administering to the host a therapeutically effective amount of an anionic cellulose-based polymer, a prodrug thereof, or a pharmaceutically acceptable salt of said anionic cellulose based polymer or prodrug in combination with one or more anti-infective agents.

19. The method according to claim 18 wherein said one or more anti-infective agents are an anti-viral agent, an anti-bacterial agent, an anti-fungal agent, or the combination thereof.

20. The method according to claim 18 wherein the anionic cellulose-based polymer, and the one or more anti-infective agents are administered simultaneously or sequentially.

21. The method according to claim 18 wherein the one or more anti-infective agents are chosen from the group consisting of antiviral protease enzyme inhibitors (PI), virus DNA or RNA or reverse transcriptase (RT) polymerase inhibitors, virus/cell fusion inhibitors, virus integrase enzyme inhibitors, virus/cell binding inhibitors, and/or virus or cell helicase enzyme inhibitors, bacterial cell wall biosynthesis inhibitors, virus or bacterial attachment inhibitors, HIV-1 RT inhibitors, HIV-1 protease inhibitors, HIV-1 fusion inhibitors, polybiguanides (PBGs), herpes virus DNA polymerase inhibitors, herpes virus protease inhibitors, herpes virus fusion inhibitors, herpes virus binding inhibitors, and ribonucleotide reductase inhibitors.

22. A method for the treatment or prevention of a virus, bacterial, or fungal infection in a host, which comprises administering to the host a therapeutically effective amount of an anionic acrylic-based polymer, a prodrug thereof, or a pharmaceutically acceptable salt of said anionic acrylic based polymer or prodrug in combination with one or more anti-infective agents.

23. The method according to claim 22 wherein the one or more anti-infective agents are an anti-viral agent, an anti-bacterial agent, an anti-fungal agent, or the combination thereof.

24. The method according to claim 22 wherein the anionic acrylic-based polymer and the one or more anti-infective agents are administered simultaneously or sequentially.

25. The method according to claim 22 wherein the one or more anti-infective agents are chosen from the group consisting of antiviral protease enzyme inhibitors (PI), virus DNA or RNA or reverse transcriptase (RT) polymerase inhibitors, virus/cell fusion inhibitors, virus integrase enzyme inhibitors, virus/cell binding inhibitors, virus or cell helicase enzyme inhibitors, bacterial cell wall biosynthesis inhibitors, virus or bacterial attachment inhibitors, HIV-1 RT inhibitors, HIV-1 protease inhibitors, HIV-1 fusion inhibitors, polybiguanides (PBGs), herpes virus DNA polymerase inhibitors, herpes virus protease inhibitors, herpes virus fusion inhibitors, herpes virus binding inhibitors, and ribonucleotide reductase inhibitors.

26. A pharmaceutical composition comprising a therapeutically effective amount of the combination of an anionic cellulose-based polymer, a prodrug of said anionic cellulose-based polymer, or a pharmaceutically acceptable salt of said anionic cellulose-based polymer or prodrug and one or more anti-infective agents; and a pharmaceutically acceptable carrier therefor.

27. The pharmaceutical combination composition according to claim 26 wherein the one or more anti-infective agents are chosen from the group consisting of antiviral protease enzyme inhibitors (PI), virus DNA or RNA or reverse transcriptase (RT) polymerase inhibitors, virus/cell fusion inhibitors, virus integrase enzyme inhibitors, virus/cell binding inhibitors, virus or cell helicase enzyme inhibitors, bacterial cell wall biosynthesis inhibitors, virus or bacterial attachment inhibitors, HIV-1 RT inhibitors, HIV-1 protease inhibitors, HIV-1 fusion inhibitors, polybiguanides (PBGs), herpes virus DNA polymerase inhibitors, herpes virus protease inhibitors, herpes virus fusion inhibitors, herpes virus binding inhibitors, and ribonucleotide reductase inhibitors.

28. A pharmaceutical composition comprising a therapeutically effective amount of the combination of anionic acrylic-based polymer, a prodrug of said anionic acrylic-based polymer, or a pharmaceutically acceptable salt of said anionic cellulose based polymer or prodrug and one or more anti-infective agents; and a pharmaceutically acceptable carrier therefor.

29. The pharmaceutical combination composition according to claim 28 wherein the one or more anti-infective agents are chosen from the group consisting of antiviral protease enzyme inhibitors (PI), virus DNA or RNA or reverse transcriptase (RT) polymerase inhibitors, virus/cell fusion inhibitors, virus integrase enzyme inhibitors, virus/cell binding inhibitors, and/or virus or cell helicase enzyme inhibitors, bacterial cell wall biosynthesis inhibitors, virus or bacterial attachment inhibitors, HIV-1 RT inhibitors, HIV-1 protease inhibitors, HIV-1 fusion inhibitors, polybiguanides (PBGs), herpes virus DNA polymerase inhibitors, herpes virus protease inhibitors, herpes virus fusion inhibitors, herpes virus binding inhibitors, and ribonucleotide reductase inhibitors.

30. The method according to any one of claims 21, 25, 27 or claim 29 wherein said HIV-1 RT inhibitors are selected from the group consisting of tenofovir, epivir, zidovudine, and stavudine.

31. The method according to any one of claims 21, 25, 27, or claim 29 wherein said HIV-1 protease inhibitors are selected from the group consisting of saquinavir, ritonavir, nelfmavir, indinavir, amprenavir, lopinavir, atazanavir, tipranavir, and fosamprenavir.

32. The method according to any one of claims 21, 25, 27, or claim 29 wherein said herpes virus DNA polymerase inhibitors are selected from the group consisting of acyclovir, ganciclovir, and cidofovir.

33. A kit comprising:
(a) an anionic cellulose-based polymer, a prodrug of said anionic cellulose-based polymer, or a pharmaceutically acceptable salt of said anionic cellulose-based polymer or prodrug;
(b) one or more anti-infective agents;
(c) a pharmaceutically acceptable carrier, vehicle or diluent; and
(d) a container for containing said compounds described in (a) and (b); wherein said polymer and anti-infective agent are present in amounts effective to result in a therapeutic effect.

34. The kit according to claim 33 wherein the one or more anti-infective agents are an anti-viral agent, an anti-bacterial agent, an anti-fungal agent, or the combination thereof.

35. A kit comprising:
(a) an acrylic-based polymer, a prodrug of said anionic acrylic-based polymer, or a pharmaceutically acceptable salt of said anionic acrylic-based polymer or prodrug;
(b) one or more anti-infective agents;
(c) a pharmaceutically acceptable carrier, vehicle or diluent; and
(d) a container for containing said polymer and anti-infective agent described in (a) and (b), wherein said polymer and said anti-infective agent are present in amounts effective for a therapeutic effect.

36. The kit according to claim 35 wherein the one or more anti-infective agents is an anti-viral agent, an anti-bacterial agent, an anti-fungal agent, or the combination thereof.

37. A vehicle or an adjuvant of a therapeutic or cosmetic composition comprising a polymer having a repeating unit of the following formula: 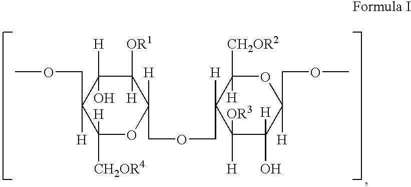
or pharmaceutically acceptable salts thereof;
wherein R¹, R², R³, and R⁴ are the same or different, and are hydrogen, C₁-C₆ hydroxyalkyl, an aliphatic group, preferably C₁-C₆ alkyl, an alicyclic group, an aryl group, an arylaliphatic, or an heteroring group or 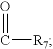
wherein each of said aliphatic group, alicyclic group, aryl group, and heteroring group is independently unsubstituted or substituted by one or more substituents selected from the group consisting of carboxylic acid, sulfuric acid, sulfonic acid, carboxylate, sulfate, sulfonate, and acidic anhydride; R⁷ is hydrogen, C₁-C₆ hydroxyalkyl, an aliphatic group, alicyclic group, an aryl group, arylaliphatic or an heteroring group, wherein the aliphatic groups, alicyclic groups, aryl group and heteroring are independently unsubstituted or substituted by one or more substituents selected from carboxylic acid, sulfuric acid, sulfonic acid, carboxylate, sulfate, sulfonate and acidic anhydride, and, at least one of R¹, R², R³ and R⁴ contains at least one COOH group, wherein the pKa of one of the COOH groups present or if its salt is present, the pKa of the corresponding acid, is less than about 5.0.

38. A thickener for topical administration of a therapeutic or cosmetic composition comprising a polymer having a repeating unit of the following 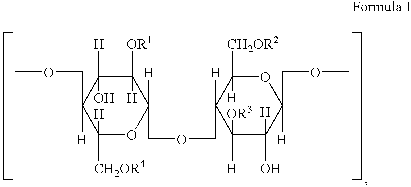
or pharmaceutically acceptable salts thereof;
wherein R¹, R², R³, and R⁴ are the same or different, and are hydrogen, C₁-C₆ hydroxyalkyl, an aliphatic group, preferably C₁-C₆ alkyl, an alicyclic group, an aryl group, an arylaliphatic, or an heteroring group or 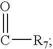
wherein each of said aliphatic group, alicyclic group, aryl group, and heteroring group is independently unsubstituted or substituted by one or more substituents selected from the group consisting of carboxylic acid, sulfuric acid, sulfonic acid, carboxylate, sulfate, sulfonate, and acidic anhydride; R⁷ is hydrogen, C₁-C₆ hydroxyalkyl, an aliphatic group, preferably C₁-C₆ alkyl, alicyclic group, an aryl group, arylaliphatic or an heteroring group, wherein the aliphatic groups, alicyclic groups, aryl group and heteroring are independently unsubstituted or substituted by one or more substituents selected from carboxylic acid, sulfuric acid, sulfonic acid, carboxylate, sulfate, sulfonate and acidic anhydride, and, at least one of R¹, R², R³ and R⁴ contains at least one COOH group, wherein the pKa of one of the COOH groups present or if its salt is presents the pKa of the corresponding acid is less than about 5.0.

39. A vehicle or an adjuvant of a therapeutic or cosmetic composition comprising a polymer having a repeating unit of the following formula: 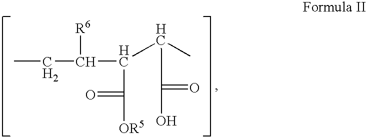
or pharmaceutically acceptable salts thereof;
wherein R⁵ is hydrogen, an aliphatic group , an alicyclic group, an aryl group, aryl aliphatic or an heteroring group; wherein each of said aliphatic group , alicyclic group, aryl group, or heteroring group is independently unsubstituted or substituted by an aliphatic group, alicyclic group, an aryl or aryl aliphatic or R⁵ is 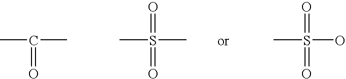
wherein the 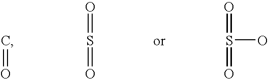
groups are bonded to an aliphatic group, aryl group, alicyclic group, arylaliphatic group or heteroring, which groups may be unsubstituted or substituted by one or more carbobylic acid moiety, sulfonic acid moiety, sulfuric acid moiety and optionally hydroxy or halide; and each R⁶ is hydrogen, C₁-C₆ alkyl or C₁-C₆ hydroxyalkyl, aryl or SR⁸ or OR⁸, wherein each R⁸ is hydrogen, aliphatic group, alicyclic group, aryl group, or arylaliphatic or heteroring which R⁶ may be unsubstituted or substituted with an aliphatic group, alicyclic group or aryl group, or aryl aliphatic group.

40. A thickener for topical administration of a therapeutic or cosmetic composition comprising a polymer having a repeating unit of the following formula: 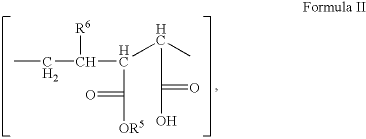
or pharmaceutically acceptable salts thereof;
wherein R⁵ is hydrogen, an aliphatic group, an alicyclic group, an aryl group, aryl aliphatic or an heteroring group; wherein each of said aliphatic group, alicyclic group, aryl group, or heteroring group is independently unsubstituted or substituted by an aliphatic group, alicyclic group, an aryl or aryl aliphatic or aliphatic aryl group or R⁵ is 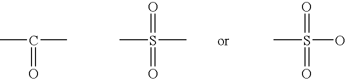
wherein the 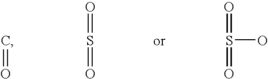
groups are bonded to an aliphatic group, aryl group, alicyclic group, arylaliphatic groups or heteroring, which groups may be unsubstituted or substituted by one or more carbobylic acid moiety, sulfur acid moiety, sulfonic acid moiety and optionally with hydroxy or halide; and each R⁶ is hydrogen, C₁-C₆ alkyl or C₁-C₆ hydroxyalkyl, aryl or SR⁸ or OR⁸, wherein each R⁸ is hydrogen, aliphatic group, alicyclic group, aryl group, arylaliphatic or heteroring which R⁶ may be unsubstituted or substituted with an aliphatic group, alicyclic group or aryl group, or aryl aliphatic group.

41. The method according to claim 1 or claim 7 wherein the virus is an influenza virus.

42. The method according to claim 41 wherein the polymer is PSMA.

43. A method for the treatment or prevention of a disease caused by or associated with a viral, bacterial or fungal infection in a host, which comprises administering to the host a therapeutically or prophylactically effective amount of an anionic cellulose-based polymer, a prodrug thereof, or a pharmaceutically acceptable salt of said anionic cellulose based polymer or prodrug, wherein said anionic cellulose based polymer is molecularly dispersed and mostly dissociated in an aqueous solution at pH ranging from about 3 to about 5.

44. The method according to claim 43, wherein the anionic cellulose based polymer comprises a repeating unit of the following: 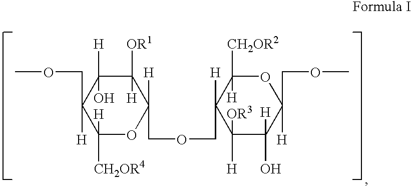
or pharmaceutically acceptable salts thereof;
wherein R¹, R², R³, and R⁴ are the same or different, and are hydrogen, C₁-C₆ hydroxyalkyl, an aliphatic group, an alicyclic group, an aryl group, arylaliphatic, or an heteroring group or 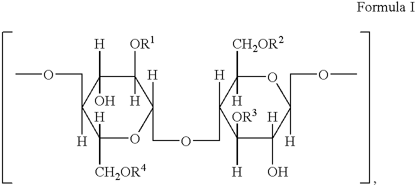
wherein each of said aliphatic group, alicyclic group, aryl group, and heteroring group is independently unsubstituted or substituted by one or more substituents selected from the group consisting of carboxylic acid, sulfuric acid, sulfonic acid, carboxylate, sulfate, sulfonate, and acidic anhydride; R⁷ is hydrogen, C₁-C₆ hydroxyalkyl, an aliphatic group, alicyclic group, an aryl group, arylaliphatic group or an heteroring group, wherein which aliphatic groups, alicyclic groups, aryl group and heteroring are independently unsubstituted or substituted by one or more substituents selected from carboxylic acid, sulfuric acid, sulfonic acid, carboxylate, sulfate, sulfonate and acidic anhydride, and, at least one of R¹, R², R³ and R⁴ contains at least one COOH group, wherein the pKa of one of the COOH groups present or if its salt is presents the pKa of the corresponding acid is less than about 5.0.

45. The method according to claim 44 wherein said aliphatic group, alicyclic group, an aryl group and heteroring group in Formula I is further substituted with one or more hydroxyl groups.

46. The method according to claim 44, wherein said acidic anhydride in Formula I derives from the same or different acids chosen from the group consisting of acetic acid, sulfobenzoic acid, phthalic, trimellitic acid, and other carboxylic acids.

47. The method according to claim 44, wherein at least one of R¹, R², R³, and R⁴ in Formula I is chosen from the group consisting of trimellitic acid, trimesic acid, hemimellitic acid, maleic acid, succinic acid, diethylmalonic acid, trans-aconitic acid, 1 ,8-naphthalic anhydride, 1,4,5,8-naphthalene tetracarboxylic acid dianhydride, 2-sulfobenzoic acid cyclic anhydride, 4-sulfo-1,8-naphthalic anhydride, tartaric acid, D-mallic acid, L-mallic acid, and vinyl acetic acid.

48. The method according to claim 44 wherein the repeating unit is repeated n times, wherein n is an integer greater than or equal to 3.

49. A method for the treatment or prevention of a disease caused by or associated with viral, bacterial, or fungal infection in a host, which comprises administering to the host an effective amount of an anionic acrylic-based polymer, a prodrug thereof, or a pharmaceutically acceptable salt of said anionic acrylic based polymer or prodrug.

50. The method according to claim 49, wherein said anionic acrylic-based polymer is molecularly dispersed and mostly dissociated in an aqueous solution at pH ranging from about 3 to about 5.

51. The method according to claim 50, wherein said anionic acrylic-based polymer comprises a repeating unit of the following Formula 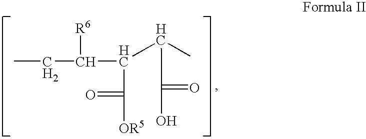
or pharmaceutically acceptable salts thereof;
wherein R⁵ is hydrogen, an aliphatic group, an alicyclic group, an aryl group, aryl aliphatic or an heteroring group; wherein each of said aliphatic group, alicyclic group, aryl group, or heteroring group is independently unsubstituted or substituted by an aliphatic group, alicyclic group, an aryl or aryl aliphatic or R⁵ is 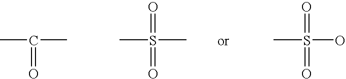
wherein the 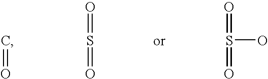
groups are independently bonded to an aliphatic group, aryl group, alicyclic group or heteroring, which may be unsubstituted or substituted by one or more carbobylic acid moiety, sulfonic acid moiety, sulfur acid moiety and optionally hydroxy or halide; and each R⁶ is hydrogen, C₁-C₆ alkyl or C₁-C₆ hydroxyalkyl, aryl or SR⁸ or OR⁸, wherein each R⁸ is hydrogen, aliphatic group, alicyclic group, aryl group, or arylaliphatic or heteroring which R⁶ may be unsubstituted or substituted with an aliphatic group, alicyclic group or aryl group, or aryl aliphatic group.

52. The method according to claim 51, wherein said aliphatic group, alicyclic group, aryl group, or heteroring group in Formula II is further substituted with one or more hydroxyl groups.

53. The method according to claim 51, wherein said R⁵ in Formula II is chosen from the group consisting of trimellitic acid, trimesic acid, hemimellitic acid, maleic acid, succinic acid, diethylmalonic acid, trans-aconitic acid, 1,8-naphthalic anhydride, 1,4,5,8-naphthalene tetracarboxylic acid dianhydride, 2-sulfobenzoic acid cyclic anhydride, 4-sulfo-1,8-naphthalic anhydride, tartaric acid, D-mallic acid, L-mallic acid, and vinyl acetic acid.

54. The method according to claim 51, wherein said R⁶ in Formula II is methyl.

55. The method according to claim 44 or 51 wherein the repeating unit is repeated in times, wherein n is an integer of 4 or greater.

56. The method according to claim 55 wherein n is an integer of 10 or greater.

57. The method according to claim 43 or claim 49 wherein the viral infection is caused by a virus selected from the group consisting of HIV-1, HIV-2, HPV, HSV1, HSV2, PIV (parinfluenta), RSV (respiratory synctial virus), SARS (severe acute respiratory syndrome) causing virus, influenza virus, Small pox virus, Cow pox virus, Vaccinia virus, hemorrhagic fever causing virus, Arena virus, Bunyavirus and Flavirus.

58. The method according to claim 43 or claim 49 wherein the bacterial infection is caused by bacteria selected from the group consisting of Trichomonas vaginalis, Neisseris gonorrhea Haemopholus ducreyl, Chlamydia trachomatis, Gardnerella vaginalis, Mycoplasma hominis, Mycoplasma capricolum, Mobiluncus curtisii, Prevotella corporis, Calymmatobacterium granulomatis, and Treponema pallidum.

59. The method according to claim 43 or claim 49 wherein the fungal infection is caused by Candida albicans.
